(19) United States
(12) Patent Application Publication
(10) Pub. No.: US 2024/0352434 A1
(43) Pub. Date: Oct. 24, 2024

(54) COMPOSITIONS COMPRISING A VARIANT CAS12I3 POLYPEPTIDE AND USES THEREOF

(71) Applicant: Arbor Biotechnologies, Inc., Cambridge, MA (US)

(72) Inventors: Derek Michael Cerchione, Cambridge, MA (US); Shaorong Chong, Arlington, MA (US); Brendan Jay Hilbert, Natick, MA (US)

(21) Appl. No.: 18/682,774

(22) PCT Filed: Aug. 12, 2022
(86) PCT No.: PCT/US2022/074906

§ 371 Date: Feb. 9, 2024

Related U.S. Application Data
(60) Provisional application No. 63/232,432, filed on Aug. 12, 2021.

(51) Int. Cl. C12N 9/22; A61K 38/46; C12N 15/11; C12N 15/90

(52) U.S. Cl. CPC C12N 9/22; A61K 38/465; C12N 15/11; C12N 15/907; C12N 2310/20

(57) ABSTRACT

The present invention relates to variant Cas12i3 polypeptides, methods of preparing the variant Cas12i3 polypeptides, processes for characterizing the variant Cas12i3 polypeptides, compositions, gene editing systems, and cells comprising the variant Cas12i3 polypeptides, and methods of using the variant Cas12i3 polypeptides. The invention further relates to complexes comprising the variant Cas12i3 polypeptides, methods of producing the complexes, processes for characterizing the complexes, cells comprising the complexes, and methods of using the complexes.

### SEQUENCE LISTING

The instant application contains a Sequence Listing which has been submitted electronically in XML format and is hereby incorporated by reference in its entirety. Said XML copy, created on Jul. 27, 2022, is named 51451-036WO2_Sequence_Listing_7_27_22 and is 21,138 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art.

Although this invention disclosed herein is not limited to specific advantages or functionalities, the invention provides a variant Cas12i3 polypeptide, wherein the variant Cas12i3 polypeptide comprises an amino acid sequence at least 95% identical to SEQ ID NO: 3 and comprises one or more mutations relative to a wild-type Cas12i3 polypeptide of SEQ ID NO: 3.

In some aspects, the one or more mutations in the variant Cas12i3 polypeptide are at one or more of positions S536, S559, R565, H566, S571, K583, K586, L592, M595, E596, 1630, T631, N646, L703, S704, N711, C724, S738, S801, T844, T977, and/or T1040 of SEQ ID NO: 3.

In some aspects, the one or more mutations are amino acid substitutions, which optionally are one or more of S536R, S559R, R565G, H566R, S571R, K583R, K586R, L592R, M595R, E596R, I630R, T631R, N646R, L703R, S704R, N711R, C724R, S738R, S801R, T844R, T977R, T1040R, or a combination thereof.

In some aspects, the variant Cas12i3 polypeptide comprises: (a) mutations at positions S536, N711, and S801, which optionally are amino acid substitutions of S536R, N711R, and S801R; (b) mutations at positions S536, N711, C724, and S801, which optionally are amino acid substitutions of S536R, N711R, C724R, and S801R; (c) mutations at positions S536, N711, and C724, which optionally are amino acid substitutions of S536R, N711R, and C724R; (d) mutations at positions S536 and N711, which optionally are amino acid substitutions of S536R and N711R; (c) mutations at positions S536, K583, N711, and S801, which optionally are amino acid substitutions of S536R, K583R, N711R, and S801R; (f) mutations at positions S536, K583, N711, and C724, which optionally are amino acid substitutions of S536R, K583R, N711R, and C724R; (g) mutations at positions S536, K583, C724, and S801, which optionally are amino acid substitutions of S536R, K583R, C724R, and S801R; (h) mutations at positions S536, K583, and C724, which optionally are amino acid substitutions of S536R, K583R, and C724R; (i) mutations at positions S536 and K583, which optionally are amino acid substitutions of S536R and K583R; (j) mutations at positions S536, C724, and S801, which optionally are amino acid substitutions of S536R, C724R, and S801R; (k) mutations at positions S536 and C724, which optionally are amino acid substitutions of S536R and C724R; (l) mutations at positions N711 and S801, which optionally are amino acid substitutions of N711R and S801R; (m) mutations at positions N711, C724, and S801, which optionally are amino acid substitutions of N711R, C724R, and S801R; (n) mutations at positions N711 and C724, which optionally are amino acid substitutions of N711R and C724R; (o) mutations at positions K583 and S801, which optionally are amino acid substitutions of K583R and S801R; (p) mutations at positions K583, N711, and S801, which optionally are amino acid substitutions of K583R, N711R, and S801R; (q) mutations at positions K583, N711, C724, and S801, which optionally are amino acid substitutions of K583R, N711R, C724R, and S801R; (r) mutations at positions K583, N711, and C724, which optionally are amino acid substitutions of K583R, N711R, and C724R; (s) mutations at positions K583 and N711, which optionally are amino acid substitutions of K583R and N711R; (t) mutations at positions K583, C724, and S801, which optionally are amino acid substitutions of K583R, C724R, and S801R; (u) mutations at positions K583 and C724, which optionally are amino acid substitutions of K583R and C724R; or (v) mutations at positions C724 and S801, which optionally are amino acid substitutions of C724R and S801R.

In some aspects, the variant Cas12i3 polypeptide comprises an amino acid sequence at least 95% identical to SEQ ID NO: 7 and comprises mutations at positions K583, N711, and C724.

In some aspects, the mutations are amino acid substitutions of K583R, N711R, and C724R.

In some aspects, the variant Cas12i3 polypeptide further comprises one or more substitutions of Table 2.

In some aspects, the variant Cas12i3 polypeptide comprises the amino acid sequence of SEQ ID NO: 7.

In some aspects, the variant Cas12i3 polypeptide comprises a RuvC domain or a split RuvC domain.

In some aspects, the variant Cas12i3 polypeptide comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

The invention further provides a first nucleic acid encoding a variant Cas12i3 polypeptide described herein.

The invention further provides a gene editing system comprising the variant Cas12i3 polypeptide described herein.

The invention further provides a gene editing system comprising a first nucleic acid described herein.

In some aspects, the first nucleic acid is a messenger RNA (mRNA).

In some aspects, the first nucleic acid is codon-optimized for expression in a cell.

In some aspects, the first nucleic acid is included in a viral vector, which optionally is an adeno-associated viral (AAV) vector.

In some aspects, the gene editing system further comprises an RNA guide or a second nucleic acid encoding the RNA guide.

In some aspects, the RNA guide comprises a direct repeat sequence and a spacer sequence. In some aspects, the direct repeat comprises: (a) nucleotide 1 through nucleotide 36 of a sequence that is at least 90% identical to SEQ ID NO: 4 or SEQ ID NO: 5; (b) nucleotide 2 through nucleotide 36 of a sequence that is at least 90% identical to SEQ ID NO: 4 or SEQ ID NO: 5; (c) nucleotide 3 through nucleotide 36 of a sequence that is at least 90% identical to SEQ ID NO: 4 or SEQ ID NO: 5; (d) nucleotide 4 through nucleotide 36 of a sequence that is at least 90% identical to SEQ ID NO: 4 or SEQ ID NO: 5; (c) nucleotide 5 through nucleotide 36 of a sequence that is at least 90% identical to SEQ ID NO: 4 or SEQ ID NO: 5; (f) nucleotide 6 through nucleotide 36 of a sequence that is at least 90% identical to SEQ ID NO: 4 or SEQ ID NO: 5; (g) nucleotide 7 through nucleotide 36 of a sequence that is at least 90% identical to SEQ ID NO: 4 or SEQ ID NO: 5; (h) nucleotide 8 through nucleotide 36 of a sequence that is at least 90% identical to SEQ ID NO: 4 or SEQ ID NO: 5; (i) nucleotide 9 through nucleotide 36 of a sequence that is at least 90% identical to SEQ ID NO: 4 or SEQ ID NO: 5; (j) nucleotide 10 through nucleotide 36 of a sequence that is at least 90% identical to SEQ ID NO: 4 or SEQ ID NO: 5; (k) nucleotide 11 through nucleotide 36 of a sequence that is at least 90% identical to SEQ ID NO: 4 or SEQ ID NO: 5; (l) nucleotide 12 through nucleotide 36 of a sequence that is at least 90% identical to SEQ ID NO: 4 or SEQ ID NO: 5; (m) nucleotide 13 through nucleotide 36 of a sequence that is at least 90% identical to SEQ ID NO: 4 or SEQ ID NO: 5; (n) nucleotide 14 through nucleotide 36 of a sequence that is at least 90% identical to SEQ ID NO: 4 or SEQ ID NO: 5; or (o) a sequence that is at least 90% identical to a sequence of SEQ ID NO: 6 or a portion thereof.

In some aspects, the direct repeat comprises: (a) nucleotide 1 through nucleotide 36 of a sequence that is at least 95% identical to SEQ ID NO: 4 or SEQ ID NO: 5; (b) nucleotide 2 through nucleotide 36 of a sequence that is at least 95% identical to SEQ ID NO: 4 or SEQ ID NO: 5; (c) nucleotide 3 through nucleotide 36 of a sequence that is at least 95% identical to SEQ ID NO: 4 or SEQ ID NO: 5; (d) nucleotide 4 through nucleotide 36 of a sequence that is at least 95% identical to SEQ ID NO: 4 or SEQ ID NO: 5; (e) nucleotide 5 through nucleotide 36 of a sequence that is at least 95% identical to SEQ ID NO: 4 or SEQ ID NO: 5; (f) nucleotide 6 through nucleotide 36 of a sequence that is at least 95% identical to SEQ ID NO: 4 or SEQ ID NO: 5; (g) nucleotide 7 through nucleotide 36 of a sequence that is at least 95% identical to SEQ ID NO: 4 or SEQ ID NO: 5; (h) nucleotide 8 through nucleotide 36 of a sequence that is at least 95% identical to SEQ ID NO: 4 or SEQ ID NO: 5; (i) nucleotide 9 through nucleotide 36 of a sequence that is at least 95% identical to SEQ ID NO: 4 or SEQ ID NO: 5; (j) nucleotide 10 through nucleotide 36 of a sequence that is at least 95% identical to SEQ ID NO: 4 or SEQ ID NO: 5; (k) nucleotide 11 through nucleotide 36 of a sequence that is at least 95% identical to SEQ ID NO: 4 or SEQ ID NO: 5; (l) nucleotide 12 through nucleotide 36 of a sequence that is at least 95% identical to SEQ ID NO: 4 or SEQ ID NO: 5; (m) nucleotide 13 through nucleotide 36 of a sequence that is at least 95% identical to SEQ ID NO: 4 or SEQ ID NO: 5; (n) nucleotide 14 through nucleotide 36 of a sequence that is at least 95% identical to SEQ ID NO: 4 or SEQ ID NO: 5; or (o) a sequence that is at least 95% identical to a sequence of SEQ ID NO: 6 or a portion thereof.

In some aspects, the direct repeat comprises: (a) nucleotide 1 through nucleotide 36 of SEQ ID NO: 4 or SEQ ID NO: 5; (b) nucleotide 2 through nucleotide 36 of SEQ ID NO: 4 or SEQ ID NO: 5; (c) nucleotide 3 through nucleotide 36 of SEQ ID NO: 4 or SEQ ID NO: 5; (d) nucleotide 4 through nucleotide 36 of SEQ ID NO: 4 or SEQ ID NO: 5; (e) nucleotide 5 through nucleotide 36 of SEQ ID NO: 4 or SEQ ID NO: 5; (f) nucleotide 6 through nucleotide 36 of SEQ ID NO: 4 or SEQ ID NO: 5; (g) nucleotide 7 through nucleotide 36 of SEQ ID NO: 4 or SEQ ID NO: 5; (h) nucleotide 8 through nucleotide 36 of SEQ ID NO: 4 or SEQ ID NO: 5; (i) nucleotide 9 through nucleotide 36 of SEQ ID NO: 4 or SEQ ID NO: 5; (j) nucleotide 10 through nucleotide 36 of SEQ ID NO: 4 or SEQ ID NO: 5; (k) nucleotide 11 through nucleotide 36 of SEQ ID NO: 4 or SEQ ID NO: 5; (1) nucleotide 12 through nucleotide 36 of SEQ ID NO: 4 or SEQ ID NO: 5; (m) nucleotide 13 through nucleotide 36 of SEQ ID NO: 4 or SEQ ID NO: 5; (n) nucleotide 14 through nucleotide 36 of SEQ ID NO: 4 or SEQ ID NO: 5; or (0) SEQ ID NO: 6 or a portion thereof.

In some aspects, the spacer sequence comprises between 15 and 35 nucleotides in length.

In some aspects, the system comprises the second nucleic acid encoding the RNA guide.

In some aspects, the second nucleic acid encoding the RNA guide is located in a viral vector.

In some aspects, the viral vector comprises the both the first nucleic acid encoding the Cas12i3 polypeptide and the second nucleic acid encoding the RNA guide.

In some aspects, the system comprises the first nucleic acid encoding the Cas12i3 polypeptide, which is located in a first viral vector, and the system comprises the second nucleic acid encoding the RNA guide, which is located in a second viral vector.

In some aspects, the gene editing system is present in a delivery composition comprising a nanoparticle, a liposome, an exosome, a microvesicle, or a gene-gun.

In some aspects, the spacer sequence is specific to a target sequence within a target nucleic acid.

In some aspects, the target sequence is adjacent to a protospacer adjacent motif (PAM) comprising the motif of 5′-TTN-3′ or 5′-NTTN-3′.

In some aspects, the variant Cas12i3 polypeptide exhibits enhanced enzymatic activity (e.g., nuclease activity), enhanced binding activity (e.g., binding activity to an RNA guide), enhanced binding specificity (e.g., binding specificity to an RNA guide), and/or enhanced stability relative to the wild-type Cas12i3 polypeptide of SEQ ID NO: 3.

In some aspects, the variant Cas12i3 polypeptide and the RNA guide exhibit enhanced binding activity to a target nucleic acid, enhanced binding specificity to the target nucleic acid, and/or enhanced stability relative to the wild-type Cas12i3 polypeptide of SEQ ID NO: 3 and the RNA guide.

The invention further provides a composition comprising a variant Cas12i3 polypeptide or a gene editing system described herein.

In some aspects, the composition is a pharmaceutical composition.

The invention further provides a cell comprising a variant Cas12i3 polypeptide, a gene editing system, or a composition described herein.

In some aspects, the cell is a eukaryotic cell or a prokaryotic cell.

In some aspects, the cell is a mammalian cell or a plant cell.

In some aspects, the cell is a human cell.

The invention further provides a method of producing a variant Cas12i3 polypeptide, a gene editing system, or a composition as described herein.

In some aspects, the method is for producing the variant Cas12i3 polypeptide and the method comprises transforming a host cell with an expression vector expressing a first nucleic acid described herein.

In some aspects, the host cell is ex vivo or in vitro.

In some aspects, the host cell is in vivo.

The invention further provides a method of delivering a variant Cas12i3 polypeptide, a gene editing system, or a composition of described herein.

In some aspects, the Cas12i3 polypeptide, the gene editing system, or the composition is present within a delivery composition, which optionally is selected from the group consisting of a nanoparticle, a liposome, an exosome, a microvesicle, or a gene-gun.

In some aspects, the delivery composition is delivered to a cell using a method selected from the group consisting of transfection, electroporation, nucleofection, viral delivery, microinjection, microprojectile bombardment, fugene, direct sonic loading, cell squeezing, optical transfection, protoplast fusion, impalefection, magnetofection, exosome-mediated transfer, lipid nanoparticle-mediated transfer, and any combination thereof.

In some aspects, the delivery is to a cell, which is a eukaryotic cell or a prokaryotic cell.

In some aspects, the cell is a mammalian cell or a plant cell.

In some aspects, the cell is a human cell.

The invention further provides a method of editing a cell, comprising administering a gene editing system or composition described herein to a cell.

In some aspects, the gene editing system is comprised within a delivery composition, which optionally is selected from the group consisting of a nanoparticle, a liposome, an exosome, a microvesicle, or a gene-gun.

In some aspects, the cell is ex vivo or in vitro.

In some aspects, the cell is in vivo.

In some aspects, the cell is a eukaryotic cell or a prokaryotic cell.

In some aspects, the cell is a mammalian cell or a plant cell.

In some aspects, the cell is a human cell.

### Definitions

The present invention will be described with respect to particular embodiments and with reference to certain Figures, but the invention is not limited thereto but only by the claims. Terms as set forth hereinafter are generally to be understood in their common sense unless indicated otherwise.

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including.” as well as other forms, such as “includes” and “included.” is not limiting.

Generally, nomenclature used in connection with cell and tissue culture, molecular biology, immunology, microbiology, genetics, and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “activity” refers to a biological activity. In some embodiments, effector activity includes enzymatic activity, e.g., catalytic ability of an effector. For example, effector activity can include nuclease activity. In some embodiments, effector activity includes binding activity, e.g., binding activity of an effector to an RNA guide and/or target nucleic acid.

As used herein, the term “complex” refers to a grouping of two or more molecules. In some embodiments, the complex comprises a polypeptide and a nucleic acid molecule interacting with (e.g. binding to, coming into contact with, adhering to) one another.

As used herein, the term “binary complex” refers to a grouping of two molecules (e.g., a polypeptide and a nucleic acid molecule). In some embodiments, a binary complex refers to a grouping of a polypeptide and a targeting moiety (e.g., an RNA guide). In some embodiments, a binary complex refers to a ribonucleoprotein (RNP). As used herein, the term “variant binary complex” refers to the grouping of a variant Cas12i3 polypeptide and RNA guide. As used herein, the term “parent binary complex” refers to the grouping of a parent polypeptide and RNA guide or a reference polypeptide and RNA guide.

As used herein, the term “ternary complex” refers to a grouping of three molecules (e.g., a polypeptide and two nucleic acid molecules). In some embodiments, a “ternary complex” refers to a grouping of a polypeptide, an RNA molecule, and a DNA molecule. In some embodiments, a ternary complex refers to a grouping of a polypeptide, a targeting moiety (e.g., an RNA guide), and a target nucleic acid (e.g., a target DNA molecule). In some embodiments, a “ternary complex” refers to a grouping of a binary complex (e.g., a ribonucleoprotein) and a third molecule (e.g., a target nucleic acid).

As used herein, the term “domain” refers to a distinct functional and/or structural unit of a polypeptide. In some embodiments, a domain may comprise a conserved amino acid sequence.

As used herein, the term “insertion” refers to a gain of amino acids in a polypeptide sequence. In some embodiments, the term “insertion” refers to a gain of amino acids relative to the number of amino acids of a Cas12i3 polypeptide (e.g., a polypeptide of SEQ ID NO: 3) or of a variant Cas12i3 polypeptide (e.g., a polypeptide of SEQ ID NO: 7).

As used herein, the terms “parent.” “parent polypeptide.” and “parent sequence” refer to an original polypeptide (e.g., reference or starting polypeptide) to which an alteration is made to produce a variant Cas12i3 polypeptide of the present invention.

As used herein, the term “protospacer adjacent motif” or “PAM” refers to a DNA sequence adjacent to a target sequence. In a double-stranded DNA molecule, the strand containing the PAM motif is called the “PAM-strand” and the complementary strand is called the “non-PAM strand.” The RNA guide binds to a site in the non-PAM strand that is complementary to a target sequence.

In some embodiments, the PAM strand is a coding (e.g., sense) strand. In other embodiments, the PAM strand is a non-coding (e.g., antisense strand). Since an RNA guide binds the non-PAM strand via base-pairing, the non-PAM strand is also known as the target strand, while the PAM strand is also known as the non-target strand.

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, a first sequence is adjacent to a second sequence if the two sequences are separated by up to 2 nucleotides, up to 5 nucleotides, up to 8 nucleotides, up to 10 nucleotides, up to 12 nucleotides, or up to 15 nucleotides. In some embodiments, a first sequence is adjacent to a second sequence if the two sequences are separated by 2-5 nucleotides, 4-6 nucleotides, 4-8 nucleotides, 4-10 nucleotides, 6-8 nucleotides, 6-10 nucleotides, 6-12 nucleotides, 8-10 nucleotides, 8-12 nucleotides, 10-12 nucleotides, 10-15 nucleotides, or 12-15 nucleotides.

As used herein, the terms “reference composition.” “reference molecule.” “reference sequence.” and “reference” refer to a control, such as a negative control or a parent (e.g., a parent sequence, a parent protein, or a wild-type protein). For example, a reference molecule refers to a polypeptide to which a variant Cas12i3 polypeptide is compared. Likewise, a reference RNA guide refers to a targeting moiety to which a modified RNA guide is compared. The variant or modified molecule may be compared to the reference molecule on the basis of sequence (e.g., the variant or modified molecule may have X % sequence identity or homology with the reference molecule), thermostability, or activity (e.g., the variant or modified molecule may have X % of the activity of the reference molecule). For example, a variant or modified molecule may be characterized as having no more than 10% of an activity of the reference polypeptide or may be characterized as having at least 10% greater of an activity of the reference polypeptide. Examples of reference polypeptides include naturally occurring unmodified polypeptides, e.g., naturally occurring polypeptides from archaea or bacterial species. In certain embodiments, the reference polypeptide is a naturally occurring polypeptide having the closest sequence identity or homology with the variant Cas12i3 polypeptide to which it is being compared. In certain embodiments, the reference polypeptide is a parental molecule having a naturally occurring or known sequence on which a mutation has been made to arrive at the variant Cas12i3 polypeptide.

As used herein, the term “RNA guide” or “RNA guide sequence” refers to any RNA molecule or a modified RNA molecule that facilitates the targeting of a polypeptide (e.g., a Cas12i3 polypeptide) described herein to a target sequence. For example, an RNA guide can be a molecule that is designed to be complementary to a specific nucleic acid sequence. An RNA guide may comprise a spacer sequence and a direct repeat (DR) sequence. In some instances, the RNA guide can be a modified RNA molecule comprising one or more deoxyribonucleotides, for example, in a DNA-binding sequence contained in the RNA guide, which binds a sequence complementary to the target sequence. In some examples, the DNA-binding sequence may contain a DNA sequence or a DNA/RNA hybrid sequence. The terms CRISPR RNA (crRNA), pre-crRNA, and mature crRNA are also used herein to refer to an RNA guide.

As used herein, the term “substantially identical” refers to a sequence, polynucleotide, or polypeptide, that has a certain degree of identity to a reference sequence.

As used herein, the term “target sequence” refers to a DNA fragment adjacent to a PAM motif (on the PAM strand). The complementary region of the target sequence is on the non-PAM strand. A target sequence may be immediately adjacent to the PAM motif. Alternatively, the target sequence and the PAM may be separately by a small sequence segment (e.g., up to 5 nucleotides, for example, up to 4, 3, 2, or 1 nucleotide). A target sequence may be located at the 3′ end of the PAM motif or at the 5′ end of the PAM motif, depending upon the CRISPR nuclease that recognizes the PAM motif, which is known in the art. For example, a target sequence is located at the 3′ end of a PAM motif for a Cas12i3 polypeptide (e.g., a Cas12i3 polypeptide such as those disclosed herein).

As used herein, the terms “variant Cas12i3 polypeptide” and “variant effector polypeptide” refer to a polypeptide comprising an alteration, e.g., but not limited to, a substitution, insertion, deletion, addition, and/or fusion, at one or more residue positions, compared to a parent polypeptide. As used herein, the terms “variant Cas12i3 polypeptide” and “variant effector polypeptide” refer to a polypeptide comprising an alteration as compared to the polypeptide of SEQ ID NO: 3.

### BRIEF DESCRIPTION OF THE DRAWINGS

FIG. 1 is a graph showing indels induced in AAVS1, EMX1, and VEGFA targets by wild-type Cas12i3 (SEQ ID NO: 3) and Cas12i3 variant polypeptides having single amino acid substitutions in mammalian cells.

FIG. 2 is a graph showing indels induced in AAVS1, EMX1, and VEGFA targets by wild-type Cas12i3 (SEQ ID NO: 3) and Cas12i3 variant polypeptides having two-five substitutions in mammalian cells.

### DETAILED DESCRIPTION

In some aspects, the present invention provides novel variants of the effector of SEQ ID NO: 3, compositions or gene editing systems comprising the variants, and methods of preparation and use thereof. In other aspects, the present invention further provides complexes comprising a variant of the effector of SEQ ID NO: 3 and compositions or gene editing systems, methods of preparation, and use thereof. In some aspects, a composition or gene editing system comprising a complex having one or more characteristics is described herein. In some aspects, a method of delivering a composition or gene editing system comprising the complex is described.

### Compositions

In some embodiments, a composition or gene editing system of the invention includes a variant Cas12i3 polypeptide that exhibits enhanced enzymatic activity, enhanced binding activity, enhanced binding specificity, and/or enhanced stability relative to a parent polypeptide. In some embodiments, a composition or gene editing system of the invention includes a complex comprising a variant Cas12i3 polypeptide that exhibits enhanced enzymatic activity, enhanced binding activity, enhanced binding specificity, and/or enhanced stability relative to a parent complex.

In some embodiments, a composition or gene editing system of the invention includes a variant Cas12i3 polypeptide and an RNA guide. In some embodiments, a composition or gene editing system of the invention includes a variant binary complex comprising a variant Cas12i3 polypeptide and an RNA guide.

In some aspects of the composition or gene editing system, the variant Cas12i3 polypeptide has increased complex formation (e.g., increased binary complex formation) with the RNA guide as compared to a parent polypeptide. In some aspects of the composition or gene editing system, the variant Cas12i3 polypeptide and the RNA guide have a greater binding affinity, as compared to a parent polypeptide and the RNA guide. In some aspects of the composition or gene editing system, the variant Cas12i3 polypeptide and the RNA guide have stronger protein-RNA interactions (e.g., ionic interactions), as compared to a parent polypeptide and the RNA guide. In some aspects of the composition or gene editing system, the variant binary complex is more stable than a parent binary complex.

In some embodiments, a composition or gene editing system of the invention includes a variant Cas12i3 polypeptide, an RNA guide, and a target nucleic acid. In some embodiments, a composition or gene editing system of the invention includes a variant ternary complex comprising a variant Cas12i3 polypeptide, an RNA guide, and a target nucleic acid.

In some aspects of the composition or gene editing system, the variant Cas12i3 polypeptide has increased complex formation (e.g., increased ternary complex formation) with the RNA guide and target nucleic acid as compared to a parent polypeptide. In some aspects of the composition or gene editing system, the variant Cas12i3 polypeptide and the RNA guide (e.g., the variant binary complex) have a greater binding affinity to a target nucleic acid, as compared to a parent polypeptide and the RNA guide (e.g., a parent binary complex). In some aspects of the composition or gene editing system, the variant ternary complex is more stable than a parent ternary complex.

In some embodiments, the composition or gene editing system of the present invention includes a variant Cas12i3 polypeptide described herein.

### Variant Effector

In one embodiment, the variant Cas12i3 polypeptide is an isolated or purified polypeptide. In some embodiments, the variant Cas12i3 polypeptide of the present invention is a variant of a parent polypeptide, wherein the parent is encoded by a polynucleotide that comprises a nucleotide sequence such as SEQ ID NO: 1 or SEQ ID NO: 2 or comprises an amino acid sequence such as SEQ ID NO: 3.

TABLE 1
Cas12i3 nucleotide and amino acid sequences.

| Sequence | |
|---|---|
| identifier | |
| SEQ | ATGTCCATCTCTAACAATAACATTCTGCCGTACAACCCGAAGCTGTTGCCGGACGA |
| ID NO: 1 | TCGTAAACACAAGATGCTGGTGGATACCTTCAACCAGCTGGACTTGATTCGCAATA |
| | ACCTGCATGATATGATCATTGCGTTGTACGGTGCGCTGAAGTATGACAACATCAAA |
| | CAATTCGCGAGCAAAGAGAAGCCGCACATTAGCGCTGATGCACTGTGCAGCATCAA |
| | CTGGTTTCGTCTGGTTAAGACCAACGAGCGCAAACCGGCGATTGAATCCAACCAGA |
| | TCATTTCTAAGTTTATCCAATACAGCGGTCACACGCCGGATAAATATGCGCTGAGC |
| | CATATTACCGGCAACCACGAGCCGAGCCATAAGTGGATCGACTGTCGTGAATACGC |
| | GATCAACTATGCCCGCATTATGCACCTGAGCTTCTCCCAGTTTCAAGACTTGGCCA |
| | CCGCGTGCCTGAATTGTAAAATCCTGATTTTGAACGGCACCCTGACCAGCAGCTGG |
| | GCTTGGGGTGCGAACAGCGCACTGTTCGGTGGTAGCGATAAGGAGAACTTTAGCGT |
| | GAAAGCGAAGATCCTGAACAGCTTCATTGAGAACCTGAAAGACGAAATGAACACCA |
| | CGAAGTTTCAGGTGGTTGAAAAAGTTTGCCAGCAAATCGGTAGCAGCGACGCGGCT |
| | GATCTGTTCGACTTGTACCGTAGCACCGTTAAAGATGGTAACCGTGGTCCGGCAAC |
| | CGGTCGTAATCCGAAAGTTATGAACCTGTTTAGCCAGGATGGCGAAATCAGCAGCG |
| | AGCAACGTGAAGACTTCATCGAGAGCTTTCAGAAAGTGATGCAAGAGAAGAACAGC |
| | AAGCAGATCATTCCGCACCTGGATAAGCTGAAGTATCATTTGGTTAAGCAATCCGG |
| | CCTGTACGACATCTATAGCTGGGCAGCCGCGATTAAAAATGCGAACAGCACCATCG |
| | TGGCTAGCAACAGCAGCAACTTGAACACCATCCTGAACAAAACGGAGAAGCAGCAA |
| | ACCTTCGAGGAACTGCGTAAAGACGAAAAGATCGTCGCGTGCTCTAAGATTCTGTT |
| | GTCCGTTAATGATACCTTGCCGGAGGACCTGCACTACAACCCGTCTACCAGCAATT |
| | TGGGTAAAAACCTGGATGTGTTCTTTGACCTGTTGAATGAGAACAGCGTTCATACC |
| | ATTGAAAACAAAGAGGAGAAGAACAAGATCGTTAAAGAATGCGTCAATCAGTACAT |
| | GGAGGAATGTAAAGGCCTGAACAAGCCGCCAATGCCGGTGCTGTTGACCTTCATTA |
| | GCGATTATGCACACAAGCATCAGGCCCAAGACTTCCTGAGCGCTGCAAAGATGAAC |
| | TTCATCGACCTGAAAATCAAGAGCATTAAAGTGGTGCCGACGGTTCACGGTAGCAG |
| | CCCGTACACCTGGATTAGCAATCTGAGCAAGAAGAACAAGGATGGTAAAATGATTC |
| | GTACCCCGAACAGCAGCCTGATCGGCTGGATCATTCCGCCGGAGGAAATCCATGAC |
| | CAGAAGTTCGCAGGCCAAAACCCGATCATTTGGGCCGTGCTGCGTGTTTATTGCAA |
| | TAACAAATGGGAGATGCACCATTTCCCGTTTTCCGACAGCCGTTTCTTCACCGAAG |
| | TGTACGCGTATAAGCCGAATCTGCCGTACTTGCCGGGTGGCGAGAACCGTAGCAAA |
| | CGCTTTGGTTATCGTCACAGCACCAATCTGTCTAACGAAAGCCGCCAGATCCTGCT |
| | GGACAAAAGCAAGTACGCTAAAGCAAACAAGAGCGTTCTGCGTTGCATGGAGAATA |
| | TGACCCATAACGTTGTCTTCGATCCGAAGACCAGCCTGAACATCCGTATTAAAACC |
| | GACAAGAATAACAGCCCGGTGCTGGACGATAAAGGTCGTATCACCTTTGTTATGCA |
| | AATCAACCACCGCATTCTGGAGAAGTACAACAACACCAAGATCGAAATCGGCGATC |
| | GTATTCTGGCGTATGACCAGAATCAAAGCGAGAACCATACGTATGCGATCCTGCAG |
| | CGTACCGAGGAAGGTAGCCACGCTCATCAATTCAACGGCTGGTACGTGCGCGTGCT |
| | GGAAACCGGTAAAGTGACGTCCATTGTTCAGGGTCTGAGCGGCCCGATCGATCAAC |
| | TGAATTATGACGGCATGCCGGTGACCAGCCACAAGTTTAACTGCTGGCAGGCGGAT |
| | CGTAGCGCTTTCGTTTCCCAATTTGCGAGCCTGAAAATTAGCGAAACCGAAACCTT |
| | CGACGAGGCGTACCAGGCGATCAACGCACAAGGTGCCTACACCTGGAACCTGTTTT |
| | ATTTGCGTATTCTGCGCAAGGCGCTGCGTGTTTGCCATATGGAAAATATCAACCAG |
| | TTCCGTGAGGAAATCCTGGCGATTAGCAAAAACCGCCTGTCCCCGATGTCTTTGGG |
| | TAGCCTGTCCCAAAACAGCCTGAAGATGATCCGTGCGTTCAAAAGCATCATTAACT |
| | GTTATATGAGCCGCATGTCCTTTGTGGATGAGCTGCAGAAGAAAGAGGGTGACCTG |
| | GAGCTGCACACGATTATGCGTTTGACCGATAACAAGCTGAACGACAAGCGTGTTGA |
| | GAAAATCAATCGCGCGAGCAGCTTCCTGACCAACAAGGCACATAGCATGGGTTGCA |
| | AAATGATTGTGGGCGAAAGCGATCTGCCGGTTGCGGACTCTAAGACCAGCAAGAAA |
| | CAGAACGTGGATCGTATGGACTGGTGCGCGCGTGCGCTGAGCCACAAGGTTGAGTA |
| | CGCGTGTAAGCTGATGGGTCTGGCATACCGTGGCATCCCGGCCTATATGAGCAGCC |
| | ACCAGGACCCGTTGGTCCATCTGGTGGAATCCAAACGTTCTGTGCTGCGTCCGCGC |
| | TTCGTGGTTGCGGATAAGAGCGACGTTAAACAACACCACCTGGACAATCTGCGTCG |
| | CATGCTGAACAGCAAAACCAAGGTCGGCACCGCGGTGTACTATCGTGAGGCTGTGG |
| | AGCTGATGTGCGAGGAACTGGGTATCCACAAGACCGATATGGCGAAGGGTAAAGTG |
| | AGCCTGAGCGATTTCGTCGACAAGTTTATCGGCGAAAAAGCGATTTTCCCGCAGCG |
| | TGGTGGCCGCTTTTACATGAGCACCAAGCGTTTGACCACGGGTGCAAAACTGATTT |
| | GTTATTCCGGTAGCGACGTTTGGCTGAGCGACGCTGATGAGATCGCCGCGATCAAC |
| | ATTGGCATGTTCGTCGTGTGCGATCAAACCGGCGCGTTCAAGAAAAAGAAAAAGGA |
| | AAAACTGGACGATGAGGAATGTGACATCCTGCCGTTCCGTCCGATG |
| SEQ | ATGTCCATCTCTAACAATAACATTCTGCCCTACAACCCTAAGCTGCTCCCAGACGA |
| ID NO: 2 | TCGAAAACACAAGATGCTGGTGGATACTTTCAATCAGCTGGACCTGATTCGTAATA |
| | ACCTGCATGATATGATCATCGCCCTGTACGGCGCTCTGAAGTATGACAACATCAAA |
| | CAGTTCGCCTCTAAAGAGAAGCCCCACATTTCTGCCGATGCTCTGTGCAGTATCAA |
| | TTGGITTCGACTGGTGAAGACCAACGAGCGTAAACCTGCAATCGAATCCAACCAGA |
| | TCATCTCTAAGTTTATCCAGTACAGCGGACACACTCCCGATAAATATGCTCTGAGC |
| | CATATTACCGGTAACCACGAGCCTTCCCATAAGTGGATCGACTGTCGGGAATACGC |
| | AATCAATTATGCCAGAATTATGCACCTGAGTTTCTCACAGTTTCAGGACCTGGCAA |
| | CAGCCTGCCTGAATTGTAAGATCCTGATCCTGAACGGAACACTGACTAGCTCCTGG |
| | GCATGGGGTGCCAATTCAGCTCTGTTCGGCGGGAGCGATAAGGAGAACTTTTCCGT |
| | GAAAGCCAAGATCCTGAACAGCTTCATTGAGAACCTGAAAGACGAAATGAACACCA |
| | CAAAGTTTCAGGTGGTCGAAAAAGTCTGCCAGCAGATCGGCTCTAGTGACGCCGCT |
| | GATCTGTTCGACCTGTACAGGAGCACAGTGAAGGACGGAAATCGAGGTCCGGCCAC |
| | TGGCCGTAATCCCAAAGTCATGAACCTGTTTTCCCAGGATGGGGAGATTTCAAGCG |
| | AGCAGCGCGAAGACTTCATCGAGTCATTTCAGAAAGTGATGCAGGAGAAGAACAGC |
| | AAGCAGATCATTCCCCACCTGGATAAGCTGAAGTATCACCTGGTCAAGCAGTCAGG |
| | GCTGTACGACATCTATAGCTGGGCAGCCGCCATCAAGAATGCAAACAGTACCATCG |
| | TGGCCTCAAATTCCTCTAACCTGAACACCATCCTGAACAAAACAGAGAAGCAGCAG |
| | ACTTTCGAGGAACTGCGGAAAGACGAAAAGATCGTTGCCTGTAGCAAGATTCTGCT |
| | CTCCGTGAATGATACCCTCCCTGAGGACCTGCACTACAACCCAAGCACATCCAACC |
| | TGGGAAAGAACCTGGATGTCTTCTTTGACCTGCTGAACGAGAACTCCGTTCATACA |
| | ATTGAAAATAAGGAGGAGAAGAACAAGATCGTCAAAGAGTGCGTGAATCAGTACAT |
| | GGAGGAATGTAAAGGCCTGAACAAGCCCCCTATGCCTGTGCTGCTGACCTTCATCA |
| | GCGATTATGCCCACAAGCATCAGGCTCAGGACTTCCTGAGCGCAGCCAAAATGAAC |
| | TTTATCGACCTGAAAATCAAGTCCATTAAGGTTGTGCCGACAGTGCACGGGAGTTC |
| | ACCCTACACTTGGATTAGCAATCTGTCCAAGAAAAACAAAGATGGCAAGATGATTA |
| | GAACACCAAACAGCTCCCTGATCGGGTGGATCATTCCACCGGAGGAAATCCATGAC |
| | CAGAAGTTCGCAGGACAGAATCCTATCATTTGGGCCGTGCTGAGGGTCTATTGCAA |
| | TAACAAATGGGAGATGCACCATTTCCCATTTTCTGATAGTCGCTTCTTTACCGAAG |
| | TGTACGCCTATAAGCCAAATCTGCCGTACCTGCCCGGAGGCGAGAACCGATCCAAG |
| | CGGTTCGGCTATAGGCACTCTACAAATCTGTCTAACGAAAGTCGCCAGATCCTGCT |
| | CGACAAGAGTAAGTACGCTAAAGCAAACAAGTCAGTGCTGCGATGTATGGAGAATA |
| | TGACCCATAACGTGGTGTTCGATCCAAAGACCAGTCTGAATATCCGGATCAAGACA |
| | GACAAGAATAACTCTCCCGTGCTGGACGATAAAGGACGAATCACTTTTGTCATGCA |
| | GATCAACCACCGTATTCTGGAGAAGTACAACAACACCAAGATCGAAATCGGCGATA |
| | GGATTCTGGCCTACGACCAGAATCAGTCTGAGAACCATACTTATGCCATCCTGCAG |
| | CGGACCGAGGAAGGAAGCCACGCTCATCAGTTCAACGGTTGGTACGTGCGGGTGCT |
| | GGAGACCGGAAAGGTGACCTCTATTGTCCAGGGCCTCAGTGGGCCGATCGATCAGC |
| | TGAATTATGACGGTATGCCAGTGACCTCCCACAAGTTTAACTGCTGGCAGGCTGAT |
| | CGGTCTGCATTCGTCAGTCAGTTTGCCTCACTGAAAATTAGCGAGACCGAAACATT |
| | CGACGAGGCATACCAGGCCATCAATGCTCAGGGCGCATACACATGGAACCTGTTCT |
| | ACCTGCGGATTCTGAGAAAGGCTCTGAGAGTGTGCCACATGGAGAATATCAACCAG |
| | TTCCGGGAGGAAATCCTGGCAATTAGCAAGAATAGACTGTCACCCATGAGCCTCGG |
| | GTCACTGAGCCAGAACTCTCTGAAGATGATCAGGGCCTTCAAAAGTATCATTAATT |
| | GTTACATGTCCCGCATGTCTTTTGTGGATGAGCTGCAGAAGAAAGAGGGAGACCTG |
| | GAACTCCACACCATTATGCGGCTGACAGATAACAAGCTGAACGACAAGCGGGTGGA |
| | GAAAATCAATAGAGCTTCTAGTTTTCTGACAAACAAGGCACATTCCATGGGCTGCA |
| | AAATGATCGTGGGGGAATCTGATCTGCCAGTCGCCGACAGTAAGACTTCAAAGAAA |
| | CAGAACGTCGATAGGATGGACTGGTGCGCCCGCGCTCTGAGCCACAAGGTTGAGTA |
| | CGCTTGTAAACTCATGGGACTGGCTTACCGGGGTATCCCTGCATATATGTCAAGCC |
| | ACCAGGATCCACTCGTTCATCTGGTGGAAAGCAAGCGGAGCGTGCTGAGGCCACGT |
| | TTCGTGGTCGCAGATAAGAGCGACGTGAAACAGCACCACCTGGACAATCTGCGGAG |
| | AATGCTGAACTCCAAAACAAAGGTTGGCACTGCCGTGTACTATAGGGAGGCTGTGG |
| | AACTCATGTGCGAGGAACTGGGCATCCACAAGACTGATATGGCCAAAGGGAAGGTC |
| | TCCCTGTCTGATTTCGTTGACAAGTTTATCGGGGAGAAAGCTATTTTCCCTCAGAG |
| | GGGCGGGCGCTTTTACATGTCTACCAAGCGGCTGACTACCGGCGCAAAACTGATTT |
| | GTTATAGTGGGTCAGACGTGTGGCTGAGCGACGCCGATGAGATCGCTGCAATCAAC |
| | ATTGGAATGTTCGTGGTGTGCGATCAGACCGGTGCCTTTAAGAAAAAGAAAAAGGA |
| | AAAGCTCGACGATGAGGAATGTGACATCCTGCCTTTCCGGCCAATG |
| SEQ | MSISNNNILPYNPKLLPDDRKHKMLVDTFNQLDLIRNNLHDMIIALY |
| ID NO: 3 | GALKYDNIKQFASKEKPHISADALCSINWFRLVKTNERKPAIESNQIISKFIQY |
| (Parent) | SGHTPDKYALSHITGNHEPSHKWIDCREYAINYARIMHLSFSQFQDLATACL |
| | NCKILILNGTLTSSWAWGANSALFGGSDKENFSVKAKILNSFIENLKDEMNT |
| | TKFQVVEKVCQQIGSSDAADLFDLYRSTVKDGNRGPATGRNPKVMNLFSQD |
| | GEISSEQREDFIESFQKVMQEKNSKQIIPHLDKLKYHLVKQSGLYDIYSWAAA |
| | IKNANSTIVASNSSNLNTILNKTEKQQTFEELRKDEKIVACSKILLSVNDTLPE |
| | DLHYNPSTSNLGKNLDVFFDLLNENSVHTIENKEEKNKIVKECVNQYMEEC |
| | KGLNKPPMPVLLTFISDYAHKHQAQDFLSAAKMNFIDLKIKSIKVVPTVHGS |
| | SPYTWISNLSKKNKDGKMIRTPNSSLIGWIIPPEEIHDQKFAGQNPIIWAVLRV |
| | YCNNKWEMHHFPFSDSRFFTEVYAYKPNLPYLPGGENRSKRFGYRHSTNLS |
| | NESRQILLDKSKYAKANKSVLRCMENMTHNVVFDPKTSLNIRIKTDKNNSPV |
| | LDDKGRITFVMQINHRILEKYNNTKIEIGDRILAYDQNQSENHTYAILQRTEE |
| | GSHAHQFNGWYVRVLETGKVTSIVQGLSGPIDQLNYDGMPVTSHKFNCWQ |
| | ADRSAFVSQFASLKISETETFDEAYQAINAQGAYTWNLFYLRILRKALRVCH |
| | MENINQFREEILAISKNRLSPMSLGSLSQNSLKMIRAFKSIINCYMSRMSFVDE |
| | LQKKEGDLELHTIMRLTDNKLNDKRVEKINRASSFLTNKAHSMGCKMIVGE |
| | SDLPVADSKTSKKQNVDRMDWCARALSHKVEYACKLMGLAYRGIPAYMSS |
| | HQDPLVHLVESKRSVLRPRFVVADKSDVKQHHLDNLRRMLNSKTKVGTAV |
| | YYREAVELMCEELGIHKTDMAKGKVSLSDFVDKFIGEKAIFPQRGGRFYMS |
| | TKRLTTGAKLICYSGSDVWLSDADEIAAINIGMFVVCDQTGAFKKKKKEKL |
| | DDEECDILPFRPM |
| SEQ | MSISNNNILPYNPKLLPDDRKHKMLVDTFNQLDLIRNNLHDMIIALY |
| ID NO: 7 | GALKYDNIKQFASKEKPHISADALCSINWFRLVKTNERKPAIESNQIISKFIQY |
| (Variant) | SGHTPDKYALSHITGNHEPSHKWIDCREYAINYARIMHLSFSQFQDLATACL |
| | NCKILILNGTLTSSWAWGANSALFGGSDKENFSVKAKILNSFIENLKDEMNT |
| | TKFQVVEKVCQQIGSSDAADLFDLYRSTVKDGNRGPATGRNPKVMNLFSQD |
| | GEISSEQREDFIESFQKVMQEKNSKQIIPHLDKLKYHLVKQSGLYDIYSWAAA |
| | IKNANSTIVASNSSNLNTILNKTEKQQTFEELRKDEKIVACSKILLSVNDTLPE |
| | DLHYNPSTSNLGKNLDVFFDLLNENSVHTIENKEEKNKIVKECVNQYMEEC |
| | KGLNKPPMPVLLTFISDYAHKHQAQDFLSAAKMNFIDLKIKSIKVVPTVHGS |
| | SPYTWISNLSKKNKDGKMIRTPNSSLIGWIIPPEEIHDQKFAGQNPIIWAVLRV |
| | YCNNKWEMHHFPFSDSRFFTEVYAYKPNLPYLPGGENRSKRFGYRHSTNLS |
| | NESRQILLDKSRYAKANKSVLRCMENMTHNVVFDPKTSLNIRIKTDKNNSPV |
| | LDDKGRITFVMQINHRILEKYNNTKIEIGDRILAYDQNQSENHTYAILQRTEE |
| | GSHAHQFNGWYVRVLETGKVTSIVQGLSGPIDQLRYDGMPVTSHKFNRWQ |
| | ADRSAFVSQFASLKISETETFDEAYQAINAQGAYTWNLFYLRILRKALRVCH |
| | MENINQFREEILAISKNRLSPMSLGSLSQNSLKMIRAFKSIINCYMSRMSFVDE |
| | LQKKEGDLELHTIMRLTDNKLNDKRVEKINRASSFLINKAHSMGCKMIVGE |
| | SDLPVADSKTSKKQNVDRMDWCARALSHKVEYACKLMGLAYRGIPAYMSS |
| | HQDPLVHLVESKRSVLRPRFVVADKSDVKQHHLDNLRRMLNSKTKVGTAV |
| | YYREAVELMCEELGIHKTDMAKGKVSLSDFVDKFIGEKAIFPQRGGRFYMS |
| | TKRLTTGAKLICYSGSDVWLSDADEIAAINIGMFVVCDQTGAFKKKKKEKL |
| | DDEECDILPFRPM |

A nucleic acid sequence encoding the parent polypeptide described herein may be substantially identical to a reference nucleic acid sequence, e.g., SEQ ID NO: 1 or SEQ ID NO: 2. In some embodiments, the variant Cas12i3 polypeptide is encoded by a nucleic acid comprising a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence, e.g., nucleic acid sequence encoding the parent polypeptide, e.g., SEQ ID NO: 1 or SEQ ID NO: 2. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the nucleic acid molecules hybridize to the complementary sequence of the other under stringent conditions (e.g., within a range of medium to high stringency).

In some embodiments, the variant Cas12i3 polypeptide is encoded by a nucleic acid sequence having at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity, but not 100% sequence identity, to a reference nucleic acid sequence, e.g., nucleic acid sequence encoding the parent polypeptide, e.g., SEQ ID NO: 1 or SEQ ID NO: 2.

In some embodiments, the variant Cas12i3 polypeptide of the present invention comprises a polypeptide sequence having 50%, 60%, 65%, 70%, 75%, 80%, 85%, 90%, 91%, 92%, 93%, 94%, 95%, 96%, 97%, 98%, or 99%, but not 100%, identity to SEQ ID NO: 3. In some embodiments, the variant Cas12i3 polypeptide of the present invention comprises a polypeptide sequence having greater than 50%. 60%, 65%, 70%, 75%, 80%, 85%, 90%, 91%, 92%, 93%, 94%, 95%, 96%, 97%, 98%, or 99%, but not 100%, identity to SEQ ID NO: 3.

In some embodiments, the present invention describes a variant Cas12i3 polypeptide having a specified degree of amino acid sequence identity to one or more reference polypeptides, e.g., a parent polypeptide, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%, but not 100%, sequence identity to the amino acid sequence of SEQ ID NO: 3. Homology or identity can be determined by amino acid sequence alignment, e.g., using a program such as BLAST, ALIGN, or CLUSTAL, as described herein.

Also provided is a variant Cas12i3 polypeptide of the present invention having enzymatic activity, e.g., nuclease or endonuclease activity, and comprising an amino acid sequence which differs from the amino acid sequences of any one of a parent polypeptide and SEQ ID NO: 3 by 50, 40, 35, 30, 25, 20, 19, 18, 17, 16, 15, 14, 13, 12, 11, 10, 9, 8, 7, 6, 5, 4, 3, 2, or 1 amino acid residue(s), when aligned using any of the previously described alignment methods.

In some embodiments, the variant Cas12i3 polypeptide comprises an alteration at one or more (e.g., several) amino acids of a parent polypeptide, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more are altered.

In some embodiments, the variant Cas12i3 polypeptide comprises one or more of the amino acid substitutions listed in Table 2.

### Table 2. Single Amino Acid Substitutions in Variants of SEQ ID NO: 3.

TABLE 2

| | Wild-Type | |
|---|---|---|
| Position | Residue | Substitution(s) |
| 1 | M | R, G, A, K, Q, N, H |
| 2 | S | R, G, A, K, Q, N, H |
| 3 | I | R, G, A, K, Q, N, H |
| 4 | S | R, G, A, K, Q, N, H |
| 5 | N | R, G, A, K, Q, H |
| 6 | N | R, G, A, K, Q, H |
| 7 | N | R, G, A, K, Q, H |
| 8 | I | R, G, A, K, Q, N, H |
| 9 | L | R, G, A, K, Q, N, H |
| 10 | P | R, G, A, K, Q, N, H |
| 11 | Y | R, G, A, K, Q, N, H |
| 12 | N | R, G, A, K, Q, H |
| 13 | P | R, G, A, K, Q, N, H |
| 14 | K | R, G, A, Q, N, H |
| 15 | L | R, G, A, K, Q, N, H |
| 16 | L | R, G, A, K, Q, N, H |
| 17 | P | R, G, A, K, Q, N, H |
| 18 | D | R, G, A, K, Q, N, H |
| 19 | D | R, G, A, K, Q, N, H |
| 20 | R | G, A, K, Q, N, H |
| 21 | K | R, G, A, Q, N, H |
| 22 | H | R, G, A, K, Q, N |
| 23 | K | R, G, A, Q, N, H |
| 24 | M | R, G, A, K, Q, N, H |
| 25 | L | R, G, A, K, Q, N, H |
| 26 | V | R, G, A, K, Q, N, H |
| 27 | D | R, G, A, K, Q, N, H |
| 28 | T | R, G, A, K, Q, N, H |
| 29 | F | R, G, A, K, Q, N, H |
| 30 | N | R, G, A, K, Q, H |
| 31 | Q | R, G, A, K, N, H |
| 32 | L | R, G, A, K, Q, N, H |
| 33 | D | R, G, A, K, Q, N, H |
| 34 | L | R, G, A, K, Q, N, H |
| 35 | I | R, G, A, K, Q, N, H |
| 36 | R | G, A, K, Q, N, H |
| 37 | N | R, G, A, K, Q, H |
| 38 | N | R, G, A, K, Q, H |
| 39 | L | R, G, A, K, Q, N, H |
| 40 | H | R, G, A, K, Q, N |
| 41 | D | R, G, A, K, Q, N, H |
| 42 | M | R, G, A, K, Q, N, H |
| 43 | I | R, G, A, K, Q, N, H |
| 44 | I | R, G, A, K, Q, N, H |
| 45 | A | R, G, K, Q, N, H |
| 46 | L | R, G, A, K, Q, N, H |
| 47 | Y | R, G, A, K, Q, N, H |
| 48 | G | R, A, K, Q, N, H |
| 49 | A | R, G, K, Q, N, H |
| 50 | L | R, G, A, K, Q, N, H |
| 51 | K | R, G, A, Q, N, H |
| 52 | Y | R, G, A, K, Q, N, H |
| 53 | D | R, G, A, K, Q, N, H |
| 54 | N | R, G, A, K, Q, H |
| 55 | I | R, G, A, K, Q, N, H |
| 56 | K | R, G, A, Q, N, H |
| 57 | Q | R, G, A, K, N, H |
| 58 | F | R, G, A, K, Q, N, H |
| 59 | A | R, G, K, Q, N, H |
| 60 | S | R, G, A, K, Q, N, H |
| 61 | K | R, G, A, Q, N, H |
| 62 | E | R, G, A, K, Q, N, H |
| 63 | K | R, G, A, Q, N, H |
| 64 | P | R, G, A, K, Q, N, H |
| 65 | H | R, G, A, K, Q, N |
| 66 | I | R, G, A, K, Q, N, H |
| 67 | S | R, G, A, K, Q, N, H |
| 68 | A | R, G, K, Q, N, H |
| 69 | D | R, G, A, K, Q, N, H |
| 70 | A | R, G, K, Q, N, H |
| 71 | L | R, G, A, K, Q, N, H |
| 72 | C | R, G, A, K, Q, N, H |
| 73 | S | R, G, A, K, Q, N, H |
| 74 | I | R, G, A, K, Q, N, H |
| 75 | N | R, G, A, K, Q, H |
| 76 | W | R, G, A, K, Q, N, H |
| 77 | F | R, G, A, K, Q, N, H |
| 78 | R | G, A, K, Q, N, H |
| 79 | L | R, G, A, K, Q, N, H |
| 80 | V | R, G, A, K, Q, N, H |
| 81 | K | R, G, A, Q, N, H |
| 82 | T | R, G, A, K, Q, N, H |
| 83 | N | R, G, A, K, Q, H |
| 84 | E | R, G, A, K, Q, N, H |
| 85 | R | G, A, K, Q, N, H |
| 86 | K | R, G, A, Q, N, H |
| 87 | P | R, G, A, K, Q, N, H |
| 88 | A | R, G, K, Q, N, H |
| 89 | I | R, G, A, K, Q, N, H |
| 90 | E | R, G, A, K, Q, N, H |
| 91 | S | R, G, A, K, Q, N, H |
| 92 | N | R, G, A, K, Q, H |
| 93 | Q | R, G, A, K, N, H |
| 94 | I | R, G, A, K, Q, N, H |
| 95 | I | R, G, A, K, Q, N, H |
| 96 | S | R, G, A, K, Q, N, H |
| 97 | K | R, G, A, Q, N, H |
| 98 | F | R, G, A, K, Q, N, H |
| 99 | I | R, G, A, K, Q, N, H |
| 100 | Q | R, G, A, K, N, H |
| 101 | Y | R, G, A, K, Q, N, H |
| 102 | S | R, G, A, K, Q, N, H |
| 103 | G | R, A, K, Q, N, H |
| 104 | H | R, G, A, K, Q, N |
| 105 | T | R, G, A, K, Q, N, H |
| 106 | P | R, G, A, K, Q, N, H |
| 107 | D | R, G, A, K, Q, N, H |
| 108 | K | R, G, A, Q, N, H |
| 109 | Y | R, G, A, K, Q, N, H |
| 110 | A | R, G, K, Q, N, H |
| 111 | L | R, G, A, K, Q, N, H |
| 112 | S | R, G, A, K, Q, N, H |
| 113 | H | R, G, A, K, Q, N |
| 114 | I | R, G, A, K, Q, N, H |
| 115 | T | R, G, A, K, Q, N, H |
| 116 | G | R, A, K, Q, N, H |
| 117 | N | R, G, A, K, Q, N, H |
| 118 | H | R, G, A, Q, N, H |
| 119 | E | R, G, A, K, Q, N, H |
| 120 | P | R, G, A, K, Q, N, H |
| 121 | S | R, G, A, K, Q, N, H |
| 122 | H | R, G, A, K, Q, N, H |
| 123 | K | R, G, A, K, Q, H |
| 124 | W | R, A, K, Q, N, H |
| 125 | I | R, G, A, K, Q, N, H |
| 126 | D | R, G, A, K, Q, N, H |
| 127 | C | R, G, A, K, Q, N, H |
| 128 | R | R, G, A, K, Q, N, H |
| 129 | E | R, G, A, K, Q, N, H |
| 130 | Y | R, G, A, K, Q, N, H |
| 131 | A | R, G, K, Q, N, H |
| 132 | I | R, G, A, K, Q, N, H |
| 133 | N | R, A, K, Q, N, H |
| 134 | Y | R, G, K, Q, N, H |
| 135 | A | R, G, A, K, Q, H |
| 136 | R | R, G, A, K, Q, N, H |
| 137 | I | R, G, K, Q, N, H |
| 138 | M | R, G, A, K, Q, N, H |
| 139 | H | R, G, A, K, Q, N, H |
| 140 | L | R, A, K, Q, N, H |
| 141 | S | R, A, K, Q, N, H |
| 142 | F | R, G, A, K, Q, N, H |
| 143 | S | R, G, A, K, Q, N, H |
| 144 | Q | R, G, A, Q, N, H |
| 145 | F | R, G, A, K, Q, N, H |
| 146 | Q | R, G, A, K, Q, H |
| 147 | D | R, G, A, K, Q, N, H |
| 148 | L | R, G, A, K, Q, N, H |
| 149 | A | R, G, A, K, Q, N, H |
| 150 | T | R, G, A, Q, N, H |
| 151 | A | R, G, K, Q, N, H |
| 152 | C | R, G, A, Q, N, H |
| 153 | L | R, G, A, K, Q, N, H |
| 154 | N | R, G, A, K, Q, N, H |
| 155 | C | R, G, A, K, Q, N, H |
| 156 | K | R, G, A, Q, N, H |
| 157 | I | R, G, A, K, Q, N, H |
| 158 | L | R, G, A, K, Q, N, H |
| 159 | I | R, G, A, K, Q, N, H |
| 160 | L | R, G, A, K, Q, N, H |
| 161 | N | R, G, A, K, Q, H |
| 162 | G | R, A, K, Q, N, H |
| 163 | T | R, G, A, K, Q, N, H |
| 164 | L | R, G, A, K, Q, N, H |
| 165 | T | R, G, A, K, Q, N, H |
| 166 | S | R, G, A, K, Q, N, H |
| 167 | S | R, G, A, K, Q, N, H |
| 168 | W | R, G, A, K, Q, N, H |
| 169 | A | R, G, K, Q, N, H |
| 170 | W | R, G, A, K, Q, N, H |
| 171 | G | R, A, K, Q, N, H |
| 172 | A | R, G, K, Q, N, H |
| 173 | N | R, G, A, K, Q, H |
| 174 | S | R, G, A, K, Q, N, H |
| 175 | A | R, G, K, Q, N, H |
| 176 | L | R, G, A, K, Q, N, H |
| 177 | F | R, G, A, K, Q, N, H |
| 178 | G | R, A, K, Q, N, H |
| 179 | G | R, A, K, Q, N, H |
| 180 | S | R, G, A, K, Q, N, H |
| 181 | D | R, G, A, K, Q, N, H |
| 182 | K | R, G, A, Q, N, H |
| 183 | E | R, G, A, K, Q, N, H |
| 184 | N | R, G, A, K, Q, H |
| 185 | F | R, G, A, K, Q, N, H |
| 186 | S | R, G, A, K, Q, N, H |
| 187 | V | R, G, A, K, Q, N, H |
| 188 | K | R, G, A, Q, N, H |
| 189 | A | R, G, K, Q, N, H |
| 190 | K | R, G, A, Q, N, H |
| 191 | I | R, G, A, K, Q, N, H |
| 192 | L | R, G, A, K, Q, N, H |
| 193 | N | R, G, A, K, Q, H |
| 194 | S | R, G, A, K, Q, N, H |
| 195 | F | R, G, A, K, Q, N, H |
| 196 | I | R, G, A, K, Q, N, H |
| 197 | E | R, G, A, K, Q, N, H |
| 198 | N | R, G, A, K, Q, H |
| 199 | L | R, G, A, K, Q, N, H |
| 200 | K | R, G, A, Q, N, H |
| 201 | D | R, G, A, K, Q, N, H |
| 202 | E | R, G, A, K, Q, N, H |
| 203 | M | R, G, A, K, Q, N, H |
| 204 | N | R, G, A, K, Q, H |
| 205 | T | R, G, A, K, Q, N, H |
| 206 | T | R, G, A, K, Q, N, H |
| 207 | K | R, G, A, Q, N, H |
| 208 | F | R, G, A, K, Q, N, H |
| 209 | Q | R, G, A, K, N, H |
| 210 | V | R, G, A, K, Q, N, H |
| 211 | V | R, G, A, K, Q, N, H |
| 212 | E | R, G, A, K, Q, N, H |
| 213 | K | R, G, A, Q, N, H |
| 214 | V | R, G, A, K, Q, N, H |
| 215 | C | R, G, A, K, Q, N, H |
| 216 | Q | R, G, A, K, N, H |
| 217 | Q | R, G, A, K, N, H |
| 218 | I | R, G, A, K, Q, N, H |
| 219 | G | R, A, K, Q, N, H |
| 220 | S | R, G, A, K, Q, N, H |
| 221 | S | R, G, A, K, Q, N, H |
| 222 | D | R, G, A, K, Q, N, H |
| 223 | A | R, G, K, Q, N, H |
| 224 | A | R, G, K, Q, N, H |
| 225 | D | R, G, A, K, Q, N, H |
| 226 | L | R, G, A, K, Q, N, H |
| 227 | F | R, G, A, K, Q, N, H |
| 228 | D | R, G, A, K, Q, N, H |
| 229 | L | R, G, A, K, Q, N, H |
| 230 | Y | R, G, A, K, Q, N, H |
| 231 | R | G, A, K, Q, N, H |
| 232 | S | R, G, A, K, Q, N, H |
| 233 | T | R, G, A, K, Q, N, H |
| 234 | V | R, G, A, K, Q, N, H |
| 235 | K | R, G, A, Q, N, H |
| 236 | D | R, G, A, K, Q, N, H |
| 237 | G | R, A, K, Q, N, H |
| 238 | N | R, G, A, K, Q, H |
| 239 | R | G, A, K, Q, N, H |
| 240 | G | R, A, K, Q, N, H |
| 241 | P | R, G, A, K, Q, N, H |
| 242 | A | R, G, K, Q, N, H |
| 243 | T | R, G, A, K, Q, N, H |
| 244 | G | R, A, K, Q, N, H |
| 245 | R | G, A, K, Q, N, H |
| 246 | N | R, G, A, K, Q, H |
| 247 | E | R, G, A, K, Q, N, H |
| 248 | K | R, G, A, Q, N, H |
| 249 | V | R, G, A, K, Q, N, H |
| 250 | M | R, G, A, K, Q, N, H |
| 251 | N | R, G, A, K, Q, H |
| 252 | L | R, G, A, K, Q, N, H |
| 253 | F | R, G, A, K, Q, N, H |
| 254 | S | R, G, A, K, Q, N, H |
| 255 | Q | R, G, A, K, N, H |
| 256 | D | R, G, A, K, Q, N, H |
| 257 | G | R, A, K, Q, N, H |
| 258 | E | R, G, A, K, Q, N, H |
| 259 | I | R, G, A, K, Q, N, H |
| 260 | S | R, G, A, K, Q, N, H |
| 261 | S | R, G, A, K, Q, N, H |
| 262 | E | R, G, A, K, Q, N, H |
| 263 | Q | R, G, A, K, N, H |
| 264 | R | G, A, K, Q, N, H |
| 265 | E | R, G, A, K, Q, N, H |
| 266 | D | R, G, A, K, Q, N, H |
| 267 | F | R, G, A, K, Q, N, H |
| 268 | I | R, G, A, K, Q, N, H |
| 269 | E | R, G, A, K, Q, N, H |
| 270 | S | R, G, A, K, Q, N, H |
| 271 | F | R, G, A, K, Q, N, H |
| 272 | Q | R, G, A, K, N, H |
| 273 | K | R, G, A, Q, N, H |
| 274 | V | R, G, A, K, Q, N, H |
| 275 | M | R, G, A, K, Q, N, H |
| 276 | Q | R, G, A, K, N, H |
| 277 | E | R, G, A, K, Q, N, H |
| 278 | K | R, G, A, Q, N, H |
| 279 | N | R, G, A, K, Q, H |
| 280 | S | R, G, A, K, Q, N, H |
| 281 | K | R, G, A, Q, N, H |
| 282 | Q | R, G, A, K, N, H |
| 283 | I | R, G, A, K, Q, N, H |
| 284 | I | R, G, A, K, Q, N, H |
| 285 | P | R, G, A, K, Q, N, H |
| 286 | H | R, G, A, K, Q, N |
| 287 | L | R, G, A, K, Q, N, H |
| 288 | D | R, G, A, K, Q, N, H |
| 289 | K | R, G, A, Q, N, H |
| 290 | L | R, G, A, K, Q, N, H |
| 291 | K | R, G, A, Q, N, H |
| 292 | Y | R, G, A, K, Q, N, H |
| 293 | H | R, G, A, K, Q, N |
| 294 | L | R, G, A, K, Q, N, H |
| 295 | V | R, G, A, K, Q, N, H |
| 296 | K | R, G, A, Q, N, H |
| 297 | Q | R, G, A, K, N, H |
| 298 | S | R, G, A, K, Q, N, H |
| 299 | G | R, A, K, Q, N, H |
| 300 | L | R, G, A, K, Q, N, H |
| 301 | Y | R, G, A, K, Q, N, H |
| 302 | D | R, G, A, K, Q, N, H |
| 303 | I | R, G, A, K, Q, N, H |
| 304 | Y | R, G, A, K, Q, N, H |
| 305 | S | R, G, A, K, Q, N, H |
| 306 | W | R, G, A, K, Q, N, H |
| 307 | A | R, G, K, Q, N, H |
| 308 | A | R, G, K, Q, N, H |
| 309 | A | R, G, K, Q, N, H |
| 310 | I | R, G, A, K, Q, N, H |
| 311 | K | R, G, A, Q, N, H |
| 312 | N | R, G, A, K, Q, H |
| 313 | A | R, G, K, Q, N, H |
| 314 | N | R, G, A, K, Q, H |
| 315 | S | R, G, A, K, Q, N, H |
| 316 | T | R, G, A, K, Q, N, H |
| 317 | I | R, G, A, K, Q, N, H |
| 318 | V | R, G, A, K, Q, N, H |
| 319 | A | R, G, K, Q, N, H |
| 320 | S | R, G, A, K, Q, N, H |
| 321 | N | R, G, A, K, Q, H |
| 322 | S | R, G, A, K, Q, N, H |
| 323 | S | R, G, A, K, Q, N, H |
| 324 | N | R, G, A, K, Q, H |
| 325 | L | R, G, A, K, Q, N, H |
| 326 | N | R, G, A, K, Q, H |
| 327 | T | R, G, A, K, Q, N, H |
| 328 | I | R, G, A, K, Q, N, H |
| 329 | L | R, G, A, K, Q, N, H |
| 330 | N | R, G, A, K, Q, H |
| 331 | K | R, G, A, Q, N, H |
| 332 | T | R, G, A, K, Q, N, H |
| 333 | E | R, G, A, K, Q, N, H |
| 334 | K | R, G, A, Q, N, H |
| 335 | Q | R, G, A, K, N, H |
| 336 | Q | R, G, A, K, N, H |
| 337 | T | R, G, A, K, Q, N, H |
| 338 | F | R, G, A, K, Q, N, H |
| 339 | E | R, G, A, K, Q, N, H |
| 340 | E | R, G, A, K, Q, N, H |
| 341 | L | R, G, A, K, Q, N, H |
| 342 | R | G, A, K, Q, N, H |
| 343 | K | R, G, A, Q, N, H |
| 344 | D | R, G, A, K, Q, N, H |
| 345 | E | R, G, A, K, Q, N, H |
| 346 | K | R, G, A, Q, N, H |
| 347 | I | R, G, A, K, Q, N, H |
| 348 | V | R, G, A, K, Q, N, H |
| 349 | A | R, G, K, Q, N, H |
| 350 | C | R, G, A, K, Q, N, H |
| 351 | S | R, G, A, K, Q, N, H |
| 352 | K | R, G, A, Q, N, H |
| 353 | I | R, G, A, K, Q, N, H |
| 354 | L | R, G, A, K, Q, N, H |
| 355 | L | R, G, A, K, Q, N, H |
| 356 | S | R, G, A, K, Q, N, H |
| 357 | V | R, G, A, K, Q, N, H |
| 358 | N | R, G, A, K, Q, H |
| 359 | D | R, G, A, K, Q, N, H |
| 360 | T | R, G, A, K, Q, N, H |
| 361 | L | R, G, A, K, Q, N, H |
| 362 | P | R, G, A, K, Q, N, H |
| 363 | E | R, G, A, K, Q, N, H |
| 364 | D | R, G, A, K, Q, N, H |
| 365 | L | R, G, A, K, Q, N, H |
| 366 | H | R, G, A, K, Q, N |
| 367 | Y | R, G, A, K, Q, N, H |
| 368 | N | R, G, A, K, Q, H |
| 369 | P | R, G, A, K, Q, N, H |
| 370 | S | R, G, A, K, Q, N, H |
| 371 | T | R, G, A, K, Q, N, H |
| 372 | S | R, G, A, K, Q, N, H |
| 373 | N | R, G, A, K, Q, H |
| 374 | L | R, G, A, K, Q, N, H |
| 375 | G | R, A, K, Q, N, H |
| 376 | K | R, G, A, Q, N, H |
| 377 | N | R, G, A, K, Q, H |
| 378 | L | R, G, A, K, Q, N, H |
| 379 | D | R, G, A, K, Q, N, H |
| 380 | V | R, G, A, K, Q, N, H |
| 381 | F | R, G, A, K, Q, N, H |
| 382 | F | R, G, A, K, Q, N, H |
| 383 | D | R, G, A, K, Q, N, H |
| 384 | L | R, G, A, K, Q, N, H |
| 385 | L | R, G, A, K, Q, N, H |
| 386 | N | R, G, A, K, Q, H |
| 387 | E | R, G, A, K, Q, N, H |
| 388 | N | R, G, A, K, Q, H |
| 389 | S | R, G, A, K, Q, N, H |
| 390 | V | R, G, A, K, Q, N, H |
| 391 | H | R, G, A, K, Q, N |
| 392 | T | R, G, A, K, Q, N, H |
| 393 | I | R, G, A, K, Q, N, H |
| 394 | E | R, G, A, K, Q, N, H |
| 395 | N | R, G, A, K, Q, H |
| 396 | K | R, G, A, Q, N, H |
| 397 | E | R, G, A, K, Q, N, H |
| 398 | E | R, G, A, K, Q, N, H |
| 399 | K | R, G, A, Q, N, H |
| 400 | N | R, G, A, K, Q, H |
| 401 | K | R, G, A, Q, N, H |
| 402 | I | R, G, A, K, Q, N, H |
| 403 | V | R, G, A, K, Q, N, H |
| 404 | K | R, G, A, Q, N, H |
| 405 | E | R, G, A, K, Q, N, H |
| 406 | C | R, G, A, K, Q, N, H |
| 407 | V | R, G, A, K, Q, N, H |
| 408 | N | R, G, A, K, Q, H |
| 409 | Q | R, G, A, K, N, H |
| 410 | Y | R, G, A, K, Q, N, H |
| 411 | M | R, G, A, K, Q, N, H |
| 412 | E | R, G, A, K, Q, N, H |
| 413 | E | R, G, A, K, Q, N, H |
| 414 | C | R, G, A, K, Q, N, H |
| 415 | K | R, G, A, Q, N, H |
| 416 | G | R, A, K, Q, N, H |
| 417 | L | R, G, A, K, Q, N, H |
| 418 | N | R, G, A, K, Q, H |
| 419 | K | R, G, A, Q, N, H |
| 420 | P | R, G, A, K, Q, N, H |
| 421 | P | R, G, A, K, Q, N, H |
| 422 | M | R, G, A, K, Q, N, H |
| 423 | P | R, G, A, K, Q, N, H |
| 424 | V | R, G, A, K, Q, N, H |
| 425 | L | R, G, A, K, Q, N, H |
| 426 | L | R, G, A, K, Q, N, H |
| 427 | T | R, G, A, K, Q, N, H |
| 428 | F | R, G, A, K, Q, N, H |
| 429 | I | R, G, A, K, Q, N, H |
| 430 | S | R, G, A, K, Q, N, H |
| 431 | D | R, G, A, K, Q, N, H |
| 432 | Y | R, G, A, K, Q, N, H |
| 433 | A | R, G, K, Q, N, H |
| 434 | H | R, G, A, K, Q, N |
| 435 | K | R, G, A, Q, N, H |
| 436 | H | R, G, A, K, Q, N |
| 437 | Q | R, G, A, K, N, H |
| 438 | A | R, G, K, Q, N, H |
| 439 | Q | R, G, A, K, N, H |
| 440 | D | R, G, A, K, Q, N, H |
| 441 | F | R, G, A, K, Q, N, H |
| 442 | L | R, G, A, K, Q, N, H |
| 443 | S | R, G, A, K, Q, N, H |
| 444 | A | R, G, K, Q, N, H |
| 445 | A | R, G, K, Q, N, H |
| 446 | K | R, G, A, Q, N, H |
| 447 | M | R, G, A, K, Q, N, H |
| 448 | N | R, G, A, K, Q, H |
| 449 | F | R, G, A, K, Q, N, H |
| 450 | I | R, G, A, K, Q, N, H |
| 451 | D | R, G, A, K, Q, N, H |
| 452 | L | R, G, A, K, Q, N, H |
| 453 | K | R, G, A, Q, N, H |
| 454 | I | R, G, A, K, Q, N, H |
| 455 | K | R, G, A, Q, N, H |
| 456 | S | R, G, A, K, Q, N, H |
| 457 | I | R, G, A, K, Q, N, H |
| 458 | K | R, G, A, Q, N, H |
| 459 | V | R, G, A, K, Q, N, H |
| 460 | V | R, G, A, K, Q, N, H |
| 461 | P | R, G, A, K, Q, N, H |
| 462 | T | R, G, A, K, Q, N, H |
| 463 | V | R, G, A, K, Q, N, H |
| 464 | H | R, G, A, K, Q, N |
| 465 | G | R, A, K, Q, N, H |
| 466 | S | R, G, A, K, Q, N, H |
| 467 | S | R, G, A, K, Q, N, H |
| 468 | P | R, G, A, K, Q, N, H |
| 469 | Y | R, G, A, K, Q, N, H |
| 470 | T | R, G, A, K, Q, N, H |
| 471 | W | R, G, A, K, Q, N, H |
| 472 | I | R, G, A, K, Q, N, H |
| 473 | S | R, G, A, K, Q, N, H |
| 474 | N | R, G, A, K, Q, N, H |
| 475 | L | R, G, A, K, Q, N, H |
| 476 | S | R, G, A, K, Q, N, H |
| 477 | K | R, G, A, Q, N, H |
| 478 | K | R, G, A, Q, N, H |
| 479 | N | R, G, A, K, Q, H |
| 480 | K | R, G, A, Q, N, H |
| 481 | D | R, G, A, K, Q, N, H |
| 482 | G | R, A, K, Q, N, H |
| 483 | K | R, G, A, Q, N, H |
| 484 | M | R, G, A, K, Q, N, H |
| 485 | I | R, G, A, K, Q, N, H |
| 486 | R | G, A, K, Q, N, H |
| 487 | T | R, G, A, K, Q, N, H |
| 488 | P | R, G, A, K, Q, N, H |
| 489 | N | R, G, A, K, Q, H |
| 490 | S | R, G, A, K, Q, N, H |
| 491 | S | R, G, A, K, Q, N, H |
| 492 | L | R, G, A, K, Q, N, H |
| 493 | I | R, G, A, K, Q, N, H |
| 494 | G | R, A, K, Q, N, H |
| 495 | W | R, G, A, K, Q, N, H |
| 496 | I | R, G, A, K, Q, N, H |
| 497 | I | R, G, A, K, Q, N, H |
| 498 | P | R, G, A, K, Q, N, H |
| 499 | P | R, G, A, K, Q, N, H |
| 500 | E | R, G, A, K, Q, N, H |
| 501 | E | R, G, A, K, Q, N, H |
| 502 | I | R, G, A, K, Q, N, H |
| 503 | H | R, G, A, K, Q, N |
| 504 | D | R, G, A, K, Q, N, H |
| 505 | Q | R, G, A, K, N, H |
| 506 | K | R, G, A, Q, N, H |
| 507 | F | R, G, A, K, Q, N, H |
| 508 | A | R, G, K, Q, N, H |
| 509 | G | R, A, K, Q, N, H |
| 510 | Q | R, G, A, K, N, H |
| 511 | N | R, G, A, K, Q, H |
| 512 | P | R, G, A, K, Q, N, H |
| 513 | I | R, G, A, K, Q, N, H |
| 514 | I | R, G, A, K, Q, N, H |
| 515 | W | R, G, A, K, Q, N, H |
| 516 | A | R, G, K, Q, N, H |
| 517 | V | R, G, A, K, Q, N, H |
| 518 | L | R, G, A, K, Q, N, H |
| 519 | R | G, A, K, Q, N, H |
| 520 | V | R, G, A, K, Q, N, H |
| 521 | Y | R, G, A, K, Q, N, H |
| 522 | C | R, G, A, K, Q, N, H |
| 523 | N | R, G, A, K, Q, H |
| 524 | N | R, G, A, K, Q, H |
| 525 | K | R, G, A, Q, N, H |
| 526 | W | R, G, A, K, Q, N, H |
| 527 | E | R, G, A, K, Q, N, H |
| 528 | M | R, G, A, K, Q, N, H |
| 529 | H | R, G, A, K, Q, N |
| 530 | H | R, G, A, K, Q, N |
| 531 | F | R, G, A, K, Q, N, H |
| 532 | P | R, G, A, K, Q, N, H |
| 533 | F | R, G, A, K, Q, N, H |
| 534 | S | R, G, A, K, Q, N, H |
| 535 | D | R, G, A, K, Q, N, H |
| 536 | S | R, G, A, K, Q, N, H |
| 537 | R | G, A, K, Q, N, H |
| 538 | F | R, G, A, K, Q, N, H |
| 539 | F | R, G, A, K, Q, N, H |
| 540 | T | R, G, A, K, Q, N, H |
| 541 | E | R, G, A, K, Q, N, H |
| 542 | V | R, G, A, K, Q, N, H |
| 543 | Y | R, G, A, K, Q, N, H |
| 544 | A | R, G, K, Q, N, H |
| 545 | Y | R, G, A, K, Q, N, H |
| 546 | K | R, G, A, Q, N, H |
| 547 | P | R, G, A, K, Q, N, H |
| 548 | N | R, G, A, K, Q, H |
| 549 | L | R, G, A, K, Q, N, H |
| 550 | P | R, G, A, K, Q, N, H |
| 551 | Y | R, G, A, K, Q, N, H |
| 552 | L | R, G, A, K, Q, N, H |
| 553 | P | R, G, A, K, Q, N, H |
| 554 | G | R, A, K, Q, N, H |
| 555 | G | R, A, K, Q, N, H |
| 556 | E | R, G, A, K, Q, N, H |
| 557 | N | R, G, A, K, Q, H |
| 558 | R | G, A, K, Q, N, H |
| 559 | S | R, G, A, K, Q, N, H |
| 560 | K | R, G, A, Q, N, H |
| 561 | R | G, A, K, Q, N, H |
| 562 | F | R, G, A, K, Q, N, H |
| 563 | G | R, A, K, Q, N, H |
| 564 | Y | R, G, A, K, Q, N, H |
| 565 | R | G, A, K, Q, N, H |
| 566 | H | R, G, A, K, Q, N |
| 567 | S | R, G, A, K, Q, N, H |
| 568 | T | R, G, A, K, Q, N, H |
| 569 | N | R, G, A, K, Q, H |
| 570 | L | R, G, A, K, Q, N, H |
| 571 | S | R, G, A, K, Q, N, H |
| 572 | N | R, G, A, K, Q, H |
| 573 | E | R, G, A, K, Q, N, H |
| 574 | S | R, G, A, K, Q, N, H |
| 575 | R | G, A, K, Q, N, H |
| 576 | Q | R, G, A, K, N, H |
| 577 | I | R, G, A, K, Q, N, H |
| 578 | L | R, G, A, K, Q, N, H |
| 579 | L | R, G, A, K, Q, N, H |
| 580 | D | R, G, A, K, Q, N, H |
| 581 | K | R, G, A, Q, N, H |
| 582 | S | R, G, A, K, Q, N, H |
| 583 | K | R, G, A, Q, N, H |
| 584 | Y | R, G, A, K, Q, N, H |
| 585 | A | R, G, K, Q, N, H |
| 586 | K | R, G, A, Q, N, H |
| 587 | A | R, G, K, Q, N, H |
| 588 | N | R, G, A, K, Q, H |
| 589 | K | R, G, A, Q, N, H |
| 590 | S | R, G, A, K, Q, N, H |
| 591 | V | R, G, A, K, Q, N, H |
| 592 | L | R, G, A, K, Q, N, H |
| 593 | R | G, A, K, Q, N, H |
| 594 | C | R, G, A, K, Q, N, H |
| 595 | M | R, G, A, K, Q, N, H |
| 596 | E | R, G, A, K, Q, N, H |
| 597 | N | R, G, A, K, Q, H |
| 598 | M | R, G, A, K, Q, N, H |
| 599 | T | R, G, A, K, Q, N, H |
| 600 | H | R, G, A, K, Q, N |
| 601 | N | R, G, A, K, Q, H |
| 602 | V | R, G, A, K, Q, N, H |
| 603 | V | R, G, A, K, Q, N, H |
| 604 | F | R, G, A, K, Q, N, H |
| 605 | D | R, G, A, K, Q, N, H |
| 606 | P | R, G, A, K, Q, N, H |
| 607 | K | R, G, A, Q, N, H |
| 608 | T | R, G, A, K, Q, N, H |
| 609 | S | R, G, A, K, Q, N, H |
| 610 | L | R, G, A, K, Q, N, H |
| 611 | N | R, G, A, K, Q, H |
| 612 | I | R, G, A, K, Q, N, H |
| 613 | R | G, A, K, Q, N, H |
| 614 | I | R, G, A, K, Q, N, H |
| 615 | K | R, G, A, Q, N, H |
| 616 | T | R, G, A, K, Q, N, H |
| 617 | D | R, G, A, K, Q, N, H |
| 618 | K | R, G, A, Q, N, H |
| 619 | N | R, G, A, K, Q, H |
| 620 | N | R, G, A, K, Q, H |
| 621 | S | R, G, A, K, Q, N, H |
| 622 | P | R, G, A, K, Q, N, H |
| 623 | V | R, G, A, K, Q, N, H |
| 624 | L | R, G, A, K, Q, N, H |
| 625 | D | R, G, A, K, Q, N, H |
| 626 | D | R, G, A, K, Q, N, H |
| 627 | K | R, G, A, Q, N, H |
| 628 | G | R, A, K, Q, N, H |
| 629 | R | G, A, K, Q, N, H |
| 630 | I | R, G, A, K, Q, N, H |
| 631 | T | R, G, A, K, Q, N, H |
| 632 | F | R, G, A, K, Q, N, H |
| 633 | V | R, G, A, K, Q, N, H |
| 634 | M | R, G, A, K, Q, N, H |
| 635 | Q | R, G, A, K, N, H |
| 636 | I | R, G, A, K, Q, N, H |
| 637 | N | R, G, A, K, Q, H |
| 638 | H | R, G, A, K, Q, N |
| 639 | R | G, A, K, Q, N, H |
| 640 | I | R, G, A, K, Q, N, H |
| 641 | L | R, G, A, K, Q, N, H |
| 642 | E | R, G, A, K, Q, N, H |
| 643 | K | R, G, A, Q, N, H |
| 644 | Y | R, G, A, K, Q, N, H |
| 645 | N | R, G, A, K, Q, H |
| 646 | N | R, G, A, K, Q, H |
| 647 | T | R, G, A, K, Q, N, H |
| 648 | K | R, G, A, Q, N, H |
| 649 | I | R, G, A, K, Q, N, H |
| 650 | E | R, G, A, K, Q, N, H |
| 651 | I | R, G, A, K, Q, N, H |
| 652 | G | R, A, K, Q, N, H |
| 653 | D | R, G, A, K, Q, N, H |
| 654 | R | G, A, K, Q, N, H |
| 655 | I | R, G, A, K, Q, N, H |
| 656 | L | R, G, A, K, Q, N, H |
| 657 | A | R, G, K, Q, N, H |
| 658 | Y | R, G, A, K, Q, N, H |
| 659 | D | A |
| 660 | Q | R, G, A, K, N, H |
| 661 | N | R, G, A, K, Q, H |
| 662 | Q | R, G, A, K, N, H |
| 663 | S | R, G, A, K, Q, N, H |
| 664 | E | R, G, A, K, Q, N, H |
| 665 | N | R, G, A, K, Q, H |
| 666 | H | R, G, A, K, Q, N |
| 667 | T | R, G, A, K, Q, N, H |
| 668 | Y | R, G, A, K, Q, N, H |
| 669 | A | R, G, K, Q, N, H |
| 670 | I | R, G, A, K, Q, N, H |
| 671 | L | R, G, A, K, Q, N, H |
| 672 | Q | R, G, A, K, N, H |
| 673 | R | G, A, K, Q, N, H |
| 674 | T | R, G, A, K, Q, N, H |
| 675 | E | R, G, A, K, Q, N, H |
| 676 | E | R, G, A, K, Q, N, H |
| 677 | G | R, A, K, Q, N, H |
| 678 | S | R, G, A, K, Q, N, H |
| 679 | H | R, G, A, K, Q, N |
| 680 | A | R, G, K, Q, N, H |
| 681 | H | R, G, A, K, Q, N |
| 682 | Q | R, G, A, K, N, H |
| 683 | F | R, G, A, K, Q, N, H |
| 684 | N | R, G, A, K, Q, H |
| 685 | G | R, A, K, Q, N, H |
| 686 | W | R, G, A, K, Q, N, H |
| 687 | Y | R, G, A, K, Q, N, H |
| 688 | V | R, G, A, K, Q, N, H |
| 689 | R | G, A, K, Q, N, H |
| 690 | V | R, G, A, K, Q, N, H |
| 691 | L | R, G, A, K, Q, N, H |
| 692 | E | R, G, A, K, Q, N, H |
| 693 | T | R, G, A, K, Q, N, H |
| 694 | G | R, A, K, Q, N, H |
| 695 | K | R, G, A, Q, N, H |
| 696 | V | R, G, A, K, Q, N, H |
| 697 | T | R, G, A, K, Q, N, H |
| 698 | S | R, G, A, K, Q, N, H |
| 699 | I | R, G, A, K, Q, N, H |
| 700 | V | R, G, A, K, Q, N, H |
| 701 | Q | R, G, A, K, N, H |
| 702 | G | R, A, K, Q, N, H |
| 703 | L | R, G, A, K, Q, N, H |
| 704 | S | R, G, A, K, Q, N, H |
| 705 | G | R, A, K, Q, N, H |
| 706 | P | R, G, A, K, Q, N, H |
| 707 | I | R, G, A, K, Q, N, H |
| 708 | D | R, G, A, K, Q, N, H |
| 709 | Q | R, G, A, K, N, H |
| 710 | L | R, G, A, K, Q, N, H |
| 711 | N | R, G, A, K, Q, H |
| 712 | Y | R, G, A, K, Q, N, H |
| 713 | D | R, G, A, K, Q, N, H |
| 714 | G | R, A, K, Q, N, H |
| 715 | M | R, G, A, K, Q, N, H |
| 716 | P | R, G, A, K, Q, N, H |
| 717 | V | R, G, A, K, Q, N, H |
| 718 | T | R, G, A, K, Q, N, H |
| 719 | S | R, G, A, K, Q, N, H |
| 720 | H | R, G, A, K, Q, N |
| 721 | K | R, G, A, Q, N, H |
| 722 | F | R, G, A, K, Q, N, H |
| 723 | N | R, G, A, K, Q, H |
| 724 | C | R, G, A, K, Q, N, H |
| 725 | W | R, G, A, K, Q, N, H |
| 726 | Q | R, G, A, K, N, H |
| 727 | A | R, G, K, Q, N, H |
| 728 | D | R, G, A, K, Q, N, H |
| 729 | R | G, A, K, Q, N, H |
| 730 | S | R, G, A, K, Q, N, H |
| 731 | A | R, G, K, Q, N, H |
| 732 | F | R, G, A, K, Q, N, H |
| 733 | V | R, G, A, K, Q, N, H |
| 734 | S | R, G, A, K, Q, N, H |
| 735 | Q | R, G, A, K, N, H |
| 736 | F | R, G, A, K, Q, N, H |
| 737 | A | R, G, K, Q, N, H |
| 738 | S | R, G, A, K, Q, N, H |
| 739 | L | R, G, A, K, Q, N, H |
| 740 | K | R, G, A, Q, N, H |
| 741 | I | R, G, A, K, Q, N, H |
| 742 | S | R, G, A, K, Q, N, H |
| 743 | E | R, G, A, K, Q, N, H |
| 744 | T | R, G, A, K, Q, N, H |
| 745 | E | R, G, A, K, Q, N, H |
| 746 | T | R, G, A, K, Q, N, H |
| 747 | F | R, G, A, K, Q, N, H |
| 748 | D | R, G, A, K, Q, N, H |
| 749 | E | R, G, A, K, Q, N, H |
| 750 | A | R, G, K, Q, N, H |
| 751 | Y | R, G, A, K, Q, N, H |
| 752 | Q | R, G, A, K, N, H |
| 753 | A | R, G, K, Q, N, H |
| 754 | I | R, G, A, K, Q, N, H |
| 755 | N | R, G, A, K, Q, H |
| 756 | A | R, G, K, Q, N, H |
| 757 | Q | R, G, A, K, N, H |
| 758 | G | R, A, K, Q, N, H |
| 759 | A | R, G, K, Q, N, H |
| 760 | Y | R, G, A, K, Q, N, H |
| 761 | T | R, G, A, K, Q, N, H |
| 762 | W | R, G, A, K, Q, N, H |
| 763 | N | R, G, A, K, Q, H |
| 764 | L | R, G, A, K, Q, N, H |
| 765 | F | R, G, A, K, Q, N, H |
| 766 | Y | R, G, A, K, Q, N, H |
| 767 | L | R, G, A, K, Q, N, H |
| 768 | R | G, A, K, Q, N, H |
| 769 | I | R, G, A, K, Q, N, H |
| 770 | L | R, G, A, K, Q, N, H |
| 771 | R | G, A, K, Q, N, H |
| 772 | K | R, G, A, Q, N, H |
| 773 | A | R, G, K, Q, N, H |
| 774 | L | R, G, A, K, Q, N, H |
| 775 | R | G, A, K, Q, N, H |
| 776 | V | R, G, A, K, Q, N, H |
| 777 | C | R, G, A, K, Q, N, H |
| 778 | H | R, G, A, K, Q, N |
| 779 | M | R, G, A, K, Q, N, H |
| 780 | E | R, G, A, K, Q, N, H |
| 781 | N | R, G, A, K, Q, H |
| 782 | I | R, G, A, K, Q, N, H |
| 783 | N | R, G, A, K, Q, H |
| 784 | Q | R, G, A, K, N, H |
| 785 | F | R, G, A, K, Q, N, H |
| 786 | R | G, A, K, Q, N, H |
| 787 | E | R, G, A, K, Q, N, H |
| 788 | E | R, G, A, K, Q, N, H |
| 789 | I | R, G, A, K, Q, N, H |
| 790 | L | R, G, A, K, Q, N, H |
| 791 | A | R, G, K, Q, N, H |
| 792 | I | R, G, A, K, Q, N, H |
| 793 | S | R, G, A, K, Q, N, H |
| 794 | K | R, G, A, Q, N, H |
| 795 | N | R, G, A, K, Q, H |
| 796 | R | G, A, K, Q, N, H |
| 797 | L | R, G, A, K, Q, N, H |
| 798 | S | R, G, A, K, Q, N, H |
| 799 | P | R, G, A, K, Q, N, H |
| 800 | M | R, G, A, K, Q, N, H |
| 801 | S | R, G, A, K, Q, N, H |
| 802 | L | R, G, A, K, Q, N, H |
| 803 | G | R, A, K, Q, N, H |
| 804 | S | R, G, A, K, Q, N, H |
| 805 | L | R, G, A, K, Q, N, H |
| 806 | S | R, G, A, K, Q, N, H |
| 807 | Q | R, G, A, K, N, H |
| 808 | N | R, G, A, K, Q, H |
| 809 | S | R, G, A, K, Q, N, H |
| 810 | L | R, G, A, K, Q, N, H |
| 811 | K | R, G, A, Q, N, H |
| 812 | M | R, G, A, K, Q, N, H |
| 813 | I | R, G, A, K, Q, N, H |
| 814 | R | G, A, K, Q, N, H |
| 815 | A | R, G, K, Q, N, H |
| 816 | F | R, G, A, K, Q, N, H |
| 817 | K | R, G, A, Q, N, H |
| 818 | S | R, G, A, K, Q, N, H |
| 819 | I | R, G, A, K, Q, N, H |
| 820 | I | R, G, A, K, Q, N, H |
| 821 | N | R, G, A, K, Q, H |
| 822 | C | R, G, A, K, Q, N, H |
| 823 | Y | R, G, A, K, Q, N, H |
| 824 | M | R, G, A, K, Q, N, H |
| 825 | S | R, G, A, K, Q, N, H |
| 826 | R | G, A, K, Q, N, H |
| 827 | M | R, G, A, K, Q, N, H |
| 828 | S | R, G, A, K, Q, N, H |
| 829 | F | R, G, A, K, Q, N, H |
| 830 | V | R, G, A, K, Q, N, H |
| 831 | D | R, G, A, K, Q, N, H |
| 832 | E | R, G, A, K, Q, N, H |
| 833 | L | R, G, A, K, Q, N, H |
| 834 | Q | R, G, A, K, N, H |
| 835 | K | R, G, A, Q, N, H |
| 836 | K | R, G, A, Q, N, H |
| 837 | E | R, G, A, K, Q, N, H |
| 838 | G | R, A, K, Q, N, H |
| 839 | D | R, G, A, K, Q, N, H |
| 840 | L | R, G, A, K, Q, N, H |
| 841 | E | R, G, A, K, Q, N, H |
| 842 | L | R, G, A, K, Q, N, H |
| 843 | H | R, G, A, K, Q, N |
| 844 | T | R, G, A, K, Q, N, H |
| 845 | I | R, G, A, K, Q, N, H |
| 846 | M | R, G, A, K, Q, N, H |
| 847 | R | G, A, K, Q, N, H |
| 848 | L | R, G, A, K, Q, N, H |
| 849 | T | R, G, A, K, Q, N, H |
| 850 | D | R, G, A, K, Q, N, H |
| 851 | N | R, G, A, K, Q, H |
| 852 | K | R, G, A, Q, N, H |
| 853 | L | R, G, A, K, Q, N, H |
| 854 | N | R, G, A, K, Q, H |
| 855 | D | R, G, A, K, Q, N, H |
| 856 | K | R, G, A, Q, N, H |
| 857 | R | G, A, K, Q, N, H |
| 858 | V | R, G, A, K, Q, N, H |
| 859 | E | R, G, A, K, Q, N, H |
| 860 | K | R, G, A, Q, N, H |
| 861 | I | R, G, A, K, Q, N, H |
| 862 | N | R, G, A, K, Q, H |
| 863 | R | G, A, K, Q, N, H |
| 864 | A | R, G, K, Q, N, H |
| 865 | S | R, G, A, K, Q, N, H |
| 866 | S | R, G, A, K, Q, N, H |
| 867 | F | R, G, A, K, Q, N, H |
| 868 | L | R, G, A, K, Q, N, H |
| 869 | T | R, G, A, K, Q, N, H |
| 870 | N | R, G, A, K, Q, H |
| 871 | K | R, G, A, Q, N, H |
| 872 | A | R, G, K, Q, N, H |
| 873 | H | R, G, A, K, Q, N |
| 874 | S | R, G, A, K, Q, N, H |
| 875 | M | R, G, A, K, Q, N, H |
| 876 | G | R, A, K, Q, N, H |
| 877 | C | R, G, A, K, Q, N, H |
| 878 | K | R, G, A, Q, N, H |
| 879 | M | R, G, A, K, Q, N, H |
| 880 | I | R, G, A, K, Q, N, H |
| 881 | V | R, G, A, K, Q, N, H |
| 882 | G | R, A, K, Q, N, H |
| 883 | E | A |
| 884 | S | R, G, A, K, Q, N, H |
| 885 | D | R, G, A, K, Q, N, H |
| 886 | L | R, G, A, K, Q, N, H |
| 887 | P | R, G, A, K, Q, N, H |
| 888 | V | R, G, A, K, Q, N, H |
| 889 | A | R, G, K, Q, N, H |
| 890 | D | R, G, A, K, Q, N, H |
| 891 | S | R, G, A, K, Q, N, H |
| 892 | K | R, G, A, Q, N, H |
| 893 | T | R, G, A, K, Q, N, H |
| 894 | S | R, G, A, K, Q, N, H |
| 895 | K | R, G, A, Q, N, H |
| 896 | K | R, G, A, Q, N, H |
| 897 | Q | R, G, A, K, N, H |
| 898 | N | R, G, A, K, Q, H |
| 899 | V | R, G, A, K, Q, N, H |
| 900 | D | R, G, A, K, Q, N, H |
| 901 | R | G, A, K, Q, N, H |
| 902 | M | R, G, A, K, Q, N, H |
| 903 | D | R, G, A, K, Q, N, H |
| 904 | W | R, G, A, K, Q, N, H |
| 905 | C | R, G, A, K, Q, N, H |
| 906 | A | R, G, K, Q, N, H |
| 907 | R | G, A, K, Q, N, H |
| 908 | A | R, G, K, Q, N, H |
| 909 | L | R, G, A, K, Q, N, H |
| 910 | S | R, G, A, K, Q, N, H |
| 911 | H | R, G, A, K, Q, N |
| 912 | K | R, G, A, Q, N, H |
| 913 | V | R, G, A, K, Q, N, H |
| 914 | E | R, G, A, K, Q, N, H |
| 915 | Y | R, G, A, K, Q, N, H |
| 916 | A | R, G, K, Q, N, H |
| 917 | C | R, G, A, K, Q, N, H |
| 918 | K | R, G, A, Q, N, H |
| 919 | L | R, G, A, K, Q, N, H |
| 920 | M | R, G, A, K, Q, N, H |
| 921 | G | R, A, K, Q, N, H |
| 922 | L | R, G, A, K, Q, N, H |
| 923 | A | R, G, K, Q, N, H |
| 924 | Y | R, G, A, K, Q, N, H |
| 925 | R | G, A, K, Q, N, H |
| 926 | G | R, A, K, Q, N, H |
| 927 | I | R, G, A, K, Q, N, H |
| 928 | P | R, G, A, K, Q, N, H |
| 929 | A | R, G, K, Q, N, H |
| 930 | Y | R, G, A, K, Q, N, H |
| 931 | M | R, G, A, K, Q, N, H |
| 932 | S | R, G, A, K, Q, N, H |
| 933 | S | R, G, A, K, Q, N, H |
| 934 | H | R, G, A, K, Q, N |
| 935 | Q | R, G, A, K, N, H |
| 936 | D | R, G, A, K, Q, N, H |
| 937 | P | R, G, A, K, Q, N, H |
| 938 | L | R, G, A, K, Q, N, H |
| 939 | V | R, G, A, K, Q, N, H |
| 940 | H | R, G, A, K, Q, N |
| 941 | L | R, G, A, K, Q, N, H |
| 942 | V | R, G, A, K, Q, N, H |
| 943 | E | R, G, A, K, Q, N, H |
| 944 | S | R, G, A, K, Q, N, H |
| 945 | K | R, G, A, Q, N, H |
| 946 | R | G, A, K, Q, N, H |
| 947 | S | R, G, A, K, Q, N, H |
| 948 | V | R, G, A, K, Q, N, H |
| 949 | L | R, G, A, K, Q, N, H |
| 950 | R | G, A, K, Q, N, H |
| 951 | P | R, G, A, K, Q, N, H |
| 952 | R | G, A, K, Q, N, H |
| 953 | F | R, G, A, K, Q, N, H |
| 954 | V | R, G, A, K, Q, N, H |
| 955 | V | R, G, A, K, Q, N, H |
| 956 | A | R, G, K, Q, N, H |
| 957 | D | R, G, A, K, Q, N, H |
| 958 | K | R, G, A, Q, N, H |
| 959 | S | R, G, A, K, Q, N, H |
| 960 | D | R, G, A, K, Q, N, H |
| 961 | V | R, G, A, K, Q, N, H |
| 962 | K | R, G, A, Q, N, H |
| 963 | Q | R, G, A, K, N, H |
| 964 | H | R, G, A, K, Q, N |
| 965 | H | R, G, A, K, Q, N |
| 966 | L | R, G, A, K, Q, N, H |
| 967 | D | R, G, A, K, Q, N, H |
| 968 | N | R, G, A, K, Q, H |
| 969 | L | R, G, A, K, Q, N, H |
| 970 | R | G, A, K, Q, N, H |
| 971 | R | G, A, K, Q, N, H |
| 972 | M | R, G, A, K, Q, N, H |
| 973 | L | R, G, A, K, Q, N, H |
| 974 | N | R, G, A, K, Q, H |
| 975 | S | R, G, A, K, Q, N, H |
| 976 | K | R, G, A, Q, N, H |
| 977 | T | R, G, A, K, Q, N, H |
| 978 | K | R, G, A, Q, N, H |
| 979 | V | R, G, A, K, Q, N, H |
| 980 | G | R, A, K, Q, N, H |
| 981 | T | R, G, A, K, Q, N, H |
| 982 | A | R, G, K, Q, N, H |
| 983 | V | R, G, A, K, Q, N, H |
| 984 | Y | R, G, A, K, Q, N, H |
| 985 | Y | R, G, A, K, Q, N, H |
| 986 | R | G, A, K, Q, N, H |
| 987 | E | R, G, A, K, Q, N, H |
| 988 | A | R, G, K, Q, N, H |
| 989 | V | R, G, A, K, Q, N, H |
| 990 | E | R, G, A, K, Q, N, H |
| 991 | L | R, G, A, K, Q, N, H |
| 992 | M | R, G, A, K, Q, N, H |
| 993 | C | R, G, A, K, Q, N, H |
| 994 | E | R, G, A, K, Q, N, H |
| 995 | E | R, G, A, K, Q, N, H |
| 996 | L | R, G, A, K, Q, N, H |
| 997 | G | R, A, K, Q, N, H |
| 998 | I | R, G, A, K, Q, N, H |
| 999 | H | R, G, A, K, Q, N |
| 1000 | K | R, G, A, Q, N, H |
| 1001 | T | R, G, A, K, Q, N, H |
| 1002 | D | R, G, A, K, Q, N, H |
| 1003 | M | R, G, A, K, Q, N, H |
| 1004 | A | R, G, K, Q, N, H |
| 1005 | K | R, G, A, Q, N, H |
| 1006 | G | R, A, K, Q, N, H |
| 1007 | K | R, G, A, Q, N, H |
| 1008 | V | R, G, A, K, Q, N, H |
| 1009 | S | R, G, A, K, Q, N, H |
| 1010 | L | R, G, A, K, Q, N, H |
| 1011 | S | R, G, A, K, Q, N, H |
| 1012 | D | R, G, A, K, Q, N, H |
| 1013 | F | R, G, A, K, Q, N, H |
| 1014 | V | R, G, A, K, Q, N, H |
| 1015 | D | R, G, A, K, Q, N, H |
| 1016 | K | R, G, A, Q, N, H |
| 1017 | F | R, G, A, K, Q, N, H |
| 1018 | I | R, G, A, K, Q, N, H |
| 1019 | G | R, A, K, Q, N, H |
| 1020 | E | R, G, A, K, Q, N, H |
| 1021 | K | R, G, A, Q, N, H |
| 1022 | A | R, G, K, Q, N, H |
| 1023 | I | R, G, A, K, Q, N, H |
| 1024 | F | R, G, A, K, Q, N, H |
| 1025 | P | R, G, A, K, Q, N, H |
| 1026 | Q | R, G, A, K, N, H |
| 1027 | R | G, A, K, Q, N, H |
| 1028 | G | R, A, K, Q, N, H |
| 1029 | G | R, A, K, Q, N, H |
| 1030 | R | G, A, K, Q, N, H |
| 1031 | F | R, G, A, K, Q, N, H |
| 1032 | Y | R, G, A, K, Q, N, H |
| 1033 | M | R, G, A, K, Q, N, H |
| 1034 | S | R, G, A, K, Q, N, H |
| 1035 | T | R, G, A, K, Q, N, H |
| 1036 | K | R, G, A, Q, N, H |
| 1037 | R | G, A, K, Q, N, H |
| 1038 | L | R, G, A, K, Q, N, H |
| 1039 | T | R, G, A, K, Q, N, H |
| 1040 | T | R, G, A, K, Q, N, H |
| 1041 | G | R, A, K, Q, N, H |
| 1042 | A | R, G, K, Q, N, H |
| 1043 | K | R, G, A, Q, N, H |
| 1044 | L | R, G, A, K, Q, N, H |
| 1045 | I | R, G, A, K, Q, N, H |
| 1046 | C | R, G, A, K, Q, N, H |
| 1047 | Y | R, G, A, K, Q, N, H |
| 1048 | S | R, G, A, K, Q, N, H |
| 1049 | G | R, A, K, Q, N, H |
| 1050 | S | R, G, A, K, Q, N, H |
| 1051 | D | R, G, A, K, Q, N, H |
| 1052 | V | R, G, A, K, Q, N, H |
| 1053 | W | R, G, A, K, Q, N, H |
| 1054 | L | R, G, A, K, Q, N, H |
| 1055 | S | R, G, A, K, Q, N, H |
| 1056 | D | R, G, A, K, Q, N, H |
| 1057 | A | R, G, K, Q, N, H |
| 1058 | D | R, G, A, K, Q, N, H |
| 1059 | E | R, G, A, K, Q, N, H |
| 1060 | I | R, G, A, K, Q, N, H |
| 1061 | A | R, G, K, Q, N, H |
| 1062 | A | R, G, K, Q, N, H |
| 1063 | I | R, G, A, K, Q, N, H |
| 1064 | N | R, G, A, K, Q, H |
| 1065 | I | R, G, A, K, Q, N, H |
| 1066 | G | R, A, K, Q, N, H |
| 1067 | M | R, G, A, K, Q, N, H |
| 1068 | F | R, G, A, K, Q, N, H |
| 1069 | V | R, G, A, K, Q, N, H |
| 1070 | V | R, G, A, K, Q, N, H |
| 1071 | C | R, G, A, K, Q, N, H |
| 1072 | D | R, G, A, K, Q, N, H |
| 1073 | Q | R, G, A, K, N, H |
| 1074 | T | R, G, A, K, Q, N, H |
| 1075 | G | R, A, K, Q, N, H |
| 1076 | A | R, G, K, Q, N, H |
| 1077 | F | R, G, A, K, Q, N, H |
| 1078 | K | R, G, A, Q, N, H |
| 1079 | K | R, G, A, Q, N, H |
| 1080 | K | R, G, A, Q, N, H |
| 1081 | K | R, G, A, Q, N, H |
| 1082 | K | R, G, A, Q, N, H |
| 1083 | E | R, G, A, K, Q, N, H |
| 1084 | K | R, G, A, Q, N, H |
| 1085 | L | R, G, A, K, Q, N, H |
| 1086 | D | R, G, A, K, Q, N, H |
| 1087 | D | R, G, A, K, Q, N, H |
| 1088 | E | R, G, A, K, Q, N, H |
| 1089 | E | R, G, A, K, Q, N, H |
| 1090 | C | R, G, A, K, Q, N, H |
| 1091 | D | R, G, A, K, Q, N, H |
| 1092 | I | R, G, A, K, Q, N, H |
| 1093 | L | R, G, A, K, Q, N, H |
| 1094 | P | R, G, A, K, Q, N, H |
| 1095 | F | R, G, A, K, Q, N, H |
| 1096 | R | G, A, K, Q, N, H |
| 1097 | P | R, G, A, K, Q, N, H |
| 1098 | M | R, G, A, K, Q, N, H |

In some embodiments, the variant Cas12i3 polypeptide comprises an alteration that increases interactions of the variant Cas12i3 polypeptide to the RNA guide. In some embodiments, the alteration that increases interactions with the RNA guide is an arginine, lysine, glutamine, asparagine, or histidine substitution. In some embodiments, the variant Cas12i3 polypeptide comprises an alteration that increases interactions of the variant Cas12i3 polypeptide to the target nucleic acid. In some embodiments, the alteration that increases interactions with the target nucleic acid is an arginine, lysine, glutamine, asparagine, or histidine substitution. In some embodiments, the variant Cas12i3 polypeptide comprises an alanine substitution. In some embodiments, the alanine substitution does not affect the geometry of the variant Cas12i3 polypeptide backbone. In some embodiments, the variant Cas12i3 polypeptide comprises a glycine substitution. In some embodiments, the glycine substitution alters the Ramachandran bond angles of the variant Cas12i3 polypeptide backbone.

In some embodiments, the variant Cas12i3 polypeptide comprises at least one RuvC motif or a RuvC domain. As used herein, a “biologically active portion” is a portion that retains at least one function (e.g. completely, partially, minimally) of the parent polypeptide (e.g., a “minimal” or “core” domain). In some embodiments, the variant Cas12i3 polypeptide retains enzymatic activity at least as active as the parent polypeptide. Accordingly, in some embodiments, a variant Cas12i3 polypeptide has enzymatic activity greater than the parent polypeptide.

In some embodiments, the variant Cas12i3 polypeptide has reduced nuclease activity or is a nuclease dead polypeptide. As used herein, the catalytic residues of a polypeptide disclosed herein are D659, E883, and D936 or D1058. In some embodiments, a variant Cas12i3 polypeptide comprising a substitution at one or more of D395, E883, and D936 or D1058 (e.g., D395A, E883A, and D936A or D1058A) exhibits reduced nuclease activity or no nuclease activity relative to a parent polypeptide.

In some embodiments, the variant Cas12i3 polypeptide comprises a substitution of Table 3. In some embodiments, the variant Cas12i3 polypeptide comprises one or more substitutions of Table 3. In some embodiments, the variant Cas12i3 polypeptide comprises one or more of the substitutions of Table 4. In some embodiments, the variant Cas12i3 polypeptide comprises one or more substitutions of Table 6.

TABLE 3
Single Cas12i3 Amino Acid Substitutions.
Substitutions

H566R
S571R
E596R
S738R
S536R
S559R
L592R
N646R
K583R
K586R
M595R
S704R
N711R
C724R
T844R
T977R
T1040R
R565G
I630R
L703R
S801R
T631R

TABLE 4
Combination Cas12i3 Amino Acid Substitutions.
Substitutions

S536R N711R S801R
S536R N711R C724R S801R
S536R N711R C724R
S536R N711R
S536R K583R N711R S801R
S536R K583R N711R C724R
S536R K583R C724R S801R
S536R K583R C724R
S536R K583R
S536R C724R S801R
S536R C724R
N711R S801R
N711R C724R S801R
N711R C724R
K583R S801R
K583R N711R S801R
K583R N711R C724R S801R
K583R N711R C724R
K583R N711R
K583R C724R S801R
K583R C724R
C724R S801R

In some embodiments, the variant Cas12i3 polypeptide comprises at least 70% (70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) identity to SEQ ID NO: 7. In some embodiments, the variant Cas12i3 polypeptide comprises at least 70% (70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or greater) identity to SEQ ID NO: 7 and further comprises a substitution of any one of Tables 2-4 or 6. In some embodiments, the variant Cas12i3 polypeptide comprises at least 90% (90%, 91%, 92%, 93%, 94%, 95%, 96%, 97%, 98%, 99%, or greater) identity to SEQ ID NO: 7 and further comprises a substitution of any one of Tables 2-4 or 6. In some embodiments, the variant Cas12i3 polypeptide comprises at least 95% (95%, 96%, 97%, 98%, 99%, or greater) identity to SEQ ID NO: 7 and further comprises a substitution of any one of Tables 2-4 or 6. In some embodiments, the variant Cas12i3 polypeptide comprises one or more of the following substitutions: K583R N711R C724R and further comprises one or more substitutions listed in any one of Tables 2-4 or 6.

In some embodiments, a variant Cas12i3 polypeptide comprises an insertion relative to the sequence of SEQ ID NO: 3 or SEQ ID NO: 7. In some embodiments, the insertion comprises one residue to about 10 residues in length (e.g., 1, 2, 3, 4, 5, 6, 7, 8, 9, or 10 residues). In some embodiments, the insertion comprises one or more of a glycine, serine, aspartate, or asparagine residue. In some embodiments, the insertion comprises a one-residue insertion (e.g., one glycine, one serine, one aspartate, or one asparagine). In some embodiments, the insertion comprises a two-residue insertion (e.g., two glycines, two serines, two aspartates, or two asparagines). In some embodiments, the insertion comprises a two-residue insertion comprising at least one glycine. In some embodiments, the insertion comprises a three-residue insertion (e.g., three glycines, three serines, three aspartates, or three asparagines). In some embodiments, the insertion comprises a three-residue insertion comprising at least one glycine. In some embodiments, the insertion comprises a four-residue insertion (e.g., four glycines, four serines, four aspartates, or four asparagines). In some embodiments, the insertion comprises a four-residue insertion comprising at least one glycine. In some embodiments, the insertion comprises a five-residue insertion (e.g., five glycines, five serines, five aspartates, or five asparagines). In some embodiments, the insertion comprises a five-residue insertion comprising at least one glycine. In some embodiments, the insertion occurs within the Wed domain, the Rec1 domain, or the Nuc domain. In some embodiments, the insertion occurs at the interface of the Wed domain and the Rec1 domain.

In some embodiments, a variant Cas12i3 polypeptide has a glycine-glycine, serine-serine, aspartate-aspartate, asparagine-asparagine, glycine-serine, glycine-aspartate, glycine-asparagine, serine-glycine, aspartate-glycine, or asparagine-glycine insertion. In some embodiments, the glycine-glycine, serine-serine, aspartate-aspartate, asparagine-asparagine, glycine-serine, glycine-aspartate, glycine-asparagine, serine-glycine, aspartate-glycine, or asparagine-glycine insertion is in the Wed domain, the Rec1 domain, and/or the Nuc domain.

In some embodiments, the variant Cas12i3 polypeptide of the present invention has enzymatic activity equivalent to or greater than the parent polypeptide. In some embodiments, the variant Cas12i3 polypeptide of the present invention has enzymatic activity at a temperature range from about 20° C. to about 90° C. In some embodiments, the variant Cas12i3 polypeptide of the present invention has enzymatic activity at a temperature of about 20° C. to about 25° C. or at a temperature of about 37° C.

In some embodiments, the variant Cas12i3 polypeptide comprises at least one alteration that enhances affinity to RNA (e.g., RNA affinity), as compared to a parent polypeptide. In some embodiments, the variant Cas12i3 polypeptide exhibits enhanced RNA affinity, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant Cas12i3 polypeptide exhibits enhanced RNA affinity, as compared to a parent polypeptide, in a buffer having a pH in a range of about 7.3 to about 8.6. In some embodiments, the variant Cas12i3 polypeptide exhibits enhanced RNA affinity, as compared to a parent polypeptide, when the Tₘ value of the variant Cas12i3 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant Cas12i3 polypeptide exhibits enhanced RNA affinity when the Tₘ value of the variant Cas12i3 polypeptide is at least 8° C. greater than the Tₘ value of the parent polypeptide.

In some embodiments, at least one alteration is introduced into the parent polypeptide of SEQ ID NO: 3 to produce a variant Cas12i3 polypeptide that exhibits (a) decreased enzymatic activity and (b) enhanced RNA affinity relative to the parent polypeptide of SEQ ID NO: 3. In some embodiments, at least one alteration is introduced into the parent polypeptide of SEQ ID NO: 3 to produce a variant Cas12i3 polypeptide that exhibits (a) increased enzymatic activity and (b) enhanced RNA affinity, relative to the parent polypeptide of SEQ ID NO: 3. In some embodiments, at least one alteration is introduced into the parent polypeptide of SEQ ID NO: 3 to produce a variant Cas12i3 polypeptide that exhibits (a) retained enzymatic activity and (b) enhanced RNA affinity, relative to the parent polypeptide of SEQ ID NO: 3.

In some embodiments, the variant Cas12i3 polypeptide comprises at least one alteration that enhances complex formation with an RNA guide (e.g., binary complex formation), as compared to a parent polypeptide. In some embodiments, the variant Cas12i3 polypeptide exhibits enhanced binary complex formation, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant Cas12i3 polypeptide exhibits enhanced binary complex formation, as compared to a parent polypeptide, in a buffer having a pH in a range of about 7.3 to about 8.6. In some embodiments, the variant Cas12i3 polypeptide exhibits enhanced binary complex formation, as compared to a parent polypeptide, when the Tₘ value of the variant Cas12i3 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C. or 20° C. greater than the Tₘ value of a parent polypeptide. In one embodiment, the variant Cas12i3 polypeptide exhibits enhanced binary complex formation when the Tₘ value of the variant Cas12i3 polypeptide is at least 8° C. greater than the Tₘ value of the parent polypeptide.

In some embodiments, at least one alteration is introduced into the parent polypeptide of SEQ ID NO: 3 to produce a variant Cas12i3 polypeptide that exhibits (a) decreased enzymatic activity and (b) enhanced binary complex formation relative to the parent polypeptide of SEQ ID NO: 3. In some embodiments, at least one alteration is introduced into the parent polypeptide of SEQ ID NO: 3 to produce a variant Cas12i3 polypeptide that exhibits (a) increased enzymatic activity and (b) enhanced binary complex formation, relative to the parent polypeptide of SEQ ID NO: 3. In some embodiments, at least one alteration is introduced into the parent polypeptide of SEQ ID NO: 3 to produce a variant Cas12i3 polypeptide that exhibits (a) retained enzymatic activity and (b) enhanced binary complex formation, relative to the parent polypeptide of SEQ ID NO: 3.

In some embodiments, the variant Cas12i3 polypeptide comprises at least one alteration that enhances binding activity to an RNA guide, as compared to a parent polypeptide. In some embodiments, the variant Cas12i3 polypeptide exhibits enhanced RNA guide binding activity, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant Cas12i3 polypeptide exhibits enhanced RNA guide binding activity, as compared to a parent polypeptide, in a buffer having a pH in a range of about 7.3 to about 8.6. In some embodiments, the variant Cas12i3 polypeptide exhibits enhanced RNA guide binding activity, as compared to a parent polypeptide, when the Tₘ value of the variant Cas12i3 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C. or 20° C. greater than the Tₘ value of a parent polypeptide. In one embodiment, the variant Cas12i3 polypeptide exhibits enhanced RNA guide binding activity when the Tₘ value of the variant Cas12i3 polypeptide is at least 8° C. greater than the Tₘ value of the parent polypeptide.

In some embodiments, at least one alteration is introduced into the parent polypeptide of SEQ ID NO: 3 to produce a variant Cas12i3 polypeptide that exhibits (a) decreased enzymatic activity and (b) enhanced RNA guide binding activity relative to the parent polypeptide of SEQ ID NO: 3. In some embodiments, at least one alteration is introduced into the parent polypeptide of SEQ ID NO: 3 to produce a variant Cas12i3 polypeptide that exhibits (a) increased enzymatic activity and (b) enhanced RNA guide binding activity, relative to the parent polypeptide of SEQ ID NO: 3. In some embodiments, at least one alteration is introduced into the parent polypeptide of SEQ ID NO: 3 to produce a variant Cas12i3 polypeptide that exhibits (a) retained enzymatic activity and (b) enhanced RNA guide binding activity, relative to the parent polypeptide of SEQ ID NO: 3.

In some embodiments, the variant Cas12i3 polypeptide comprises at least one alteration that enhances binding specificity to an RNA guide, as compared to a parent polypeptide. In some embodiments, the variant Cas12i3 polypeptide exhibits enhanced RNA guide binding specificity, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant Cas12i3 polypeptide exhibits enhanced RNA guide binding specificity, as compared to a parent polypeptide, in a buffer having a pH in a range of about 7.3 to about 8.6. In some embodiments, the variant Cas12i3 polypeptide exhibits enhanced RNA guide binding specificity, as compared to a parent polypeptide, when the Tₘ value of the variant Cas12i3 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant Cas12i3 polypeptide exhibits enhanced RNA guide binding specificity when the Tₘ value of the variant Cas12i3 polypeptide is at least 8° C. greater than the Tₘ value of the parent polypeptide.

In some embodiments, at least one alteration is introduced into the parent polypeptide of SEQ ID NO: 3 to produce a variant Cas12i3 polypeptide that exhibits (a) decreased enzymatic activity and (b) enhanced RNA guide binding specificity relative to the parent polypeptide of SEQ ID NO: 3. In some embodiments, at least one alteration is introduced into the parent polypeptide of SEQ ID NO: 3 to produce a variant Cas12i3 polypeptide that exhibits (a) increased enzymatic activity and (b) enhanced RNA guide binding specificity, relative to the parent polypeptide of SEQ ID NO: 3. In some embodiments, at least one alteration is introduced into the parent polypeptide of SEQ ID NO: 3 to produce a variant Cas12i3 polypeptide that exhibits (a) retained enzymatic activity and (b) enhanced RNA guide binding specificity, relative to the parent polypeptide of SEQ ID NO: 3.

In some embodiments, the variant Cas12i3 polypeptide comprises at least one alteration that enhances protein-RNA interactions, as compared to a parent polypeptide. In some embodiments, the variant Cas12i3 polypeptide exhibits enhanced protein-RNA interactions, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant Cas12i3 polypeptide exhibits enhanced protein-RNA interactions, as compared to a parent polypeptide, in a buffer having a pH in a range of about 7.3 to about 8.6. In some embodiments, the variant Cas12i3 polypeptide exhibits enhanced protein-RNA interactions, as compared to a parent polypeptide, when the Tₘ value of the variant Cas12i3 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant Cas12i3 polypeptide exhibits enhanced protein-RNA interactions when the Tₘ value of the variant Cas12i3 polypeptide is at least 8° C. greater than the Tₘ value of the parent polypeptide.

In some embodiments, at least one alteration is introduced into the parent polypeptide of SEQ ID NO: 3 to produce a variant Cas12i3 polypeptide that exhibits (a) decreased enzymatic activity and (b) enhanced protein-RNA interactions relative to the parent polypeptide of SEQ ID NO: 3. In some embodiments, at least one alteration is introduced into the parent polypeptide of SEQ ID NO: 3 to produce a variant Cas12i3 polypeptide that exhibits (a) increased enzymatic activity and (b) enhanced protein-RNA interactions, relative to the parent polypeptide of SEQ ID NO: 3. In some embodiments, at least one alteration is introduced into the parent polypeptide of SEQ ID NO: 3 to produce a variant Cas12i3 polypeptide that exhibits (a) retained enzymatic activity and (b) enhanced protein-RNA interactions, relative to the parent polypeptide of SEQ ID NO: 3.

In some embodiments, the variant Cas12i3 polypeptide comprises at least one alteration that enhances protein stability, as compared to a parent polypeptide. In some embodiments, the variant Cas12i3 polypeptide exhibits enhanced protein stability, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant Cas12i3 polypeptide exhibits enhanced protein stability, as compared to a parent polypeptide, in a buffer having a pH in a range of about 7.3 to about 8.6. In some embodiments, the variant Cas12i3 polypeptide exhibits enhanced protein stability, as compared to a parent polypeptide, when the Tₘ value of the variant Cas12i3 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C. or 20° C. greater than the Tₘ value of a parent polypeptide. In one embodiment, the variant Cas12i3 polypeptide exhibits enhanced protein stability when the Tₘ value of the variant Cas12i3 polypeptide is at least 8° C. greater than the Tₘ value of the parent polypeptide.

In some embodiments, at least one alteration is introduced into the parent polypeptide of SEQ ID NO: 3 to produce a variant Cas12i3 polypeptide that exhibits (a) decreased enzymatic activity and (b) enhanced protein stability relative to the parent polypeptide of SEQ ID NO: 3. In some embodiments, at least one alteration is introduced into the parent polypeptide of SEQ ID NO: 3 to produce a variant Cas12i3 polypeptide that exhibits (a) increased enzymatic activity and (b) enhanced protein stability, relative to the parent polypeptide of SEQ ID NO: 3. In some embodiments, at least one alteration is introduced into the parent polypeptide of SEQ ID NO: 3 to produce a variant Cas12i3 polypeptide that exhibits (a) retained enzymatic activity and (b) enhanced protein stability, relative to the parent polypeptide of SEQ ID NO: 3.

In some embodiments, the variant Cas12i3 polypeptide comprises at least one alteration that decreases dissociation from an RNA guide (e.g., binary complex dissociation), as compared to a parent polypeptide. In some embodiments, the variant Cas12i3 polypeptide exhibits decreased dissociation from an RNA guide, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant Cas12i3 polypeptide exhibits decreased dissociation from an RNA guide, as compared to a parent polypeptide, in a buffer having a pH in a range of about 7.3 to about 8.6. In some embodiments, the variant Cas12i3 polypeptide exhibits decreased dissociation from an RNA guide, as compared to a parent polypeptide, when the Tₘ value of the variant Cas12i3 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C. or 20° C. greater than the Tₘ value of a parent polypeptide. In one embodiment, the variant Cas12i3 polypeptide exhibits decreased dissociation from an RNA guide when the Tₘ value of the variant Cas12i3 polypeptide is at least 8° C. greater than the Tₘ value of the parent polypeptide. In some embodiments, the variant Cas12i3 polypeptide exhibits decreased dissociation from an RNA guide, as compared to a parent polypeptide, over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours. In some embodiments, a variant effector ribonucleoprotein (RNP) complex does not exchange the RNA guide with a different RNA.

In some embodiments, at least one alteration is introduced into the parent polypeptide of SEQ ID NO: 3 to produce a variant Cas12i3 polypeptide that exhibits (a) decreased enzymatic activity and (b) decreased dissociation from an RNA guide relative to the parent polypeptide of SEQ ID NO: 3. In some embodiments, at least one alteration is introduced into the parent polypeptide of SEQ ID NO: 3 to produce a variant Cas12i3 polypeptide that exhibits (a) increased enzymatic activity and (b) decreased dissociation from an RNA guide, relative to the parent polypeptide of SEQ ID NO: 3. In some embodiments, at least one alteration is introduced into the parent polypeptide of SEQ ID NO: 3 to produce a variant Cas12i3 polypeptide that exhibits (a) retained enzymatic activity and (b) decreased dissociation from an RNA guide, relative to the parent polypeptide of SEQ ID NO: 3.

In some embodiments, the variant Cas12i3 polypeptide comprises at least one alteration that enhances ternary complex formation with an RNA guide and a target nucleic acid, as compared to a parent polypeptide. In some embodiments, the variant Cas12i3 polypeptide exhibits enhanced ternary complex formation, as compared to a parent polypeptide, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant Cas12i3 polypeptide exhibits enhanced ternary complex formation, as compared to a parent polypeptide, in a buffer having a pH in a range of about 7.3 to about 8.6. In some embodiments, the variant Cas12i3 polypeptide exhibits enhanced ternary complex formation, as compared to a parent polypeptide, when the Tₘ value of the variant Cas12i3 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C. or 20° C. greater than the Tₘ value of a parent polypeptide. In one embodiment, the variant Cas12i3 polypeptide exhibits enhanced ternary complex formation when the Tₘ value of the variant Cas12i3 polypeptide is at least 8° C. greater than the Tₘ value of the parent polypeptide.

In some embodiments, at least one alteration is introduced into the parent polypeptide of SEQ ID NO: 3 to produce a variant Cas12i3 polypeptide that exhibits (a) decreased enzymatic activity and (b) enhanced ternary complex formation relative to the parent polypeptide of SEQ ID NO: 3. In some embodiments, at least one alteration is introduced into the parent polypeptide of SEQ ID NO: 3 to produce a variant Cas12i3 polypeptide that exhibits (a) increased enzymatic activity and (b) enhanced ternary complex formation, relative to the parent polypeptide of SEQ ID NO: 3. In some embodiments, at least one alteration is introduced into the parent polypeptide of SEQ ID NO: 3 to produce a variant Cas12i3 polypeptide that exhibits (a) retained enzymatic activity and (b) enhanced ternary complex formation, relative to the parent polypeptide of SEQ ID NO: 3.

In some embodiments, the variant Cas12i3 polypeptide comprises at least one alteration such that a binary complex comprising the variant Cas12i3 polypeptide (e.g., a variant binary complex) exhibits enhanced binding affinity to a target nucleic acid, as compared to a parent binary complex. In some embodiments, the variant binary complex exhibits enhanced binding affinity to a target nucleic acid, as compared to a parent binary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant binary complex exhibits enhanced binding affinity to a target nucleic acid, as compared to a parent binary complex, in a buffer having a pH in a range of about 7.3 to about 8.6. In some embodiments, the variant binary complex exhibits enhanced binding affinity to a target nucleic acid, as compared to a parent binary complex, when the Tₘ value of the variant binary complex is at least 1° C., 2° C., 3° C., 4° C., 5° C., 6° C., 7° C., 8° C., 9°° C., 10° C., 11° C., 12° C., 13° C., 14° C., 15° C., 16° C., 17° C., 18° C., 19° C. or 20° C. greater than the Tₘ value of a parent binary complex. In one embodiment, the variant binary complex exhibits enhanced binding affinity to a target nucleic acid when the Tₘ value of the variant binary complex is at least 8° C. greater than the Tₘ value of the parent binary complex.

In some embodiments, at least one alteration is introduced into the parent polypeptide of SEQ ID NO: 3 to produce a variant Cas12i3 polypeptide that forms a variant binary complex exhibiting (a) decreased enzymatic activity and (b) enhanced binding affinity to a target nucleic acid, relative to a parent binary complex comprising the polypeptide of SEQ ID NO: 3. In some embodiments, at least one alteration is introduced into the parent polypeptide of SEQ ID NO: 3 to produce a variant Cas12i3 polypeptide that forms a variant binary complex exhibiting (a) increased enzymatic activity and (b) enhanced binding affinity to a target nucleic acid, relative to a parent binary complex comprising the polypeptide of SEQ ID NO: 3. In some embodiments, at least one alteration is introduced into the parent polypeptide of SEQ ID NO: 3 to produce a variant Cas12i3 polypeptide that forms a variant binary complex that exhibits (a) retained enzymatic activity and (b) enhanced binding affinity to a target nucleic acid, relative to a parent binary complex comprising the polypeptide of SEQ ID NO: 3.

In some embodiments, the variant Cas12i3 polypeptide comprises at least one alteration such that a binary complex comprising the variant Cas12i3 polypeptide (e.g., a variant binary complex) exhibits enhanced on-target binding activity, as compared to a parent binary complex. In some embodiments, the variant binary complex exhibits enhanced on-target binding activity, as compared to a parent binary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant binary complex exhibits enhanced on-target binding activity, as compared to a parent binary complex, in a buffer having a pH in a range of about 7.3 to about 8.6. In some embodiments, the variant binary complex exhibits enhanced on-target binding activity, as compared to a parent binary complex, when the Tₘ value of the variant binary complex is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent binary complex. In one embodiment, the variant binary complex exhibits enhanced on-target binding activity when the Tₘ value of the variant binary complex is at least 8° C. greater than the Tₘ value of the parent binary complex.

In some embodiments, at least one alteration is introduced into the parent polypeptide of SEQ ID NO: 3 to produce a variant Cas12i3 polypeptide that forms a variant binary complex exhibiting (a) decreased enzymatic activity and (b) enhanced on-target binding activity, relative to a parent binary complex comprising the polypeptide of SEQ ID NO: 3. In some embodiments, at least one alteration is introduced into the parent polypeptide of SEQ ID NO: 3 to produce a variant Cas12i3 polypeptide that forms a variant binary complex exhibiting (a) increased enzymatic activity and (b) enhanced on-target binding activity, relative to a parent binary complex comprising the polypeptide of SEQ ID NO: 3. In some embodiments, at least one alteration is introduced into the parent polypeptide of SEQ ID NO: 3 to produce a variant Cas12i3 polypeptide that forms a variant binary complex that exhibits (a) retained enzymatic activity and (b) enhanced on-target binding activity, relative to a parent binary complex comprising the polypeptide of SEQ ID NO: 3.

In some embodiments, the variant Cas12i3 polypeptide comprises at least one alteration such that a binary complex comprising the variant Cas12i3 polypeptide (e.g., a variant binary complex) exhibits enhanced on-target binding specificity, as compared to a parent binary complex. In some embodiments, the variant binary complex exhibits enhanced on-target binding specificity, as compared to a parent binary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant binary complex exhibits enhanced on-target binding specificity, as compared to a parent binary complex, in a buffer having a pH in a range of about 7.3 to about 8.6. In some embodiments, the variant binary complex exhibits enhanced on-target binding specificity, as compared to a parent binary complex, when the Tₘ value of the variant binary complex is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent binary complex. In one embodiment, the variant binary complex exhibits enhanced on-target binding specificity when the Tₘ value of the variant binary complex is at least 8° C. greater than the Tₘ value of the parent binary complex.

In some embodiments, at least one alteration is introduced into the parent polypeptide of SEQ ID NO: 3 to produce a variant Cas12i3 polypeptide that forms a variant binary complex exhibiting (a) decreased enzymatic activity and (b) enhanced on-target binding specificity, relative to a parent binary complex comprising the polypeptide of SEQ ID NO: 3. In some embodiments, at least one alteration is introduced into the parent polypeptide of SEQ ID NO: 3 to produce a variant Cas12i3 polypeptide that forms a variant binary complex exhibiting (a) increased enzymatic activity and (b) enhanced on-target binding specificity, relative to a parent binary complex comprising the polypeptide of SEQ ID NO: 3. In some embodiments, at least one alteration is introduced into the parent polypeptide of SEQ ID NO: 3 to produce a variant Cas12i3 polypeptide that forms a variant binary complex that exhibits (a) retained enzymatic activity and (b) enhanced on-target binding specificity, relative to a parent binary complex comprising the polypeptide of SEQ ID NO: 3.

In some embodiments, the variant Cas12i3 polypeptide comprises at least one alteration such that a binary complex comprising the variant Cas12i3 polypeptide (e.g., a variant binary complex) exhibits decreased off-target binding to a non-target nucleic acid, as compared to a parent binary complex. In some embodiments, the variant binary complex exhibits decreased off-target binding to a non-target nucleic acid, as compared to a parent binary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant binary complex exhibits decreased off-target binding to a non-target nucleic acid, as compared to a parent binary complex, in a buffer having a pH in a range of about 7.3 to about 8.6. In some embodiments, the variant binary complex exhibits decreased off-target binding to a non-target nucleic acid, as compared to a parent binary complex, when the Tₘ value of the variant Cas12i3 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant binary complex exhibits decreased off-target binding to a non-target nucleic acid when the Tₘ value of the variant binary complex is at least 8° C. greater than the Tₘ value of the parent polypeptide.

In some embodiments, at least one alteration is introduced into the parent polypeptide of SEQ ID NO: 3 to produce a variant Cas12i3 polypeptide that forms a variant binary complex exhibiting (a) decreased enzymatic activity and (b) decreased off-target binding to a non-target nucleic acid, relative to a parent binary complex comprising the polypeptide of SEQ ID NO: 3. In some embodiments, at least one alteration is introduced into the parent polypeptide of SEQ ID NO: 3 to produce a variant Cas12i3 polypeptide that forms a variant binary complex exhibiting (a) increased enzymatic activity and (b) decreased off-target binding to a non-target nucleic acid, relative to a parent binary complex comprising the polypeptide of SEQ ID NO: 3. In some embodiments, at least one alteration is introduced into the parent polypeptide of SEQ ID NO: 3 to produce a variant Cas12i3 polypeptide that forms a variant binary complex exhibiting (a) retained enzymatic activity and (b) decreased off-target binding to a non-target nucleic acid, relative to a parent binary complex comprising the polypeptide of SEQ ID NO: 3.

In some embodiments, the variant Cas12i3 polypeptide comprises at least one alteration such that a binary complex comprising the variant Cas12i3 polypeptide (e.g., a variant binary complex) exhibits decreased dissociation from the target nucleic acid, as compared to a parent binary complex. In some embodiments, the variant binary complex exhibits decreased dissociation from the target nucleic acid, as compared to a parent binary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant binary complex exhibits decreased dissociation from the target nucleic acid, as compared to a parent binary complex, in a buffer having a pH in a range of about 7.3 to about 8.6. In some embodiments, the variant binary complex exhibits decreased dissociation from the target nucleic acid, as compared to a parent binary complex, when the Tₘ value of the variant Cas12i3 polypeptide is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent polypeptide. In one embodiment, the variant binary complex exhibits decreased dissociation from the target nucleic acid when the Tₘ value of the variant binary complex is at least 8° C. greater than the Tₘ value of the parent polypeptide.

In some embodiments, at least one alteration is introduced into the parent polypeptide of SEQ ID NO: 3 to produce a variant Cas12i3 polypeptide that forms a variant binary complex exhibiting (a) decreased enzymatic activity and (b) decreased dissociation from the target nucleic acid, relative to a parent binary complex comprising the polypeptide of SEQ ID NO: 3. In some embodiments, at least one alteration is introduced into the parent polypeptide of SEQ ID NO: 3 to produce a variant Cas12i3 polypeptide that forms a variant binary complex exhibiting (a) increased enzymatic activity and (b) decreased dissociation from the target nucleic acid, relative to a parent binary complex comprising the polypeptide of SEQ ID NO: 3. In some embodiments, at least one alteration is introduced into the parent polypeptide of SEQ ID NO: 3 to produce a variant Cas12i3 polypeptide that forms a variant binary complex exhibiting (a) retained enzymatic activity and (b) decreased dissociation from the target nucleic acid, relative to a parent binary complex comprising the polypeptide of SEQ ID NO: 3.

In some embodiments, the variant Cas12i3 polypeptide comprises at least one alteration such that a ternary complex comprising the variant Cas12i3 polypeptide (e.g., a variant ternary complex) exhibits enhanced stability, as compared to a parent ternary complex. In some embodiments, the variant ternary complex exhibits enhanced stability, as compared to a parent ternary complex, at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., 51° C., 52° C., 53° C., 54° C., 55° C., 56° C., 57° C., 58° C., 59° C., 60° C. or 65° C. In some embodiments, the variant ternary complex exhibits enhanced stability, as compared to a parent ternary complex, in a buffer having a pH in a range of about 7.3 to about 8.6. In some embodiments, the variant ternary complex exhibits enhanced stability, as compared to a parent ternary complex, when the Tₘ value of the variant ternary complex is at least 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., or 20° C. greater than the Tₘ value of a parent ternary complex. In one embodiment, the variant ternary complex exhibits enhanced stability when the Tₘ value of the variant ternary complex is at least 8° C. greater than the Tₘ value of the parent ternary complex.

Although the changes described herein may be one or more amino acid changes, changes to the variant Cas12i3 polypeptide may also be of a substantive nature, such as fusion of polypeptides as amino- and/or carboxyl-terminal extensions. For example, variant Cas12i3 polypeptide may contain additional peptides, e.g., one or more peptides. Examples of additional peptides may include epitope peptides for labelling, such as a polyhistidine tag (His-tag), Myc, and FLAG. In some embodiments, the variant Cas12i3 polypeptide described herein can be fused to a detectable moiety such as a fluorescent protein (e.g., green fluorescent protein (GFP) or yellow fluorescent protein (YFP)).

In some embodiments, the variant Cas12i3 polypeptide comprises at least one (e.g., two, three, four, five, six, or more) nuclear localization signal (NLS). In some embodiments, the variant Cas12i3 polypeptide comprises at least one (e.g., two, three, four, five, six, or more) nuclear export signal (NES). In some embodiments, the variant Cas12i3 polypeptide comprises at least one (e.g., two, three, four, five, six, or more) NLS and at least one (e.g., two, three, four, five, six, or more) NES.

In some embodiments, the variant Cas12i3 polypeptide described herein can be self-inactivating. See. Epstein et al., “Engineering a Self-Inactivating CRISPR System for AAV Vectors,” Mol. Ther., 24 (2016): S50, which is incorporated by reference in its entirety.

In some embodiments, the nucleotide sequence encoding the variant Cas12i3 polypeptide described herein can be codon-optimized for use in a particular host cell or organism. For example, the nucleic acid can be codon-optimized for any non-human eukaryote including mice, rats, rabbits, dogs, livestock, or non-human primates. Codon usage tables are readily available, for example, at the “Codon Usage Database” available at www.kazusa.orjp/codon/and these tables can be adapted in a number of ways. See Nakamura et al. Nucl. Acids Res. 28:292 (2000), which is incorporated herein by reference in its entirety. Computer algorithms for codon optimizing a particular sequence for expression in a particular host cell are also available, such as Gene Forge (Aptagen; Jacobus, PA).

### RNA Guide

In some embodiments, a composition or gene editing system, or a complex as described herein comprises a targeting moiety (e.g., an RNA guide, antisense, oligonucleotides, peptide oligonucleotide conjugates) that binds the target nucleic acid and interacts with the variant Cas12i3 polypeptide. The targeting moiety may bind a target nucleic acid (e.g., with specific binding affinity to the target nucleic acid).

In some embodiments, the targeting moiety comprises, or is, an RNA guide. In some embodiments, the RNA guide directs the variant Cas12i3 polypeptide described herein to a particular nucleic acid sequence. Those skilled in the art reading the below examples of particular kinds of RNA guides will understand that, in some embodiments, an RNA guide is site-specific. That is, in some embodiments, an RNA guide associates specifically with one or more target nucleic acid sequences (e.g., specific DNA or genomic DNA sequences) and not to non-targeted nucleic acid sequences (e.g., non-specific DNA or random sequences).

In some embodiments, the composition or gene editing system as described herein comprises an RNA guide that associates with the variant Cas12i3 polypeptide described herein and directs the variant Cas12i3 polypeptide to a target nucleic acid sequence (e.g., DNA).

The RNA guide may target (e.g., associate with, be directed to, contact, or bind) one or more nucleotides of a target sequence, e.g., a site-specific sequence or a site-specific target. In some embodiments, the variant effector nucleoprotein (e.g., variant Cas12i3 polypeptide plus an RNA guide) is activated upon binding to a target nucleic acid that is complementary to a DNA-targeting sequence in the RNA guide (e.g., a sequence-specific substrate or target nucleic acid).

In some embodiments, an RNA guide comprises a spacer having a length of from about 11 nucleotides to about 100 nucleotides. For example, the DNA-targeting segment can have a length of from about 11 nucleotides to about 80 nucleotides, from about 11 nucleotides to about 50 nucleotides, from about 11 nucleotides to about 40 nucleotides, from about 11 nucleotides to about 30 nucleotides, from about 11 nucleotides to about 25 nucleotides, from about 11 nucleotides to about 20 nucleotides, or from about 11 nucleotides to about 19 nucleotides. For example, the spacer can have a length of from about 19 nucleotides to about 20 nucleotides, from about 19 nucleotides to about 25 nucleotides, from about 19 nucleotides to about 30 nucleotides, from about 19 nucleotides to about 35 nucleotides, from about 19 nucleotides to about 40 nucleotides, from about 19 nucleotides to about 45 nucleotides, from about 19 nucleotides to about 50 nucleotides, from about 19 nucleotides to about 60 nucleotides, from about 19 nucleotides to about 70 nucleotides, from about 19 nucleotides to about 80 nucleotides, from about 19 nucleotides to about 90 nucleotides, from about 19 nucleotides to about 100 nucleotides, from about 20 nucleotides to about 25 nucleotides, from about 20 nucleotides to about 30 nucleotides, from about 20 nucleotides to about 35 nucleotides, from about 20 nucleotides to about 40 nucleotides, from about 20 nucleotides to about 45 nucleotides, from about 20 nucleotides to about 50 nucleotides, from about 20 nucleotides to about 60 nucleotides, from about 20 nucleotides to about 70 nucleotides, from about 20 nucleotides to about 80 nucleotides, from about 20 nucleotides to about 90 nucleotides, or from about 20 nucleotides to about 100 nucleotides.

In some embodiments, the spacer of the RNA guide may be generally designed to have a length of between 11 and 50 nucleotides (e.g., 11, 12, 13, 14, 15, 16, 17, 18, 19, 20, 21, 22, 23, 24, 25, 26, 27, 28, 29, 30, 31, 32, 33, 34, or 35 nucleotides) and be complementary to a specific target nucleic acid sequence (e.g., the complement of a “target sequence” as described herein). In some particular embodiments, the RNA guide may be designed to be complementary to a specific DNA strand, e.g., of a genomic locus. In some embodiments, the DNA targeting sequence is designed to be complementary to a specific DNA strand, e.g., of a genomic locus.

The RNA guide may be substantially identical to a complementary strand of a reference nucleic acid sequence. In some embodiments, the RNA guide comprises a sequence having least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a reference nucleic acid sequence, e.g., target nucleic acid. The percent identity between two such nucleic acids can be determined manually by inspection of the two optimally aligned nucleic acid sequences or by using software programs or algorithms (e.g., BLAST, ALIGN, CLUSTAL) using standard parameters.

In some embodiments, the RNA guide has at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a complementary strand of a target nucleic acid.

In some embodiments, the RNA guide comprises a spacer that is a length of between 11 and 50 nucleotides (e.g., 11, 12, 13, 14, 15, 16, 17, 18, 19, 20, 21, 22, 23, 24, 25, 26, 27, 28, 29, 30, 31, 32, 33, 34, or 35 nucleotides) and at least 80%, at least 90%, at least 95%, at least 96%, at least 97%, at least 98%, at least 99% complementary to a target nucleic acid. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence. In some embodiments, the RNA guide comprises a sequence, e.g., RNA sequence, that is a length of up to 50 and at least 80%, at least 90%, at least 95%, at least 96%, at least 97%, at least 98%, at least 99% complementary to a target nucleic acid. In some embodiments, the RNA guide comprises a sequence at least 80%, at least 90%, at least 95%, at least 96%, at least 97%, at least 98%, at least 99% complementary to a target DNA sequence. In some embodiments, the RNA guide comprises a sequence at least 80%, at least 90%, at least 95%, at least 96%, at least 97%, at least 98%, at least 99% complementary to a target genomic sequence.

In certain embodiments, the RNA guide includes, consists essentially of, or comprises a direct repeat sequence linked to a DNA targeting sequence. In some embodiments, the RNA guide includes a direct repeat sequence and a DNA targeting sequence or a direct repeat-DNA targeting sequence-direct repeat sequence. In some embodiments, the RNA guide includes a truncated direct repeat sequence and a DNA targeting sequence, which is typical of processed or mature crRNA. In some embodiments, the variant Cas12i3 polypeptide described herein forms a complex with the RNA guide, and the RNA guide directs the complex to associate with site-specific target nucleic acid that is complementary to at least a portion of the RNA guide.

In some embodiments, the direct repeat sequence is at least 90% identical to the following sequences: CUAGCAAUGACCUAAUAGUGUGUCCUUAGUUGACAU (SEQ ID NO: 4), CCUACAAUACCUAAGAAAUCCGUCCUAAGUUGACGG (SEQ ID NO: 5), or AUAGUGUGUCCUUAGUUGACAU (SEQ ID NO: 6). In some embodiments, the direct repeat sequence is at least 95% identical to a sequence set forth in SEQ ID NO: 4. SEQ ID NO: 5, or SEQ ID NO: 6. In some embodiments, the direct repeat is 100% identical to a sequence set forth in SEQ ID NO: 4, SEQ ID NO: 5, or SEQ ID NO: 6.

The direct repeat sequence can comprise nucleotide 1 through nucleotide 36 of SEQ ID NO: 4 or SEQ ID NO: 5. The direct repeat sequence can comprise nucleotide 2 through nucleotide 36 of SEQ ID NO: 4 or SEQ ID NO: 5. The direct repeat sequence can comprise nucleotide 3 through nucleotide 36 of SEQ ID NO: 4 or SEQ ID NO: 5. The direct repeat sequence can comprise nucleotide 4 through nucleotide 36 of SEQ ID NO: 4 or SEQ ID NO: 5. The direct repeat sequence can comprise nucleotide 5 through nucleotide 36 of SEQ ID NO: 4 or SEQ ID NO: 5. The direct repeat sequence can comprise nucleotide 6 through nucleotide 36 of SEQ ID NO: 4 or SEQ ID NO: 5. The direct repeat sequence can comprise nucleotide 7 through nucleotide 36 of SEQ ID NO: 4 or SEQ ID NO: 5. The direct repeat sequence can comprise nucleotide 8 through nucleotide 36 of SEQ ID NO: 4 or SEQ ID NO: 5. The direct repeat sequence can comprise nucleotide 9 through nucleotide 36 of SEQ ID NO: 4 or SEQ ID NO: 5. The direct repeat sequence can comprise nucleotide 10 through nucleotide 36 of SEQ ID NO: 4 or SEQ ID NO: 5. The direct repeat sequence can comprise nucleotide 11 through nucleotide 36 of SEQ ID NO: 4 or SEQ ID NO: 5. The direct repeat sequence can comprise nucleotide 12 through nucleotide 36 of SEQ ID NO: 4 or SEQ ID NO: 5. The direct repeat sequence can comprise nucleotide 13 through nucleotide 36 of SEQ ID NO: 4 or SEQ ID NO: 5. The direct repeat sequence can comprise nucleotide 14 through nucleotide 36 of SEQ ID NO: 4 or SEQ ID NO: 5.

The direct repeat sequence can have at least 95% identity to a sequence comprising nucleotide 1 through nucleotide 36 of SEQ ID NO: 4 or SEQ ID NO: 5. The direct repeat sequence can have at least 95% identity to a sequence comprising 2 through nucleotide 36 of SEQ ID NO: 4 or SEQ ID NO: 5. The direct repeat sequence can have at least 95% identity to a sequence comprising 3 through nucleotide 36 of SEQ ID NO: 4 or SEQ ID NO: 5. The direct repeat sequence can have at least 95% identity to a sequence comprising 4 through nucleotide 36 of SEQ ID NO: 4 or SEQ ID NO: 5. The direct repeat sequence can have at least 95% identity to a sequence comprising 5 through nucleotide 36 of SEQ ID NO: 4 or SEQ ID NO: 5. The direct repeat sequence can have at least 95% identity to a sequence comprising 6 through nucleotide 36 of SEQ ID NO: 4 or SEQ ID NO: 5. The direct repeat sequence can have at least 95% identity to a sequence comprising 7 through nucleotide 36 of SEQ ID NO: 4 or SEQ ID NO: 5. The direct repeat sequence can have at least 95% identity to a sequence comprising 8 through nucleotide 36 of SEQ ID NO: 4 or SEQ ID NO: 5. The direct repeat sequence can have at least 95% identity to a sequence comprising 9 through nucleotide 36 of SEQ ID NO: 4 or SEQ ID NO: 5. The direct repeat sequence can have at least 95% identity to a sequence comprising 10 through nucleotide 36 of SEQ ID NO: 4 or SEQ ID NO: 5. The direct repeat sequence can have at least 95% identity to a sequence comprising 11 through nucleotide 36 of SEQ ID NO: 4 or SEQ ID NO: 5. The direct repeat sequence can have at least 95% identity to a sequence comprising 12 through nucleotide 36 of SEQ ID NO: 4 or SEQ ID NO: 5. The direct repeat sequence can have at least 95% identity to a sequence comprising 13 through nucleotide 36 of SEQ ID NO: 4 or SEQ ID NO: 5. The direct repeat sequence can have at least 95% identity to a sequence comprising 14 through nucleotide 36 of SEQ ID NO: 4 or SEQ ID NO: 5.

The direct repeat sequence can have at least 90% identity to a sequence comprising nucleotide 1 through nucleotide 36 of SEQ ID NO: 4 or SEQ ID NO: 5. The direct repeat sequence can have at least 90% identity to a sequence comprising 2 through nucleotide 36 of SEQ ID NO: 4 or SEQ ID NO: 5. The direct repeat sequence can have at least 90% identity to a sequence comprising 3 through nucleotide 36 of SEQ ID NO: 4 or SEQ ID NO: 5. The direct repeat sequence can have at least 90% identity to a sequence comprising 4 through nucleotide 36 of SEQ ID NO: 4 or SEQ ID NO: 5. The direct repeat sequence can have at least 90% identity to a sequence comprising 5 through nucleotide 36 of SEQ ID NO: 4 or SEQ ID NO: 5. The direct repeat sequence can have at least 90% identity to a sequence comprising 6 through nucleotide 36 of SEQ ID NO: 4 or SEQ ID NO: 5. The direct repeat sequence can have at least 90% identity to a sequence comprising 7 through nucleotide 36 of SEQ ID NO: 4 or SEQ ID NO: 5. The direct repeat sequence can have at least 90% identity to a sequence comprising 8 through nucleotide 36 of SEQ ID NO: 4 or SEQ ID NO: 5. The direct repeat sequence can have at least 90% identity to a sequence comprising 9 through nucleotide 36 of SEQ ID NO: 4 or SEQ ID NO: 5. The direct repeat sequence can have at least 90% identity to a sequence comprising 10 through nucleotide 36 of SEQ ID NO: 4 or SEQ ID NO: 5. The direct repeat sequence can have at least 90% identity to a sequence comprising 11 through nucleotide 36 of SEQ ID NO: 4 or SEQ ID NO: 5. The direct repeat sequence can have at least 90% identity to a sequence comprising 12 through nucleotide 36 of SEQ ID NO: 4 or SEQ ID NO: 5. The direct repeat sequence can have at least 90% identity to a sequence comprising 13 through nucleotide 36 of SEQ ID NO: 4 or SEQ ID NO: 5. The direct repeat sequence can have at least 90% identity to a sequence comprising 14 through nucleotide 36 of SEQ ID NO: 4 or SEQ ID NO: 5.

In some embodiments, the direct repeat sequence is at least 90% identical to the reverse complement of any one of SEQ ID NOs: 4-6. In some embodiments, the direct repeat sequence is at least 95% identical to the reverse complement of any one of SEQ ID NOs: 4-6. In some embodiments, the direct repeat sequence is the reverse complement of any one of SEQ ID NOs: 4-6.

In some embodiments, PAMs corresponding to variant Cas12i3 polypeptide of the present invention include 5′-TTN-3′ and 5′-NTTN-3′. As used herein, N's can each be any nucleotide (e.g., A, G, T, or C) or a subset thereof (e.g., Y (C or T), K (G or T), B (G, T, or C), H (A, C, or T). In some embodiments, a variant Cas12i3 polypeptide of the present invention binds to a target nucleic acid adjacent to a 5′-TTN-3′ or 5′-NTTN-3′ sequence.

In some embodiments, the composition or gene editing system, or the complex described herein includes one or more (e.g., two, three, four, five, six, seven, eight, or more) RNA guides, e.g., a plurality of RNA guides.

In some embodiments, the RNA guide has an architecture similar to, for example International Publication Nos. WO 2014/093622 and WO 2015/070083, the entire contents of each of which are incorporated herein by reference.

Unless otherwise noted, all compositions or gene editing systems, complexes, and polypeptides provided herein are made in reference to the active level of that composition or gene editing system, or complex or polypeptide, and are exclusive of impurities, for example, residual solvents or by-products, which may be present in commercially available sources. Enzymatic component weights are based on total active protein. All percentages and ratios are calculated by weight unless otherwise indicated. All percentages and ratios are calculated based on the total composition or gene editing system unless otherwise indicated. In the exemplified composition, the enzymatic levels are expressed by pure enzyme by weight of the total composition or gene editing system and unless otherwise specified, the ingredients are expressed by weight of the total compositions or gene editing systems.

### Modifications

The RNA guide or any of the nucleic acid sequences encoding the variant Cas12i3 polypeptides may include one or more covalent modifications with respect to a reference sequence, in particular the parent polyribonucleotide, which are included within the scope of this invention.

Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide or any of the nucleic acid sequences encoding components of the variant Cas12i3 polypeptides may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A. G. U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, as phosphoramidates such 3′-amino phosphoramidate and aminoalkylphosphoramidates, thionophosphoramidates, thionoalkylphosphonates, thionoalkylphosphotriesters, and boranophosphates having normal 3′-5′ linkages. 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine. 5′-O-(1-thiophosphate)-cytidine (a-thio-cytidine). 5′-O-(1-thiophosphate)-guanosine. 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine. 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2.4 (1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J. Crain, P. and McCloskey. J. (1999). The RNA Modification Database: 1999 update. Nucl Acids Res 27:196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine. 2-thiouridine, 4-thio-pseudouridine. 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, 2-dihydrouridine, dihydropseudouridine, 2-thio-dihydrouridine, 2-thio-dihydropseudouridine, methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

### Target Nucleic Acid

The methods disclosed herein are applicable for a variety of target nucleic acids. In some embodiments, the target nucleic acid is a DNA, such as a DNA locus. In some embodiments, the target nucleic acid is an RNA, such as an RNA locus or mRNA. In some embodiments, the target nucleic acid is single-stranded (e.g., single-stranded DNA). In some embodiments, the target nucleic acid is double-stranded (e.g., double-stranded DNA). In some embodiments, the target nucleic acid comprises both single-stranded and double-stranded regions. In some embodiments, the target nucleic acid is linear. In some embodiments, the target nucleic acid is circular. In some embodiments, the target nucleic acid comprises one or more modified nucleotides, such as methylated nucleotides, damaged nucleotides, or nucleotides analogs. In some embodiments, the target nucleic acid is not modified.

The target nucleic acid may be of any length, such as about at least any one of 100 bp, 200 bp, 500 bp, 1000 bp, 2000 bp, 5000 bp, 10 kb, 20 kb, 50 kb, 100 kb, 200 kb, 500 kb, 1 Mb, or longer. The target nucleic acid may also comprise any sequence. In some embodiments, the target nucleic acid is GC-rich, such as having at least about any one of 40%, 45%, 50%, 55%, 60%, 65%, or higher GC content. In some embodiments, the target nucleic acid has a GC content of at least about 70%, 80%, or more. In some embodiments, the target nucleic acid is a GC-rich fragment in a non-GC-rich target nucleic acid. In some embodiments, the target nucleic acid is not GC-rich. In some embodiments, the target nucleic acid has one or more secondary structures or higher-order structures. In some embodiments, the target nucleic acid is not in a condensed state, such as in a chromatin, to render the target nucleic acid inaccessible by the variant Cas12i3 polypeptide/RNA guide complex.

In some embodiments, the target nucleic acid is present in a cell. In some embodiments, the target nucleic acid is present in the nucleus of the cell. In some embodiments, the target nucleic acid is endogenous to the cell. In some embodiments, the target nucleic acid is a genomic DNA. In some embodiments, the target nucleic acid is a chromosomal DNA. In one embodiment, the target nucleic acid is an extrachromosomal nucleic acid. In some embodiments, the target nucleic acid is a protein-coding gene or a functional region thereof, such as a coding region, or a regulatory element, such as a promoter, enhancer, a 5′ or 3′ untranslated region, etc. In some embodiments, the target nucleic acid is a non-coding gene, such as transposon, miRNA, tRNA, ribosomal RNA, ribozyme, or lincRNA. In some embodiments, the target nucleic acid is a plasmid.

In some embodiments, the target nucleic acid is exogenous to a cell. In some embodiments, the target nucleic acid is a viral nucleic acid, such as viral DNA or viral RNA. In some embodiments, the target nucleic acid is a horizontally transferred plasmid. In some embodiments, the target nucleic acid is integrated in the genome of the cell. In some embodiments, the target nucleic acid is not integrated in the genome of the cell. In some embodiments, the target nucleic acid is a plasmid in the cell. In some embodiments, the target nucleic acid is present in an extrachromosomal array.

In some embodiments, the target nucleic acid is an isolated nucleic acid, such as an isolated DNA or an isolated RNA. In some embodiments, the target nucleic acid is present in a cell-free environment. In some embodiments, the target nucleic acid is an isolated vector, such as a plasmid. In some embodiments, the target nucleic acid is an ultrapure plasmid.

The complement of the target sequence is a segment of the target nucleic acid that hybridizes to the RNA guide. In some embodiments, the target nucleic acid has only one copy of the complement of the target sequence. In some embodiments, the target nucleic acid has more than one copy, such as at least about any one of 2, 3, 4, 5, 10, 100, or more copies of the complement of the target sequence. For example, a target nucleic acid comprising a repeated sequence in a genome of a viral nucleic acid or a bacterium may be targeted by the variant effector nucleoprotein.

In some embodiments, the complement of the target sequence is present in a readily accessible region of the target nucleic acid. In some embodiments, the target nucleic acid is in an exon of a target gene. In some embodiments, the target nucleic acid is across an exon-intron junction of a target gene. In some embodiments, the target nucleic acid is present in a non-coding region, such as a regulatory region of a gene. In some embodiments, wherein the target nucleic acid is exogenous to a cell, the target nucleic acid comprises a sequence that is not found in the genome of the cell.

Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target nucleic acid that is complementary to and hybridizes with the RNA guide is referred to as the “target strand,” “non-PAM strand,” or “complementary strand” and the strand of the target nucleic acid that is complementary to the “target strand.” “non-PAM strand,” or “complementary strand” (and is therefore not complementary to the RNA guide) is referred to as the “non-target strand,” “non-PAM strand,” “noncomplementary strand,” or “non-complementary strand”.

### Preparation

In some embodiments, the variant Cas12i3 polypeptide of the present invention can be prepared by (a) culturing bacteria which produce the variant Cas12i3 polypeptide of the present invention, isolating the variant Cas12i3 polypeptide, optionally, purifying the variant Cas12i3 polypeptide, and complexing the variant Cas12i3 polypeptide with RNA guide. The variant Cas12i3 polypeptide can be also prepared by (b) a known genetic engineering technique, specifically, by isolating a gene encoding the variant Cas12i3 polypeptide of the present invention from bacteria, constructing a recombinant expression vector, and then transferring the vector into an appropriate host cell that expresses the RNA guide for expression of a recombinant protein that complexes with the RNA guide in the host cell. Alternatively, the variant Cas12i3 polypeptide can be prepared by (c) an in vitro coupled transcription-translation system and then complexes with RNA guide. Bacteria that can be used for preparation of the variant Cas12i3 polypeptide of the present invention are not particularly limited as long as they can produce the variant Cas12i3 polypeptide of the present invention. Some nonlimiting examples of the bacteria include E. coli cells described herein.

### Vectors

The present invention provides a vector for expressing the variant Cas12i3 polypeptide described herein or nucleic acids encoding the variant described herein may be incorporated into a vector. In some embodiments, a vector of the invention includes a nucleotide sequence encoding variant Cas12i3 polypeptide. In some embodiments, a vector of the invention includes a nucleotide sequence encoding the variant Cas12i3 polypeptide.

The present invention also provides a vector that may be used for preparation of the variant Cas12i3 polypeptide or compositions or gene editing systems comprising the variant Cas12i3 polypeptide as described herein. In some embodiments, the invention includes the composition or gene editing system, or vector described herein in a cell. In some embodiments, the invention includes a method of expressing the composition or gene editing system comprising the variant Cas12i3 polypeptide, or vector or nucleic acid encoding the variant Cas12i3 polypeptide, in a cell. The method may comprise the steps of providing the composition or gene editing system, e.g., vector or nucleic acid, and delivering the composition or gene editing system to the cell.

Expression of natural or synthetic polynucleotides is typically achieved by operably linking a polynucleotide encoding the gene of interest, e.g., nucleotide sequence encoding the variant Cas12i3 polypeptide, to a promoter and incorporating the construct into an expression vector. The expression vector is not particularly limited as long as it includes a polynucleotide encoding the variant Cas12i3 polypeptide of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the variant Cas12i3 polypeptide from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding the variant Cas12i3 polypeptide of the present invention has been transferred into the host cells and then expressed without fail.

The preparation method for recombinant expression vectors is not particularly limited, and examples thereof include methods using a plasmid, a phage or a cosmid.

### Methods of Expression

The present invention includes a method for protein expression, comprising translating the variant Cas12i3 polypeptide described herein.

In some embodiments, a host cell described herein is used to express the variant Cas12i3 polypeptide. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the variant Cas12i3 polypeptide. After expression of the variant Cas12i3 polypeptide, the host cells can be collected and variant Cas12i3 polypeptide purified from the cultures etc. according to conventional methods (for example, filtration, centrifugation, cell disruption, gel filtration chromatography, ion exchange chromatography, etc.).

In some embodiments, the methods for variant Cas12i3 polypeptide expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the variant Cas12i3 polypeptide. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the variant Cas12i3 polypeptide.

A variety of methods can be used to determine the level of production of a mature variant Cas12i3 polypeptide in a host cell. Such methods include, but are not limited to, for example, methods that utilize either polyclonal or monoclonal antibodies specific for the variant Cas12i3 polypeptide or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the variant Cas12i3 polypeptide in a cell, comprising providing a polyribonucleotide encoding the variant Cas12i3 polypeptide to a host cell wherein the polyribonucleotide encodes the variant Cas12i3 polypeptide, expressing the variant Cas12i3 polypeptide in the cell, and obtaining the variant Cas12i3 polypeptide from the cell.

### Variant Binary Complexing

Generally, the variant Cas12i3 polypeptide and the RNA guide bind to each other in a molar ratio of about 1:1 to form the variant binary complex. The variant Cas12i3 polypeptide and the RNA guide, either alone or together, do not naturally occur.

In some embodiments, the variant Cas12i3 polypeptide can be overexpressed in a host cell and purified as described herein, then complexed with the RNA guide (e.g., in a test tube) to form a variant effector ribonucleoprotein (RNP) (e.g., variant binary complex).

In some embodiments, the variant binary complex exhibits increased binding affinity to a target nucleic acid, increased on-target binding activity, increased on-target binding specificity, increased ternary complex formation with a target nucleic acid, and/or increased stability over a range of incubation times. In some embodiments, the variant binary complex exhibits decreased off-target binding to a non-target nucleic acid and/or decreased dissociation from a target nucleic acid over a range of incubation times. In some embodiments, the variant binary complex exhibits increased target nucleic acid complex formation, target nucleic acid activity, and/or target nucleic acid specificity over a range of incubation times.

In some embodiments, complexation of a binary complex occurs at a temperature lower than about any one of 20° C., 21° C., 22° C., 23° C., 24° C., 25° C., 26° C., 27° C., 28° C. 29° C. 30° C. 31° C. 32° C., 33° C., 34° C., 35° C., 36° C. 37° C. 38° C., 39° C. 40° C. 41° C., 42° C., 43° C., 44° C., 45° C., 50° C., or 55° C. In some embodiments, the variant Cas12i3 polypeptide does not dissociate from the RNA guide or bind to a free RNA at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours. In some embodiments, after binary complex formation, the variant effector ribonucleoprotein complex does not exchange the RNA guide with a different RNA.

In some embodiments, the variant Cas12i3 polypeptide and RNA guide are complexed in a binary complexation buffer. In some embodiments, the variant Cas12i3 polypeptide is stored in a buffer that is replaced with a binary complexation buffer to form a complex with the RNA guide. In some embodiments, the variant Cas12i3 polypeptide is stored in a binary complexation buffer.

In some embodiments, the binary complexation buffer has a pH in a range of about 7.3 to 8.6. In one embodiment, the pH of the binary complexation buffer is about 7.3. In one embodiment, the pH of the binary complexation buffer is about 7.4. In one embodiment, the pH of the binary complexation buffer is about 7.5. In one embodiment, the pH of the binary complexation buffer is about 7.6. In one embodiment, the pH of the binary complexation buffer is about 7.7. In one embodiment, the pH of the binary complexation buffer is about 7.8. In one embodiment, the pH of the binary complexation buffer is about 7.9. In one embodiment, the pH of the binary complexation buffer is about 8.0. In one embodiment, the pH of the binary complexation buffer is about 8.1. In one embodiment, the pH of the binary complexation buffer is about 8.2. In one embodiment, the pH of the binary complexation buffer is about 8.3. In one embodiment, the pH of the binary complexation buffer is about 8.4. In one embodiment, the pH of the binary complexation buffer is about 8.5. In one embodiment, the pH of the binary complexation buffer is about 8.6.

The thermostability of the variant Cas12i3 polypeptide can increase under favorable conditions such as the addition of an RNA guide, e.g., binding an RNA guide.

In some embodiments, the variant Cas12i3 polypeptide can be overexpressed and complexed with the RNA guide in a host cell prior to purification as described herein. In some embodiments, mRNA or DNA encoding the variant Cas12i3 polypeptide is introduced into a cell so that the variant Cas12i3 polypeptide is expressed in the cell. The RNA guide, which guides the variant Cas12i3 polypeptide to the desired target nucleic acid is also introduced into the cell, whether simultaneously, separately or sequentially from a single mRNA or DNA construct, such that the necessary ribonucleoprotein complex is formed in the cell.

### Assessing Variant Binary Complex Stability and Functionality

Provided herein in certain embodiments are methods for identifying an optimal variant Cas12i3 polypeptide/RNA guide complex (referred to herein as the variant binary complex) including (a) combining a variant Cas12i3 polypeptide and an RNA guide in a sample to form the variant binary complex; (b) measuring a value of the variant binary complex; and (c) determining the variant binary complex is optimal over the reference molecule, if the value of the variant binary complex is greater than a value of a reference molecule. In some embodiments, the value may include, but is not limited to, a stability measurement (e.g., Tₘ value, thermostability), a rate of binary complex formation, RNA guide binding specificity, and/or complex activity.

In some embodiments, an optimal variant Cas12i3 polypeptide/RNA guide complex (i.e., a variant binary complex) is identified by the steps of: (a) combining a variant Cas12i3 polypeptide and an RNA guide in a sample to form the variant binary complex; (b) detecting a Tₘ value of the variant binary complex; and (c) determining the variant binary complex is stable if the Tₘ value of the variant binary complex is greater than a Tₘ value of a reference molecule or a Tₘ reference value by at least 8° C.

The methods involving a step of measuring the thermostability of a variant Cas12i3 polypeptide/RNA guide complex (i.e., a variant binary complex) may include, without limitation, methods of determining the stability of a variant binary complex, methods of determining a condition that promotes a stable variant binary complex, methods of screening for a stable variant binary complex, and methods for identifying an optimal gRNA to form a stable variant binary complex. In certain embodiments, a thermostability value of a variant binary complex may be measured.

Additionally, in certain embodiments, a thermostability value of a reference molecule may also be measured. In certain embodiments, a variant binary complex may be determined to be stable if the measured thermostability value of the variant binary complex is greater than the measured thermostability value of the reference molecule or a thermostability reference value, measured under the same experimental conditions, as described herein. In certain embodiments, the reference molecule may be the variant Cas12i3 polypeptide absent an RNA guide.

In certain embodiments, the thermostability value that is measured may be a denaturation temperature value. In these embodiments, the thermostability reference value is a denaturation temperature reference value. In certain embodiments, the thermostability value that is measured may be a Tₘ value. In these embodiments, the thermostability reference value may be a Tₘ reference value. In certain embodiments, the thermostability value may be measured using a thermal shift assay. In certain embodiments, an assay used to measure thermostability may involve a technique described herein including, but not limited to, thermal denaturation assays, thermal shift assays, differential scanning calorimetry (DSC), differential scanning fluorimetry (DSF), isothermal titration calorimetry (ITC), pulse-chase methods, bleach-chase methods, cycloheximide-chase methods, circular dichroism (CD) spectroscopy, crystallization, and fluorescence-based activity assays.

In certain embodiments, a variant binary complex may be identified if the rate of variant Cas12i3 polypeptide/RNA guide complex formation, RNA guide binding specificity, and/or complex activity of the variant binary complex is greater than a value of the reference molecule or the reference value (e.g., a value of a parent polypeptide/RNA guide complex, referred to herein as a parent binary complex). For example, in certain embodiments, the variant binary complex may be identified if the value of a rate of variant Cas12i3 polypeptide/RNA guide complex formation, RNA guide binding specificity, and/or complex activity of the variant binary complex is at least X % greater than a value of the reference molecule or the reference value (e.g., a value of a parent binary complex). In certain embodiments, the methods described herein may further comprise steps that include measuring the activity of the variant binary complex as described herein.

### Variant Ternary Complexing

In some embodiments, the variant Cas12i3 polypeptide, RNA guide, and target nucleic acid, as described herein, form a variant ternary complex (e.g., in a test tube or cell). Generally, the variant Cas12i3 polypeptide, the RNA guide, and the target nucleic acid associate with each other in a molar ratio of about 1:1:1 to form the variant ternary complex. The variant Cas12i3 polypeptide, the RNA guide, and the target nucleic acid, either alone or together, do not naturally occur.

In some embodiments, the variant binary complex (e.g., complex of variant Cas12i3 polypeptide and RNA guide) as described herein, is further complexed with the target nucleic acid (e.g., in a test tube or cell) to form a variant ternary complex.

In some embodiments, complexation of the ternary complex occurs at a temperature lower than about any one of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 50° C., or 55° C. In some embodiments, the variant binary complex does not dissociate from the target nucleic acid or bind to a free nucleic acid (e.g., free DNA) at about 37° C. over an incubation period of at least about any one of 10 mins, 15 mins, 20 mins, 25 mins, 30 mins, 35 mins, 40 mins, 45 mins, 50 mins, 55 mins, 1 hr, 2 hr, 3 hr, 4 hr, or more hours. In some embodiments, after ternary complex formation, a variant binary complex does not exchange the target nucleic acid with a different nucleic acid.

In some embodiments, the variant Cas12i3 polypeptide, RNA guide, and target nucleic acid are complexed in a ternary complexation buffer. In some embodiments, the variant Cas12i3 polypeptide is stored in a buffer that is replaced with a ternary complexation buffer to form a complex with the RNA guide and target nucleic acid. In some embodiments, the variant Cas12i3 polypeptide is stored in a ternary complexation buffer.

In some embodiments, the variant binary complex and target nucleic acid are complexed in a ternary complexation buffer. In some embodiments, the variant binary complex is stored in a buffer that is replaced with a ternary complexation buffer to form a complex with the target nucleic acid. In some embodiments, the variant binary complex is stored in a ternary complexation buffer.

In some embodiments, the ternary complexation buffer has a pH in a range of about 7.3 to 8.6. In one embodiment, the pH of the ternary complexation buffer is about 7.3. In one embodiment, the pH of the ternary complexation buffer is about 7.4. In one embodiment, the pH of the ternary complexation buffer is about 7.5. In one embodiment, the pH of the ternary complexation buffer is about 7.6. In one embodiment, the pH of the ternary complexation buffer is about 7.7. In one embodiment, the pH of the ternary complexation buffer is about 7.8. In one embodiment, the pH of the ternary complexation buffer is about 7.9. In one embodiment, the pH of the ternary complexation buffer is about 8.0. In one embodiment, the pH of the ternary complexation buffer is about 8.1. In one embodiment, the pH of the ternary complexation buffer is about 8.2. In one embodiment, the pH of the ternary complexation buffer is about 8.3. In one embodiment, the pH of the ternary complexation buffer is about 8.4. In one embodiment, the pH of the ternary complexation buffer is about 8.5. In one embodiment, the pH of the ternary complexation buffer is about 8.6.

The thermostability of a variant Cas12i3 polypeptide can increase under favorable conditions such as the addition of an RNA guide and target nucleic acid.

### Assessing Variant Ternary Complex Stability and Functionality

Provided herein in certain embodiments are methods for identifying an optimal variant ternary complex including (a) combining a variant Cas12i3 polypeptide, an RNA guide, and a target nucleic acid in a sample to form the variant ternary complex; (b) measuring a value of the variant ternary complex; and (c) determining the variant ternary complex is optimal over the reference molecule, if the value of the variant ternary complex is greater than a value of a reference molecule. In some embodiments, the value may include, but is not limited to, a stability measurement (e.g., Tₘ value, thermostability), a rate of ternary complex formation, a DNA binding affinity measurement, a DNA binding specificity measurement, and/or a complex activity measurement (e.g., nuclease activity measurement).

In some embodiments, an optimal variant ternary complex is identified by the steps of: (a) combining a variant Cas12i3 polypeptide, an RNA guide, and a target nucleic acid in a sample to form the variant ternary complex; (b) detecting a Tₘ value of the variant ternary complex; and (c) determining the variant ternary complex is stable if the Tₘ value of the variant ternary complex is greater than a Tₘ value of a reference molecule or a Tₘ reference value by at least 8° C.

The methods involving a step of measuring the thermostability of a variant ternary complex may include, without limitation, methods of determining the stability of a variant ternary complex, methods of determining a condition that promotes a stable variant ternary complex, methods of screening for a stable variant ternary complex, and methods for identifying an optimal binary complex to form a stable variant ternary complex. In certain embodiments, a thermostability value of a variant ternary complex may be measured.

Additionally, in certain embodiments, a thermostability value of a reference molecule may also be measured. In certain embodiments, a variant ternary complex may be determined to be stable if the measured thermostability value of the variant ternary complex is greater than the measured thermostability value of the reference molecule or a thermostability reference value, measured under the same experimental conditions, as described herein. In certain embodiments, the reference molecule may be the variant Cas12i3 polypeptide absent an RNA guide and/or target nucleic acid.

In certain embodiments, the thermostability value that is measured may be a denaturation temperature value. In these embodiments, the thermostability reference value is a denaturation temperature reference value. In certain embodiments, the thermostability value that is measured may be a Tₘ value. In these embodiments, the thermostability reference value may be a Tₘ reference value. In certain embodiments, the thermostability value may be measured using a thermal shift assay. In certain embodiments, an assay used to measure thermostability may involve a technique described herein including, but not limited to, differential scanning fluorimetry (DSF), differential scanning calorimetry (DSC), or isothermal titration calorimetry (ITC).

In certain embodiments, a variant ternary complex may be identified if the rate of ternary complex formation, DNA binding affinity, DNA binding specificity, and/or complex activity (e.g., nuclease activity) of the variant ternary complex is greater than a value of the reference molecule or the reference value (e.g., a value of a parent ternary complex). For example, in certain embodiments, the variant ternary complex may be identified if the value of a rate of ternary complex formation. DNA binding affinity, DNA binding specificity, and/or complex activity of the variant ternary complex is at least X % greater than a value of the reference molecule or the reference value (e.g., a value of a parent ternary complex). In certain embodiments, the methods described herein may further comprise steps that include measuring the activity of the variant ternary complex as described herein.

### Delivery

Compositions or gene editing systems, or complexes described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the variant Cas12i3 polypeptide, RNA guide, donor DNA, etc.), one or more transcripts thereof, and/or a pre-formed variant Cas12i3 polypeptide/RNA guide complex (i.e., variant binary complex) to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, the present application further provides cells produced by such methods, and organisms (such as animals, plants, or fungi) comprising or produced from such cells.

### Cells

Polypeptides, compositions or gene editing systems, or complexes described herein may be delivered to a variety of cells. In some embodiments, the cell is an isolated cell. In some embodiments the cell is in cell culture. In some embodiments, the cell is ex vivo. In some embodiments, the cell is obtained from a living organism, and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the bacterial cell is not related to the bacterial species from which the parent polypeptide is derived. In some embodiments, the cell is an archaeal cell or derived from an archaeal cell. In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, and transgenic varieties thereof. Cell lines are available from a variety of sources known to those with skill in the art (see, e.g., the American Type Culture Collection (ATCC) (Manassas, Va.)). In some embodiments, a cell transfected with one or more nucleic acids (such as Ago-coding vector and gDNA) or Ago-gDNA complex described herein is used to establish a new cell line comprising one or more vector-derived sequences to establish a new cell line comprising modification to the target nucleic acid. In some embodiments, cells transiently or non-transiently transfected with one or more nucleic acids (such as variant Cas12i3 polypeptide-encoding vector and RNA guide) or variant Cas12i3 polypeptide/RNA guide complex (i.e., variant binary complex) described herein, or cell lines derived from such cells are used in assessing one or more test compounds.

In some embodiments, the method comprises introducing into a host cell one or more nucleic acids comprising nucleotide sequences encoding a DNA-targeting RNA (e.g., RNA guide) and/or the variant Cas12i3 polypeptide. In one embodiment, a cell comprising a target DNA is in vitro, in vivo, or ex vivo. In other embodiments, nucleic acids comprising nucleotide sequences encoding a DNA-targeting RNA (e.g., RNA guide) and/or the variant Cas12i3 polypeptide include recombinant expression vectors e.g., including but not limited to adeno-associated virus constructs, recombinant adenoviral constructs, recombinant lentiviral constructs, recombinant retroviral constructs, and the like.

In some embodiments, the cell is a primary cell. For example, cultures of primary cells can be passaged 0 times, 1 time, 2 times, 4 times, 5 times, 10 times, 15 times or more. In some embodiments, the primary cells are harvest from an individual by any known method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

In some embodiments, the variant Cas12i3 polypeptide has nuclease activity that induces double-stranded breaks or single-stranded breaks in a target nucleic acid, (e.g. genomic DNA). The double-stranded break can stimulate cellular endogenous DNA-repair pathways, including Homology Directed Recombination (HDR), Non-Homologous End Joining (NHEJ), or Alternative Non-Homologues End-Joining (A-NHEJ). NHEJ can repair cleaved target nucleic acid without the need for a homologous template. This can result in deletion or insertion of one or more nucleotides into the target nucleic acid. HDR can occur with a homologous template, such as the donor DNA. The homologous template can comprise sequences that are homologous to sequences flanking the target nucleic acid cleavage site. In some cases, HDR can insert an exogenous polynucleotide sequence into the cleaved target nucleic acid. The modifications of the target DNA due to NHEJ and/or HDR can lead to, for example, mutations, deletions, alterations, integrations, gene correction, gene replacement, gene tagging, transgene knock-in, gene disruption, and/or gene knock-outs.

In some embodiments, the cell culture is synchronized to enhance the efficiency of the methods. In some embodiments, cells in S and G2 phases are used for HDR-mediated gene editing. In some embodiments, the cell can be subjected to the method at any cell cycle. In some embodiments, cell over-plating significantly reduces the efficacy of the method. In some embodiments, the method is applied to a cell culture at no more than about any one of 40%, 45%, 50%, 55%, 60%, 65%, or 70% confluency.

In some embodiments, binding of the variant Cas12i3 polypeptide/RNA guide complex (i.e., variant binary complex) to the target nucleic acid in the cell recruits one or more endogenous cellular molecules or pathways other than DNA repair pathways to modify the target nucleic acid. In some embodiments, binding of the variant binary complex blocks access of one or more endogenous cellular molecules or pathways to the target nucleic acid, thereby modifying the target nucleic acid. For example, binding of the variant binary complex may block endogenous transcription or translation machinery to decrease the expression of the target nucleic acid.

In some embodiments, a method for modifying a target DNA molecule in a cell is provided. The method comprises contacting the target DNA molecule inside of a cell with a variant Cas12i3 polypeptide described herein; and a single molecule DNA-targeting RNA comprising, in 5′ to 3′ order, a first nucleotide segment that hybridizes with a target sequence of the target DNA molecule; a nucleotide linker; and a second nucleotide segment that hybridizes with the first nucleotide segment to form a double-stranded RNA duplex. The variant Cas12i3 polypeptide forms a complex with the single molecule DNA-targeting RNA inside the cell and the target DNA molecule is modified.

### Kits

The invention also provides kits that can be used, for example, to carry out a method described herein. In some embodiments, the kits include a variant Cas12i3 polypeptide of the invention, e.g., a variant of Table 2, 3, 4, or 6. In some embodiments, the kits include a polynucleotide that encodes such a variant Cas12i3 polypeptide, and optionally the polynucleotide is comprised within a vector, e.g., as described herein. The kits also can optionally include an RNA guide, e.g., as described herein. The RNA guide of the kits of the invention can be designed to target a sequence of interest, as is known in the art. The effector variant and the RNA guide can be packaged within the same vial or other vessel within a kit or can be packaged in separate vials or other vessels, the contents of which can be mixed prior to use. The kits can additionally include, optionally, a buffer and/or instructions for use of the effector variant and/or RNA guide.

All references and publications cited herein are hereby incorporated by reference.

### ADDITIONAL EMBODIMENTS

Provided below are additional embodiments, which are also within the scope of the present disclosure.

Embodiment 1: A composition comprising a variant Cas12i3 polypeptide or a complex comprising the variant Cas12i3 polypeptide, wherein the variant Cas12i3 polypeptide comprises one or more substitutions of Table 2, Table 3, Table 4, or Table 6 and wherein the variant Cas12i3 polypeptide or the complex exhibits enhanced enzymatic activity, enhanced binding activity, enhanced binding specificity, and/or enhanced stability relative to a parent polypeptide or a complex comprising the parent polypeptide. In some embodiments, the one or more substitutions is relative to the sequence of SEQ ID NO: 3.

In any of the compositions of Embodiment 1, the variant Cas12i3 polypeptide may further comprise a substitution of Table 2.

In any of the compositions of Embodiment 1, the variant Cas12i3 polypeptide may comprise an amino acid sequence having at least 95% identity to SEQ ID NO: 7.

In any of the compositions of Embodiment 1, the variant Cas12i3 polypeptide may comprise the amino acid sequence set forth in SEQ ID NO: 7.

In any of the compositions of Embodiment 1, the variant Cas12i3 polypeptide may comprise a substitution or a set of substitutions selected from the group consisting of: S536R, S559R, R565G, H566R, S571R, K583R, K586R, L592R, M595R, E596R, I630R, T631R, N646R, L703R, S704R, N711R, C724R, S738R, S801R, T844R, T977R, and T1040R.

In any of the compositions of Embodiment 1, the variant Cas12i3 polypeptide may comprise a set of mutations selected from the group consisting of: (a) mutations at positions S536, N711, and S801, which optionally are amino acid substitutions of S536R, N711R. and S801R; (b) mutations at positions S536, N711. C724, and S801, which optionally are amino acid substitutions of S536R, N711R, C724R, and S801R; (c) mutations at positions S536, N711, and C724, which optionally are amino acid substitutions of S536R, N711R, and C724R; (d) mutations at positions S536 and N711, which optionally are amino acid substitutions of S536R and N711R; (e) mutations at positions S536, K583, N711, and S801, which optionally are amino acid substitutions of S536R, K583R, N711R, and S801R; (f) mutations at positions S536, K583, N711, and C724, which optionally are amino acid substitutions of S536R, K583R, N711R, and C724R; (g) mutations at positions S536, K583, C724, and S801, which optionally are amino acid substitutions of S536R, K583R, C724R, and S801R; (h) mutations at positions S536, K583, and C724, which optionally are amino acid substitutions of S536R, K583R, and C724R; (i) mutations at positions S536 and K583, which optionally are amino acid substitutions of S536R and K583R; (j) mutations at positions S536, C724, and S801, which optionally are amino acid substitutions of S536R, C724R, and S801R; (k) mutations at positions S536 and C724, which optionally are amino acid substitutions of S536R and C724R; (l) mutations at positions N711 and S801, which optionally are amino acid substitutions of N711R and S801R; (m) mutations at positions N711, C724, and S801, which optionally are amino acid substitutions of N711R, C724R, and S801R; (n) mutations at positions N711 and C724, which optionally are amino acid substitutions of N711R and C724R; (o) mutations at positions K583 and S801, which optionally are amino acid substitutions of K583R and S801R; (p) mutations at positions K583, N711, and S801, which optionally are amino acid substitutions of K583R, N711R, and S801R; (q) mutations at positions K583, N711, C724, and S801, which optionally are amino acid substitutions of K583R, N711R, C724R, and S801R; (r) mutations at positions K583, N711, and C724, which optionally are amino acid substitutions of K583R, N711R, and C724R; (s) mutations at positions K583 and N711, which optionally are amino acid substitutions of K583R and N711R; (t) mutations at positions K583, C724, and S801, which optionally are amino acid substitutions of K583R, C724R, and S801R; (u) mutations at positions K583 and C724, which optionally are amino acid substitutions of K583R and C724R; and (v) mutations at positions C724 and S801, which optionally are amino acid substitutions of C724R and S801R.

In any of the compositions of Embodiment 1, the enhanced enzymatic activity may be enhanced nuclease activity.

In any of the compositions of Embodiment 1, the variant Cas12i3 polypeptide may exhibit enhanced binding activity to an RNA guide relative to the parent polypeptide.

In any of the compositions of Embodiment 1, the variant Cas12i3 polypeptide may exhibit enhanced binding specificity to an RNA guide relative to the parent polypeptide.

In any of the compositions of Embodiment 1, the complex comprising the variant Cas12i3 polypeptide may be a variant binary complex that further comprises an RNA guide, and the variant binary complex exhibits enhanced binding activity to a target nucleic acid (e.g., on-target binding activity) relative to a parent binary complex.

In any of the compositions of Embodiment 1, the complex comprising the variant Cas12i3 polypeptide may be a variant binary complex that further comprises an RNA guide, and the variant binary complex exhibits enhanced binding specificity to a target nucleic acid (e.g., on-target binding specificity) relative to a parent binary complex.

In any of the compositions of Embodiment 1, the complex comprising the variant Cas12i3 polypeptide may be a variant binary complex that further comprises an RNA guide, and the variant binary complex exhibits enhanced stability relative to a parent binary complex.

In any of the compositions of Embodiment 1, the variant binary complex and a target nucleic acid may form a variant ternary complex, and the variant ternary complex exhibits increased stability relative to a parent ternary complex.

In any of the compositions of Embodiment 1, the variant Cas12i3 polypeptide may further exhibit enhanced binary complex formation, enhanced protein-RNA interactions, and/or decreased dissociation from an RNA guide relative to the parent polypeptide.

In any of the compositions of Embodiment 1, the variant binary complex may further exhibit decreased dissociation from the target nucleic acid, and/or decreased off-target binding to a non-target nucleic acid relative to the parent binary complex.

In any of the compositions of Embodiment 1, the enhanced enzymatic activity, enhanced binding activity, enhanced binding specificity, and/or enhanced stability may occur over a range of temperatures, e.g., 20° C. to 65° C.

In any of the compositions of Embodiment 1, the enhanced enzymatic activity, enhanced binding activity, enhanced binding specificity, and/or enhanced stability may occur over a range of incubation times. In any of the compositions of Embodiment 1, the enhanced enzymatic activity, enhanced binding activity, enhanced binding specificity, and/or enhanced stability may occur in a buffer having a pH in a range of about 7.3 to about 8.6.

In any of the compositions of Embodiment 1, the enhanced enzymatic activity, enhanced binding activity, enhanced binding specificity, and/or enhanced stability may occur when a Tₘ value of the variant Cas12i3 polypeptide, variant binary complex, or variant ternary complex may be at least 8° C. greater than the Tₘ value of the parent polypeptide, parent binary complex, or parent ternary complex.

In any of the compositions of Embodiment 1, the variant Cas12i3 polypeptide may comprise a RuvC domain or a split RuvC domain.

In any of the compositions of Embodiment 1, the parent polypeptide may comprise the sequence of SEQ ID NO: 3.

In any of the compositions of Embodiment 1, the RNA guide may comprise a direct repeat sequence and a spacer sequence.

In any of the compositions of Embodiment 1, the direct repeat may comprise: a. nucleotide 1 through nucleotide 36 of a sequence that is at least 90% identical to SEQ ID NO: 4 or SEQ ID NO: 5; b. nucleotide 2 through nucleotide 36 of a sequence that is at least 90% identical to SEQ ID NO: 4 or SEQ ID NO: 5; c. nucleotide 3 through nucleotide 36 of a sequence that is at least 90% identical to SEQ ID NO: 4 or SEQ ID NO: 5; d. nucleotide 4 through nucleotide 36 of a sequence that is at least 90% identical to SEQ ID NO: 4 or SEQ ID NO: 5; e. nucleotide 5 through nucleotide 36 of a sequence that is at least 90% identical to SEQ ID NO: 4 or SEQ ID NO: 5; f. nucleotide 6 through nucleotide 36 of a sequence that is at least 90% identical to SEQ ID NO: 4 or SEQ ID NO: 5; g. nucleotide 7 through nucleotide 36 of a sequence that is at least 90% identical to SEQ ID NO: 4 or SEQ ID NO: 5; h. nucleotide 8 through nucleotide 36 of a sequence that is at least 90% identical to SEQ ID NO: 4 or SEQ ID NO: 5; i. nucleotide 9 through nucleotide 36 of a sequence that is at least 90% identical to SEQ ID NO: 4 or SEQ ID NO: 5; j. nucleotide 10 through nucleotide 36 of a sequence that is at least 90% identical to SEQ ID NO: 4 or SEQ ID NO: 5; k. nucleotide 11 through nucleotide 36 of a sequence that is at least 90% identical to SEQ ID NO: 4 or SEQ ID NO: 5; l. nucleotide 12 through nucleotide 36 of a sequence that is at least 90% identical to SEQ ID NO: 4 or SEQ ID NO: 5; m. nucleotide 13 through nucleotide 36 of a sequence that is at least 90% identical to SEQ ID NO: 4 or SEQ ID NO: 5; n. nucleotide 14 through nucleotide 36 of a sequence that is at least 90% identical to SEQ ID NO: 4 or SEQ ID NO: 5; or o. a sequence that is at least 90% identical to a sequence of SEQ ID NO: 6 or a portion thereof.

In any of the compositions of Embodiment 1, the direct repeat may comprise: a. nucleotide 1 through nucleotide 36 of a sequence that is at least 95% identical to SEQ ID NO: 4 or SEQ ID NO: 5; b. nucleotide 2 through nucleotide 36 of a sequence that is at least 95% identical to SEQ ID NO: 4 or SEQ ID NO: 5; c. nucleotide 3 through nucleotide 36 of a sequence that is at least 95% identical to SEQ ID NO: 4 or SEQ ID NO: 5; d. nucleotide 4 through nucleotide 36 of a sequence that is at least 95% identical to SEQ ID NO: 4 or SEQ ID NO: 5; e. nucleotide 5 through nucleotide 36 of a sequence that is at least 95% identical to SEQ ID NO: 4 or SEQ ID NO: 5; f. nucleotide 6 through nucleotide 36 of a sequence that is at least 95% identical to SEQ ID NO: 4 or SEQ ID NO: 5; g. nucleotide 7 through nucleotide 36 of a sequence that is at least 95% identical to SEQ ID NO: 4 or SEQ ID NO: 5; h. nucleotide 8 through nucleotide 36 of a sequence that is at least 95% identical to SEQ ID NO: 4 or SEQ ID NO: 5; i. nucleotide 9 through nucleotide 36 of a sequence that is at least 95% identical to SEQ ID NO: 4 or SEQ ID NO: 5; j. nucleotide 10 through nucleotide 36 of a sequence that is at least 95% identical to SEQ ID NO: 4 or SEQ ID NO: 5; k. nucleotide 11 through nucleotide 36 of a sequence that is at least 95% identical to SEQ ID NO: 4 or SEQ ID NO: 5; l. nucleotide 12 through nucleotide 36 of a sequence that is at least 95% identical to SEQ ID NO: 4 or SEQ ID NO: 5; m. nucleotide 13 through nucleotide 36 of a sequence that is at least 95% identical to SEQ ID NO: 4 or SEQ ID NO: 5; n. nucleotide 14 through nucleotide 36 of a sequence that is at least 95% identical to SEQ ID NO: 4 or SEQ ID NO: 5; or o. a sequence that is at least 95% identical to a sequence of SEQ ID NO: 6 or a portion thereof.

In any of the compositions of Embodiment 1, the direct repeat may comprise: a. nucleotide 1 through nucleotide 36 of SEQ ID NO: 4 or SEQ ID NO: 5; b. nucleotide 2 through nucleotide 36 of SEQ ID NO: 4 or SEQ ID NO: 5; c. nucleotide 3 through nucleotide 36 of SEQ ID NO: 4 or SEQ ID NO: 5; d. nucleotide 4 through nucleotide 36 of SEQ ID NO: 4 or SEQ ID NO: 5; e. nucleotide 5 through nucleotide 36 of SEQ ID NO: 4 or SEQ ID NO: 5; f. nucleotide 6 through nucleotide 36 of SEQ ID NO: 4 or SEQ ID NO: 5; g. nucleotide 7 through nucleotide 36 of SEQ ID NO: 4 or SEQ ID NO: 5; h. nucleotide 8 through nucleotide 36 of SEQ ID NO: 4 or SEQ ID NO: 5; i. nucleotide 9 through nucleotide 36 of SEQ ID NO: 4 or SEQ ID NO: 5; j. nucleotide 10 through nucleotide 36 of SEQ ID NO: 4 or SEQ ID NO: 5; k. nucleotide 11 through nucleotide 36 of SEQ ID NO: 4 or SEQ ID NO: 5; l. nucleotide 12 through nucleotide 36 of SEQ ID NO: 4 or SEQ ID NO: 5; m. nucleotide 13 through nucleotide 36 of SEQ ID NO: 4 or SEQ ID NO: 5; n. nucleotide 14 through nucleotide 36 of SEQ ID NO: 4 or SEQ ID NO: 5; or o. SEQ ID NO: 6 or a portion thereof.

In any of the compositions of Embodiment 1, the spacer sequence may comprise between 15 and 35 nucleotides in length.

In any of the compositions of Embodiment 1, the target nucleic acid may comprise a sequence complementary to a nucleotide sequence in the spacer sequence.

In any of the compositions of Embodiment 1, the target nucleic acid may be adjacent to a protospacer adjacent motif (PAM) sequence (and is herein referred to as a “target sequence”), wherein the PAM sequence comprises a nucleotide sequence set forth as 5′-TTN-3′ or 5′-NTTN-3′, wherein N is any nucleotide.

In any of the compositions of Embodiment 1, the target nucleic acid may be single-stranded DNA or double-stranded DNA.

In any of the compositions of Embodiment 1, the variant Cas12i3 polypeptide may further comprise a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

Embodiment 2: A composition comprising a nucleic acid that encodes a Cas12i3 polypeptide described herein, wherein optionally the nucleic acid is codon-optimized for expression in a cell.

In any of the compositions of Embodiment 2, the nucleic acid encoding the variant Cas12i3 polypeptide may be operably linked to a promoter.

In any of the compositions of Embodiment 2, the nucleic acid encoding the variant Cas12i3 polypeptide may be in a vector.

In any of the compositions of Embodiment 2, the vector may comprise a retroviral vector, a lentiviral vector, a phage vector, an adenoviral vector, an adeno-associated vector, or a herpes simplex vector.

In Embodiment 2, the composition may be present in a delivery composition comprising a nanoparticle, a liposome, an exosome, a microvesicle, or a gene-gun.

Embodiment 3: A cell comprising the composition described herein.

In any of the cells of Embodiment 3, the cell may be a eukaryotic cell or a prokaryotic cell.

In any of the cells of Embodiment 3, the cell may be a mammalian cell or a plant cell.

In any of the cells of Embodiment 3, the cell may be a human cell.

Embodiment 4: A method of producing a composition described herein.

Embodiment 5: A method of delivering a composition described herein.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Engineering of Variant Constructs

In this Example, variant constructs were generated.

DNA templates comprising single mutations were constructed via two PCR steps using mutagenic forward and mutagenic reverse primers ordered from IDT. In the first step, two sets of PCR reactions were conducted in 384 plates to generate two fragments. The overlapping regions of two PCR fragments contained the desired single mutations and allowed the assembly of the entire DNA template via a second PCR. In the second step, the purified fragments from the first step were used as the template for the overlapping PCR (OL PCR) and the Fw and Rv oligos annealing to the vector backbone as the OL PCR primers. The resulting linear DNA templates contained a T7 promoter, a T7 terminator, and the open-reading frame for the effector.

These linear DNA templates were used directly in a cell-free transcription and translation system to express the effector variants containing the single mutations. The variant constructs were further individually transferred into transient transfection vectors. Additionally, DNA templates comprising combinatorial mutations were prepared by PCR and subsequently transferred into transient transfection vectors.

### Example 2—Florescence Polarization Assay for Variant Binary Complex Detection

In this Example, the ability of an effector polypeptide and an RNA guide to form a binary complex is assessed through a fluorescence polarization assay.

Linear ssDNA fragments comprising the reverse complement of the T7 RNA polymerase promoter sequence upstream of the direct repeat sequence and desired 20 bp RNA guide target are synthesized by IDT. Linear dsDNA in vitro transcription (IVT) templates are then generated by annealing a universal T7 forward oligo (95-4° C. at 5° C./minute) to the reverse complement ssDNA and filled in with Klenow fragment (New England Biolabs) for 15 minutes at 25° C. The resulting IVT template is then transcribed into an RNA guide using the HiScribe T7 High Yield RNA Synthesis Kit (New England Biolabs) at 37° C. for 4 hours. Following transcription, each RNA guide is purified using an RNA Clean and Concentrator Kit (Zymo) and stored at −20° C. until use.

The RNA guide is then labeled with 6-carboxyfluorescein (6-FAM) (IDT). 25 nM effector polypeptide (wild-type or variant Cas12i3 polypeptide) in 1× assay buffer (20 mM Tris-HCl (pH 7.5). 150 mM KCl, 5 mM MgCl₂, 1 mM DTT) is titrated with increasing concentrations of labeled RNA guide (7.5-250 nM). Complexes are incubated at 37° C. for 30 minutes before taking fluorescence polarization measurements using a microplate reader (Infinite 200 Pro. Tecan).

Binary complex formation at different temperatures is also investigated. Further binding experiments as described above are performed isothermally at 25, 50, 60, and 70° C.

Formation of a binary complex upon titration of an effector polypeptide (wild-type or variant Cas12i3 polypeptide) with increasing concentrations of RNA guide (or formation of a binary complex upon titration of RNA guide with increasing concentrations of an effector polypeptide) results in changes in fluorescence polarization signal, in millipolarization (mP) units. A binding curve is generated by plotting changes in fluorescence polarization signal over a range of RNA guide concentrations.

This Example indicates how binding affinities of effector polypeptides (wild-type or variant Cas12i3 polypeptide) to RNA guides can be determined and compared.

### Example 3—RNA Electrophoretic Mobility Shift Assay for Variant Binary Complex Detection

This Example describes use of an RNA EMSA to determine the ability of an effector polypeptide (wild-type or variant) to bind to an RNA guide.

Synthetic RNA guides from IDT are labeled with a 5′ IR800 dye using 5′ EndTag Labeling Kit (Vector Labs) and IRDye 800CW Maleimide (LI-COR Biosciences), as previously detailed in Yan et al., 2018. After labeling, the RNA guides are cleaned and concentrated via phenol chloroform extraction. Concentrations are quantified by Nanodrop.

For RNA binding assays, effector polypeptides (wild-type or variant Cas12i3 polypeptides) are diluted to 2.5 μM in 1× binding buffer (50 mM NaCl, 10 mM Tris-HCl, 10 mM MgCl₂, 1 mM DTT, pH 7.9. Polypeptides are then serially diluted from 2.5 μM to 37.5 μM in 1× binding buffer. The polypeptides are again diluted 1:10 in 1× binding buffer plus 50 nM IR800 labeled RNA guide and mixed thoroughly. These reactions can further include 0.5-5 μg tRNA, which serves as a competitive inhibitor to decrease nonspecific binding of polypeptide to RNA and thereby facilitate accurate specific binding determinations. Reactions are incubated at 37° C. for 1 hour. 1 μL 100× bromophenol blue is added to the reactions for dye front visualization, then the entire reaction is loaded onto a 6% DNA Retardation Gel (Thermofisher), which runs for 90 minutes at 80V. The gel is imaged on the Licor Odyssey CLx.

This assay relies on the principle that the rate at which RNA migrates through the gel is determined by its size. An RNA only sample is able to migrate a particular distance. However, if the RNA binds to a polypeptide, a band that represents a larger, less mobile RNA complex appears, which is “upshifted” on the gel.

Therefore, the intensities of two bands are measured: 1) an RNA only band and 2) a polypeptide-bound “upshifted” RNA band. If all RNA is bound to a polypeptide, only an upshifted band is observed. As the concentration of polypeptide decreases, the intensity of the upshifted band decreases, while the intensity of the RNA only band increases. In comparing RNA binding affinities for effector polypeptides (wild-type or variant Cas12i3 polypeptides), a higher polypeptide/RNA affinity is characterized by more specific binding at lower concentrations of polypeptide.

This Example indicates how binding affinities of wild-type effector polypeptides to RNA guides and binding affinities of variant Cas12i3 polypeptides to RNA guides can be determined and compared.

### Example 4—In Vitro Cleavage Assay for Variant Binary Complexes

This Example describes methods for preparing effector RNPs and for determining in vitro biochemical activity of effector (wild-type or variant) RNPs.

Effector vectors are transformed into E. coli BL21 (DE3) (New England BioLabs) and expressed under a T7 promoter. Transformed cells are initially grown overnight in 5 mL Luria Broth (Teknova)+50 μg/mL kanamycin, followed by inoculation into 1 L Terrific Broth media (Teknova)+50 μg/mL kanamycin. Cells are grown at 37° C. until an OD₆₀₀ of 0.6-0.8, then protein expression is induced with 0.5 mM IPTG. Cultures are then grown at 18° C. for an additional 14-18 hours. Cultures are harvested and pelleted via centrifugation, then resuspended in 1 mL extraction buffer per 5 g cell pellet (50 mM HEPES. pH 7.5, 500 mM NaCl. 5% glycerol. 0.5 mM TCEP). Cells are lysed via cell disruptor (Constant System Limited), then centrifuged at 20,000×g for 20 minutes at 4° C. in order to clarify the lysate. 0.2% polyethylenimine (PEI) is added to the clarified lysate and incubated at 4° C. with constant end-over-end rotation for 20 minutes. The lysate is then centrifuged again at 20.000×g for 10 minutes. The lysate is purified via ion exchange chromatography. After purification, fractions are run on SDS-PAGE gels, and fractions containing protein of the appropriate size are pooled and concentrated using 30 kD Amicon Ultra15 Centrifugal Units. Proteins are buffer exchanged into 12.5 mM HEPES PH 7.0, 120 mM NaCl. 0.5 mM TCEP, and 50% glycerol. Concentrations are then measured using the Nanodrop (Thermofisher), and proteins are stored at −20° C.

RNPs are prepared using a 2:1 ratio of synthetic crRNA (Integrated DNA Technologies) to protein. The RNPs are complexed for 30 minutes at 37° C. in 1×NEB2 buffer (50 mM NaCl, 10 mM Tris-HCl, 10 mM MgCl₂, 1 mM DTT, pH 7.9). After complexing, the RNPs are diluted using 1×NEB2 as a dilution buffer. Apo reactions (protein without RNA guide) are prepared in the same manner, making up the volume of crRNA with H₂O.

A target dsDNA substrate (Integrated DNA Technologies) is added at 20 nM to the RNP and apo samples. Reactions are mixed thoroughly then incubated at 37° C. for 1 hour, then quenched with 1 μL 20 mg/mL Proteinase K (Thermofisher). Reactions are incubated for another 15 minutes at 50° C., then the entire reaction is run on a 2% agarose E-gel (Thermofisher). Gels are visualized by ethidium bromide on a Gel Doc EZ Gel Imager (BioRad).

The intensities of two types of bands are measured: 1) a full-length (uncleaved) DNA band and 2) one or more downshifted cleaved DNA bands. An inactive RNP is characterized by a full-length DNA band. An active RNP yields one or more downshifted cleaved DNA bands. As the concentration of an active RNP decreases, the intensity of the full-length band increases, and the intensity of the cleaved band(s) decreases. In comparing activity of multiple RNPs, an RNP having higher activity than another is characterized by more intense cleaved bands at lower RNP concentrations.

The method of this Example allows for the comparison of in vitro cleavage activity of wild-type or variant effector RNPs (binary complexes) on target DNA.

### Example 5—In Vitro Stability Assays of Variant Cas12i3 Polypeptides and Variant Binary Complexes

In this Example, the stability of a variant RNP is assessed.

For the accelerated stability study, RNPs (5 μM) are generated in the same manner as described in Example 4, and the samples are subsequently stored at 25° C. for 48 hours.

In vitro cleavage assays (as described in Example 4) are performed on the RNP samples. These results are compared with those of Example 4 to determine the extent to which variant RNPs stored at 25° C. for 48 hours retain biochemical activity.

Apo polypeptide (without RNA guide) is also incubated at 25° C. for 48 hours. RNA EMSA assays are performed on the apo samples using the method described in Example 3. These results are compared with those of Example 3 to determine the extent to which a variant effector is able to form a binary complex with an RNA guide.

Apo samples incubated at 25° C. for 48 hours are also complexed with RNA guides to form RNPs, using the method described in Example 4. In vitro cleavage assays are then performed according to the methods of Example 4. The assay results are compared with those of Example 4 to assess activity levels of variant RNPs formed with protein incubated at 25° C.

The methods of this Example allow for comparison of the stability of wild-type and variant Cas12i3 polypeptides and wild-type and variant RNPs (binary complexes). An effector polypeptide demonstrating greater specific binding to an RNA guide than another effector polypeptide to the RNA guide is indicative of a more stable polypeptide. An effector RNP demonstrating more robust in vitro cleavage of a target DNA than cleavage by another effector polypeptide is indicative of a more stable binary complex.

### Example 6—DNA Electrophoretic Mobility Shift Assay for Variant Ternary Complex Detection

This Example describes use of a DNA EMSA to determine the ability of an RNA guide, an effector polypeptide (wild-type or variant Cas12i3 polypeptide), and a target DNA substrate to form a ternary complex.

Effector vectors are transformed into E. coli BL21 (DE3) (New England BioLabs) and expressed under a T7 promoter. Transformed cells are initially grown overnight in 5 mL Luria Broth (Teknova)+50 μg/mL kanamycin, followed by inoculation into 1 L Terrific Broth media (Teknova)+50 ng/ml kanamycin. Cells are grown at 37° C. until an OD₆₀₀ of 0.6-0.8, then protein expression is induced with 0.5 mM IPTG. Cultures are then grown at 18° C. for an additional 14-18 hours. Cultures are harvested and pelleted via centrifugation, then resuspended in 1 mL extraction buffer per 5 g cell pellet (50 mM HEPES. pH 7.5, 500 mM NaCl, 5% glycerol, 0.5 mM TCEP). Cells are lysed via cell disruptor (Constant System Limited), then centrifuged at 20,000×g for 20 minutes at 4° C. in order to clarify the lysate. 0.2% polyethylenimine (PEI) is added to the clarified lysate and incubated at 4° C. with constant end-over-end rotation for 20 minutes. The lysate is then centrifuged again at 20.000×g for 10 minutes. The lysate is purified via ion exchange chromatography. After purification, fractions are run on SDS-PAGE gels, and fractions containing protein of the appropriate size are pooled and concentrated using 30 kD Amicon Ultra15 Centrifugal Units. Proteins are buffer exchanged into 12.5 mM HEPES pH 7.0, 120 mM NaCl, 0.5 mM TCEP, and 50% glycerol. Concentrations are then measured using the Nanodrop (Thermofisher) and proteins are stored at −20° C.

RNPs are prepared using a 2:1 ratio of synthetic RNA guide (Integrated DNA Technologies) to polypeptide. Targets adjacent to the PAM sequences disclosed herein are selected, and RNA guides are designed using a direct repeat sequence as described herein. The RNPs are complexed for 30 minutes at 37° C. in 1×NEB2 buffer (50 mM NaCl, 10 mM Tris-HCl, 10 mM MgCl₂, 1 mM DTT, pH 7.9). After complexing, a 5 point 1:2 serial dilution from 5 μM to 37.5 μM is performed, using 1×NEB2 as a dilution buffer. Apo reactions (polypeptide without RNA guide) are prepared in the same manner, making up the volume of RNA guide with H₂O.

dsDNA target substrates are generated by PCR from an oligo (Integrated DNA Technologies). Before PCR, the 5′ end of the forward primer is labeled an IR800 dye, as described in Yan et al., 2018. Using Amplitaq Gold (Thermofisher), the dsDNA substrate is then amplified with the IR800 labeled forward primer and unlabeled reverse primer. The resulting dsDNA is purified with a DNA Clean and Concentrator Kit (Zymo) and quantified by Nanodrop (Thermofisher).

RNP samples and Apo (control) samples are diluted 1:10 into 1× binding buffer (50 mM NaCl, 10 mM Tris-HCl, 1 mM TCEP, 10% glycerol, 2 mM EDTA, pH 8.0) plus 20 nM IR800 labeled target DNA substrate and mixed thoroughly. Reactions are incubated at 37° C. for 1 hour. Bromophenol blue is added to the reactions for dye front visualization, then the entire reaction is loaded onto a 6% DNA Retardation Gel (Thermofisher), which ran for 90 minutes at 80V. The gel is imaged on the Licor Odyssey CLx.

In this assay, the rate at which DNA migrates through the gel is determined by its size. A DNA only sample is able to migrate a particular distance. However, if an RNP binds to the DNA, a band that represents a larger, less mobile DNA complex appears, which is “upshifted” on the gel.

This Example shows how the affinity of variant RNPs (variant binary complexes) to DNA targets (to produce a ternary complex) can be compared to the affinity of wild-type RNPs (wild-type binary complexes to the DNA targets.

### Example 7—Targeting of Mammalian Genes by Variant Effectors

This Example describes indel assessment on multiple targets using wild-type Cas12i3 or Cas12i3 variants introduced into mammalian cells by transient transfection.

Wild-type Cas12i3 (SEQ ID NO: 3) and Cas12i3 variants comprising the substitutions of Table 3 and Table 4 were cloned into a pcda3.1 backbone (Invitrogen). RNA guides were cloned into a pUC19 backbone (New England Biolabs). The plasmids were then maxi-prepped and diluted to 1 μg/μL. The RNA guide and target sequences are shown in Table 5.

TABLE 5
Mammalian targets and corresponding crRNAs.

| Target | | |
|---|---|---|
| identifier | crRNA sequence | Target sequence |
| AAVS1_ | AUAGUGUGUCCUUAGUUGACAUGUG | GTGAGAATGGTGCGTCCTAG |
| T3 | AGAAUGGUGCGUCCUAG (SEQ ID NO: 8) | (SEQ ID NO: 9) |
| EMX1_ | AUAGUGUGUCCUUAGUUGACAUUAU | TATTATTCCCATAGGGAAGG |
| T5 | UAUUCCCAUAGGGAAGG (SEQ ID NO: 10) | (SEQ ID NO: 11) |
| VEGFA_ | AUAGUGUGUCCUUAGUUGACAUAAU | AATCCTCCACCAGTCATGGT |
| T2 | CCUCCACCAGUCAUGGU (SEQ ID NO: 12) | (SEQ ID NO: 13) |

Approximately 16 hours prior to transfection, 100 μl of 25,000 HEK293T cells in DMEM/10% FBS+Pen/Strep were plated into each well of a 96-well plate. On the day of transfection, the cells were 70-90% confluent. For each well to be transfected, a mixture of 0.5 μl of Lipofectamine 2000 and 9.5 μl of Opti-MEM was prepared and then incubated at room temperature for 5-20 minutes (Solution 1). After incubation, the lipofectamine:OptiMEM mixture was added to a separate mixture containing 126 ng of effector plasmid and 174 ng of guide plasmid and water up to 10 μL (Solution 2). In the case of negative controls, the crRNA was not included in Solution 2. The solution 1 and solution 2 mixtures were mixed by pipetting up and down and then incubated at room temperature for 25 minutes. Following incubation, 20 μL of the Solution 1 and Solution 2 mixture were added dropwise to each well of a 96 well plate containing the cells. 72 hours post transfection, cells are trypsinized by adding 10 μL of TrypLE to the center of each well and incubated for approximately 5 minutes. 100 μL of D10 media was then added to each well and mixed to resuspend cells. The cells were then spun down at 500 g for 10 minutes, and the supernatant was discarded. QuickExtract buffer was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit.

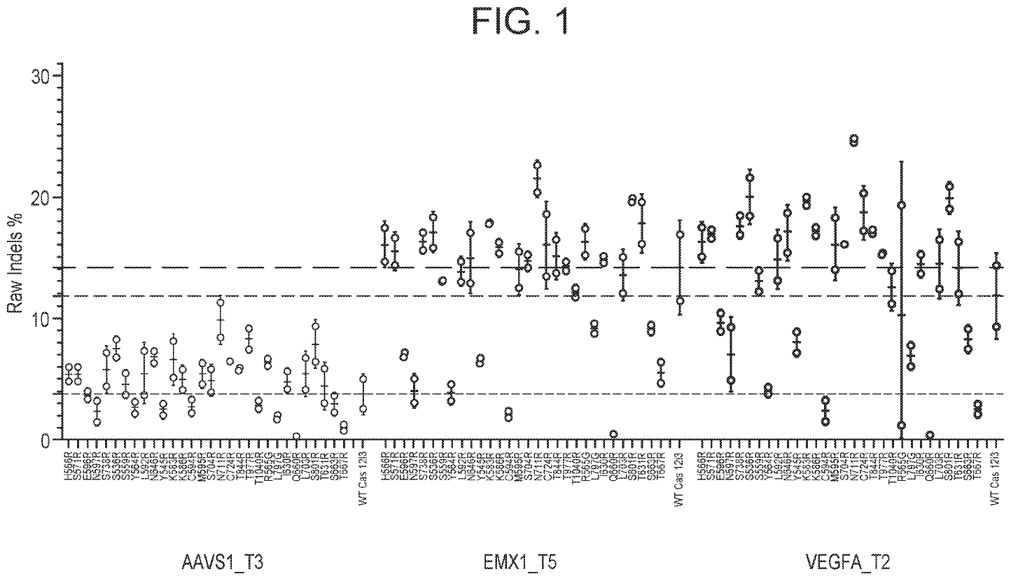

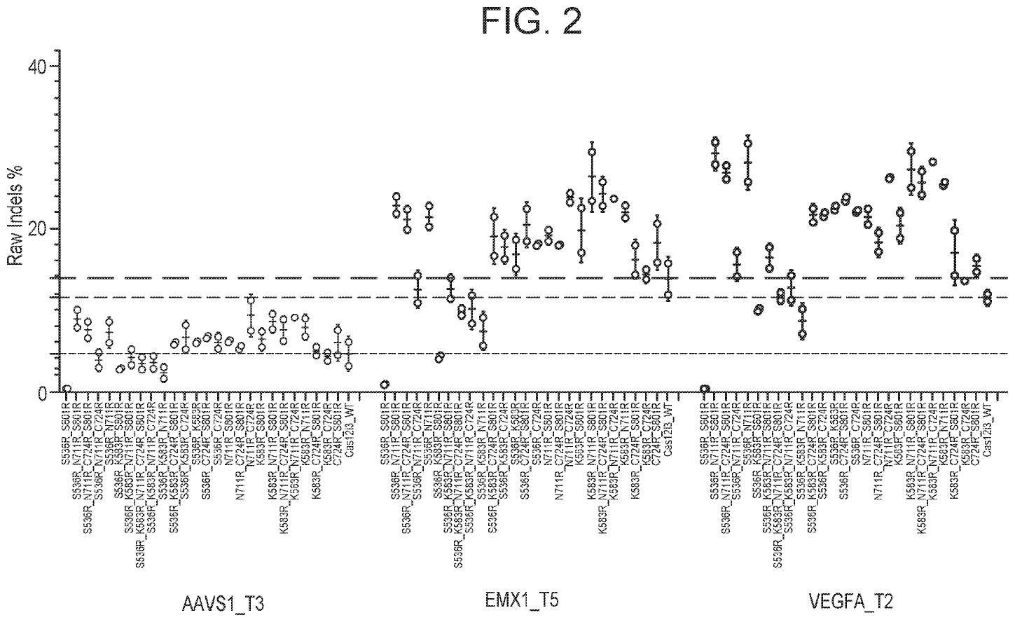

FIG. 1 shows indel activity for wild-type Cas12i3 of SEQ ID NO: 3 and Cas12i3 variants comprising a single substitution relative to SEQ ID NO: 3. FIG. 2 shows indel activity for wild-type Cas12i3 of SEQ ID NO: 3 and Cas12i3 variants comprising multiple substitutions relative to SEQ ID NO: 3. Multiple Cas12i3 variants demonstrated higher % indels relative to wild-type Cas12i3. Fold change in % indels for Cas12i3 variants over % indels for wild-type Cas12i3 at each of the three targets is shown in Table 6.

TABLE 6
Fold change in % indels.

| Substitution(s) | AAVS1_T3 | EMX1_T5 | VEGFA_T2 | Average |
|---|---|---|---|---|
| H566R | 1.53 | 1.16 | 1.42 | 1.37 |
| S571R | 1.54 | 1.12 | 1.5 | 1.39 |
| S738R | 1.58 | 1.18 | 1.55 | 1.44 |
| S536R | 2.16 | 1.27 | 1.8 | 1.74 |
| S559R | 1.28 | 0.95 | 1.17 | 1.13 |
| L592R | 1.45 | 1 | 1.28 | 1.24 |
| N646R | 1.98 | 1.12 | 1.54 | 1.55 |
| K583R | 1.81 | 1.3 | 1.74 | 1.61 |
| K586R | 1.39 | 1.15 | 1.51 | 1.35 |
| M595R | 1.53 | 1.05 | 1.46 | 1.35 |
| S704R | 1.36 | 1.07 | 1.43 | 1.28 |
| N711R | 2.79 | 1.56 | 2.17 | 2.17 |
| C724R | 1.92 | 1.21 | 1.68 | 1.6 |
| T844R | 1.72 | 1.13 | 1.52 | 1.45 |
| T977R | 2.37 | 1.04 | 1.35 | 1.59 |
| R565G | 1.86 | 1.21 | 0.73 | 1.27 |
| I630R | 1.39 | 1.08 | 1.26 | 1.24 |
| L703R | 1.48 | 0.97 | 1.24 | 1.23 |
| S801R | 2.2 | 1.44 | 1.74 | 1.79 |
| T631R | 1.18 | 1.28 | 1.21 | 1.22 |
| S536R N711R S801R | 2.1 | 1.68 | 2.55 | 2.11 |
| S536R N711R C724R S801R | 1.77 | 1.55 | 2.35 | 1.89 |
| S536R N711R C724R | 0.87 | 0.9 | 1.35 | 1.04 |
| S536R N711R | 1.67 | 1.57 | 2.45 | 1.9 |
| S536R K583R N711R S801R | 0.94 | 0.92 | 1.43 | 1.1 |
| S536R K583R N711R C724R | 0.8 | 0.72 | 1.1 | 0.88 |
| S536R K583R C724R S801R | 1.43 | 1.38 | 1.89 | 1.57 |
| S536R K583R C724R | 1.51 | 1.29 | 1.9 | 1.57 |
| S536R K583R | 1.48 | 1.22 | 1.97 | 1.56 |
| S536R C724R S801R | 1.63 | 1.49 | 2.06 | 1.73 |
| S536R C724R | 1.41 | 1.33 | 1.93 | 1.56 |
| N711R S801R | 1.52 | 1.42 | 1.88 | 1.6 |
| N711R C724R S801R | 1.33 | 1.33 | 1.6 | 1.42 |
| N711R C724R | 2.15 | 1.75 | 2.29 | 2.06 |
| K583R S801R | 1.5 | 1.43 | 1.77 | 1.57 |
| K583R N711R S801R | 2.03 | 1.92 | 2.37 | 2.11 |
| K583R N711R C724R S801R | 1.75 | 1.78 | 2.23 | 1.92 |
| K583R N711R C724R | 2.96 | 1.99 | 2.57 | 2.51 |
| K583R N711R | 1.83 | 1.62 | 2.22 | 1.89 |
| K583R C724R S801R | 1.26 | 1.21 | 1.49 | 1.32 |
| K583R C724R | 1 | 1.05 | 1.18 | 1.08 |
| C724R S801R | 1.34 | 1.32 | 1.35 | 1.34 |

Therefore, engineered Cas12i3 variants demonstrated increased nuclease activity in mammalian cells.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the present disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A variant Cas12i3 polypeptide, wherein the variant Cas12i3 polypeptide comprises an amino acid sequence at least 95% identical to SEQ ID NO: 3 and comprises one or more mutations relative to a wild-type Cas12i3 polypeptide of SEQ ID NO: 3.

2. The variant Cas12i3 polypeptide of claim 1, wherein the one or more mutations in the variant Cas12i3 polypeptide are at one or more of positions S536, S559, R565, H566, S571, K583, K586, L592, M595, E596, I630, T631, N646, L703, S704, N711, C724, S738, S801, T844, T977, and/or T1040 of SEQ ID NO: 3.

3. The variant Cas12i3 polypeptide of claim 1 or 2, wherein the one or more mutations are amino acid substitutions, which optionally are one or more of S536R, S559R, R565G, H566R, S571R, K583R, K586R, L592R, M595R, E596R, I630R, T631R, N646R, L703R, S704R, N711R, C724R, S738R, S801R, T844R, T977R, T1040R, or a combination thereof.

4. The variant Cas12i3 polypeptide of any one of claims 1-3, wherein the variant Cas12i3 polypeptide comprises:
a. mutations at positions S536, N711, and S801, which optionally are amino acid substitutions of S536R, N711R, and S801R;
b. mutations at positions S536, N711, C724, and S801, which optionally are amino acid substitutions of S536R, N711R, C724R, and S801R;
c. mutations at positions S536, N711, and C724, which optionally are amino acid substitutions of S536R, N711R, and C724R;
d. mutations at positions S536 and N711, which optionally are amino acid substitutions of S536R and N711R;
e. mutations at positions S536, K583, N711, and S801, which optionally are amino acid substitutions of S536R, K583R, N711R, and S801R;
f. mutations at positions S536, K583, N711, and C724, which optionally are amino acid substitutions of S536R, K583R, N711R, and C724R;
g. mutations at positions S536, K583, C724, and S801, which optionally are amino acid substitutions of S536R, K583R, C724R, and S801R;
h. mutations at positions S536, K583, and C724, which optionally are amino acid substitutions of S536R, K583R, and C724R;
i. mutations at positions S536 and K583, which optionally are amino acid substitutions of S536R and K583R;
j. mutations at positions S536, C724, and S801, which optionally are amino acid substitutions of S536R, C724R, and S801R;
k. mutations at positions S536 and C724, which optionally are amino acid substitutions of S536R and C724R;
l. mutations at positions N711 and S801, which optionally are amino acid substitutions of N711R and S801R;
m. mutations at positions N711, C724, and S801, which optionally are amino acid substitutions of N711R, C724R, and S801R;
n. mutations at positions N711 and C724, which optionally are amino acid substitutions of N711R and C724R;
o. mutations at positions K583 and S801, which optionally are amino acid substitutions of K583R and S801R;
p. mutations at positions K583, N711, and S801, which optionally are amino acid substitutions of K583R, N711R, and S801R;
q. mutations at positions K583, N711, C724, and S801, which optionally are amino acid substitutions of K583R, N711R, C724R, and S801R;
r. mutations at positions K583, N711, and C724, which optionally are amino acid substitutions of K583R, N711R, and C724R;
S. mutations at positions K583 and N711, which optionally are amino acid substitutions of K583R and N711R;
t. mutations at positions K583, C724, and S801, which optionally are amino acid substitutions of K583R, C724R, and S801R;
u. mutations at positions K583 and C724, which optionally are amino acid substitutions of K583R and C724R; or
v. mutations at positions C724 and S801, which optionally are amino acid substitutions of C724R and S801R.

5. The variant Cas12i3 polypeptide of any one of claims 1-4, wherein the variant Cas12i3 polypeptide comprises an amino acid sequence at least 95% identical to SEQ ID NO: 7 and comprises mutations at positions K583, N711, and C724.

6. The variant Cas12i3 polypeptide of claim 5, wherein the mutations are amino acid substitutions of K583R, N711R, and C724R.

7. The variant Cas12i3 polypeptide of any one of claims 1-6, further comprising one or more substitutions of Table 2.

8. The variant Cas12i3 polypeptide of any one of claims 1-7, wherein the variant Cas12i3 polypeptide comprises the amino acid sequence of SEQ ID NO: 7.

9. The variant Cas12i3 polypeptide of any one of claims 1-8, wherein the variant Cas12i3 polypeptide comprises a RuvC domain or a split RuvC domain.

10. The variant Cas12i3 polypeptide of any one of claims 1-9, wherein the variant Cas12i3 polypeptide comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

11. A first nucleic acid encoding a variant Cas12i3 polypeptide of any of claims 1-10.

12. A gene editing system comprising the variant Cas12i3 polypeptide of any one of claims 1-10.

13. A gene editing system comprising the first nucleic acid of claim 11.

14. The gene editing system of claim 13, wherein the first nucleic acid is a messenger RNA (mRNA).

15. The gene editing system of claim 13 or 14, wherein the first nucleic acid is codon-optimized for expression in a cell.

16. The gene editing system of any one of claims 13-15, wherein the first nucleic acid is included in a viral vector, which optionally is an adeno-associated viral (AAV) vector.

17. The gene editing system of any one of claims 12-16, further comprising an RNA guide or a second nucleic acid encoding the RNA guide.

18. The gene editing system of claim 17, wherein the RNA guide comprises a direct repeat sequence and a spacer sequence.

19. The gene editing system of claim 18, wherein the direct repeat comprises:
a. nucleotide 1 through nucleotide 36 of a sequence that is at least 90% identical to SEQ ID NO: 4 or SEQ ID NO: 5;
b. nucleotide 2 through nucleotide 36 of a sequence that is at least 90% identical to SEQ ID NO: 4 or SEQ ID NO: 5;
c. nucleotide 3 through nucleotide 36 of a sequence that is at least 90% identical to SEQ ID NO: 4 or SEQ ID NO: 5;
d. nucleotide 4 through nucleotide 36 of a sequence that is at least 90% identical to SEQ ID NO: 4 or SEQ ID NO: 5;
e. nucleotide 5 through nucleotide 36 of a sequence that is at least 90% identical to SEQ ID NO: 4 or SEQ ID NO: 5;
f. nucleotide 6 through nucleotide 36 of a sequence that is at least 90% identical to SEQ ID NO: 4 or SEQ ID NO: 5;
g. nucleotide 7 through nucleotide 36 of a sequence that is at least 90% identical to SEQ ID NO: 4 or SEQ ID NO: 5;
h. nucleotide 8 through nucleotide 36 of a sequence that is at least 90% identical to SEQ ID NO: 4 or SEQ ID NO: 5;
i. nucleotide 9 through nucleotide 36 of a sequence that is at least 90% identical to SEQ ID NO: 4 or SEQ ID NO: 5;
j. nucleotide 10 through nucleotide 36 of a sequence that is at least 90% identical to SEQ ID NO: 4 or SEQ ID NO: 5;
k. nucleotide 11 through nucleotide 36 of a sequence that is at least 90% identical to SEQ ID NO: 4 or SEQ ID NO: 5;
l. nucleotide 12 through nucleotide 36 of a sequence that is at least 90% identical to SEQ ID NO: 4 or SEQ ID NO: 5;
m. nucleotide 13 through nucleotide 36 of a sequence that is at least 90% identical to SEQ ID NO: 4 or SEQ ID NO: 5;
n. nucleotide 14 through nucleotide 36 of a sequence that is at least 90% identical to SEQ ID NO: 4 or SEQ ID NO: 5; or
o. a sequence that is at least 90% identical to a sequence of SEQ ID NO: 6 or a portion thereof.

20. The gene editing system of claim 18 or 19, wherein the direct repeat comprises:
a. nucleotide 1 through nucleotide 36 of a sequence that is at least 95% identical to SEQ ID NO: 4 or SEQ ID NO: 5;
b. nucleotide 2 through nucleotide 36 of a sequence that is at least 95% identical to SEQ ID NO: 4 or SEQ ID NO: 5;
c. nucleotide 3 through nucleotide 36 of a sequence that is at least 95% identical to SEQ ID NO: 4 or SEQ ID NO: 5;
d. nucleotide 4 through nucleotide 36 of a sequence that is at least 95% identical to SEQ ID NO: 4 or SEQ ID NO: 5;
e. nucleotide 5 through nucleotide 36 of a sequence that is at least 95% identical to SEQ ID NO: 4 or SEQ ID NO: 5;
f. nucleotide 6 through nucleotide 36 of a sequence that is at least 95% identical to SEQ ID NO: 4 or SEQ ID NO: 5;
g. nucleotide 7 through nucleotide 36 of a sequence that is at least 95% identical to SEQ ID NO: 4 or SEQ ID NO: 5;
h. nucleotide 8 through nucleotide 36 of a sequence that is at least 95% identical to SEQ ID NO: 4 or SEQ ID NO: 5;
i. nucleotide 9 through nucleotide 36 of a sequence that is at least 95% identical to SEQ ID NO: 4 or SEQ ID NO: 5;
j. nucleotide 10 through nucleotide 36 of a sequence that is at least 95% identical to SEQ ID NO: 4 or SEQ ID NO: 5;
k. nucleotide 11 through nucleotide 36 of a sequence that is at least 95% identical to SEQ ID NO: 4 or SEQ ID NO: 5;
l. nucleotide 12 through nucleotide 36 of a sequence that is at least 95% identical to SEQ ID NO: 4 or SEQ ID NO: 5;
m. nucleotide 13 through nucleotide 36 of a sequence that is at least 95% identical to SEQ ID NO: 4 or SEQ ID NO: 5;
n. nucleotide 14 through nucleotide 36 of a sequence that is at least 95% identical to SEQ ID NO: 4 or SEQ ID NO: 5; or
o. a sequence that is at least 95% identical to a sequence of SEQ ID NO: 6 or a portion thereof.

21. The gene editing system of any one of claims 18-20, wherein the direct repeat comprises:
a. nucleotide 1 through nucleotide 36 of SEQ ID NO: 4 or SEQ ID NO: 5;
b. nucleotide 2 through nucleotide 36 of SEQ ID NO: 4 or SEQ ID NO: 5;
c. nucleotide 3 through nucleotide 36 of SEQ ID NO: 4 or SEQ ID NO: 5;
d. nucleotide 4 through nucleotide 36 of SEQ ID NO: 4 or SEQ ID NO: 5;
e. nucleotide 5 through nucleotide 36 of SEQ ID NO: 4 or SEQ ID NO: 5;
f. nucleotide 6 through nucleotide 36 of SEQ ID NO: 4 or SEQ ID NO: 5;
g. nucleotide 7 through nucleotide 36 of SEQ ID NO: 4 or SEQ ID NO: 5;
h. nucleotide 8 through nucleotide 36 of SEQ ID NO: 4 or SEQ ID NO: 5;
i. nucleotide 9 through nucleotide 36 of SEQ ID NO: 4 or SEQ ID NO: 5;
j. nucleotide 10 through nucleotide 36 of SEQ ID NO: 4 or SEQ ID NO: 5;
k. nucleotide 11 through nucleotide 36 of SEQ ID NO: 4 or SEQ ID NO: 5;
l. nucleotide 12 through nucleotide 36 of SEQ ID NO: 4 or SEQ ID NO: 5;
m. nucleotide 13 through nucleotide 36 of SEQ ID NO: 4 or SEQ ID NO: 5;
n. nucleotide 14 through nucleotide 36 of SEQ ID NO: 4 or SEQ ID NO: 5; or
o. SEQ ID NO: 6 or a portion thereof.

22. The gene editing system of any one of claims 18-21, wherein the spacer sequence comprises between 15 and 35 nucleotides in length.

23. The gene editing system of any one of claims 17-22, wherein the system comprises the second nucleic acid encoding the RNA guide.

24. The gene editing system of claim 23, wherein the second nucleic acid encoding the RNA guide is located in a viral vector.

25. The gene editing system of claim 24, wherein the viral vector comprises the both the first nucleic acid encoding the Cas12i3 polypeptide and the second nucleic acid encoding the RNA guide.

26. The gene editing system of any one of claims 23-25, wherein the system comprises the first nucleic acid encoding the Cas12i3 polypeptide, which is located in a first viral vector, and wherein the system comprises the second nucleic acid encoding the RNA guide, which is located in a second viral vector.

27. The gene editing system of any one of claims 12-26, wherein the gene editing system is present in a delivery composition comprising a nanoparticle, a liposome, an exosome, a microvesicle, or a gene-gun.

28. The gene editing system of any one of claims 18-27, wherein the spacer sequence is specific to a target sequence within a target nucleic acid.

29. The gene editing system of claim 28, wherein the target sequence is adjacent to a protospacer adjacent motif (PAM) comprising the motif of 5′-TTN-3′ or 5′-NTTN-3′.

30. The variant polypeptide or the gene editing system of any previous claim, wherein the variant Cas12i3 polypeptide exhibits enhanced enzymatic activity (e.g., nuclease activity), enhanced binding activity (e.g., binding activity to an RNA guide), enhanced binding specificity (e.g., binding specificity to an RNA guide), and/or enhanced stability relative to the wild-type Cas12i3 polypeptide of SEQ ID NO: 3.

31. The gene editing system of any previous claim, wherein the variant Cas12i3 polypeptide and the RNA guide exhibit enhanced binding activity to a target nucleic acid, enhanced binding specificity to the target nucleic acid, and/or enhanced stability relative to the wild-type Cas12i3 polypeptide of SEQ ID NO: 3 and the RNA guide.

32. A composition comprising the variant Cas12i3 polypeptide or the gene editing system of any previous claim.

33. The composition of claim 32, wherein the composition is a pharmaceutical composition.

34. A cell comprising the variant Cas12i3 polypeptide, the gene editing system, or the composition of any previous claim.

35. The cell of claim 34, wherein the cell is a eukaryotic cell or a prokaryotic cell.

36. The cell of claim 34 or 35, wherein the cell is a mammalian cell or a plant cell.

37. The cell of any one of claims 34-36, wherein the cell is a human cell.

38. A method of producing the variant Cas12i3 polypeptide, the gene editing system, or the composition of any previous claim.

39. The method of claim 38, wherein the method is for producing the variant Cas12i3 polypeptide and the method comprises transforming a host cell with an expression vector expressing a first nucleic acid of claim 11.

40. The method of claim 39, wherein the host cell is ex vivo or in vitro.

41. The method of claim 39, wherein the host cell is in vivo.

42. A method of delivering the variant Cas12i3 polypeptide, the gene editing system, or the composition of any previous claim.

43. The method of claim 42, wherein the Cas12i3 polypeptide, the gene editing system, or the composition is present within a delivery composition, which optionally is selected from the group consisting of a nanoparticle, a liposome, an exosome, a microvesicle, or a gene-gun.

44. The method of claim 43, wherein the delivery composition is delivered to a cell using a method selected from the group consisting of transfection, electroporation, nucleofection, viral delivery, microinjection, microprojectile bombardment, fugene, direct sonic loading, cell squeezing, optical transfection, protoplast fusion, impalefection, magnetofection, exosome-mediated transfer, lipid nanoparticle-mediated transfer, and any combination thereof.

45. The method of any one of claims 42-44, wherein the delivery is to a cell, which is a eukaryotic cell or a prokaryotic cell.

46. The method of claim 45, wherein the cell is a mammalian cell or a plant cell.

47. The method of claim 45 or 46, wherein the cell is a human cell.

48. A method of editing a cell, comprising administering the gene editing system or composition of any previous claim to a cell.

49. The method of claim 48, wherein the gene editing system is comprised within a delivery composition, which optionally is selected from the group consisting of a nanoparticle, a liposome, an exosome, a microvesicle, or a gene-gun.

50. The method of claim 48 or 49, wherein the cell is ex vivo or in vitro.

51. The method of claim 48 or 49, wherein the cell is in vivo.

52. The method of any one of claims 48-51, wherein the cell is a eukaryotic cell or a prokaryotic cell.

53. The method of any one of claims 48-52, wherein the cell is a mammalian cell or a plant cell.

54. The method of any one of claims 48-53, wherein the cell is a human cell.


SEQUENCE LISTING (13 sequences)

SEQ ID NO: 1 - DNA, 3294 bp, Unidentified
  source 1..3294: mol_type = unassigned DNA
atgtccatctctaacaataacattctgccgtacaacccgaagctgttgccggacgatcgtaaacacaagatgctggtggataccttcaaccagctggacttgattcgcaataacctgcatgatatgatcattgcgttgtacggtgcgctgaagtatgacaacatcaaacaattcgcgagcaaagagaagccgcacattagcgctgatgcactgtgcagcatcaactggtttcgtctggttaagaccaacgagcgcaaaccggcgattgaatccaaccagatcatttctaagtttatccaatacagcggtcacacgccggataaatatgcgctgagccatattaccggcaaccacgagccgagccataagtggatcgactgtcgtgaatacgcgatcaactatgcccgcattatgcacctgagcttctcccagtttcaagacttggccaccgcgtgcctgaattgtaaaatcctgattttgaacggcaccctgaccagcagctgggcttggggtgcgaacagcgcactgttcggtggtagcgataaggagaactttagcgtgaaagcgaagatcctgaacagcttcattgagaacctgaaagacgaaatgaacaccacgaagtttcaggtggttgaaaaagtttgccagcaaatcggtagcagcgacgcggctgatctgttcgacttgtaccgtagcaccgttaaagatggtaaccgtggtccggcaaccggtcgtaatccgaaagttatgaacctgtttagccaggatggcgaaatcagcagcgagcaacgtgaagacttcatcgagagctttcagaaagtgatgcaagagaagaacagcaagcagatcattccgcacctggataagctgaagtatcatttggttaagcaatccggcctgtacgacatctatagctgggcagccgcgattaaaaatgcgaacagcaccatcgtggctagcaacagcagcaacttgaacaccatcctgaacaaaacggagaagcagcaaaccttcgaggaactgcgtaaagacgaaaagatcgtcgcgtgctctaagattctgttgtccgttaatgataccttgccggaggacctgcactacaacccgtctaccagcaatttgggtaaaaacctggatgtgttctttgacctgttgaatgagaacagcgttcataccattgaaaacaaagaggagaagaacaagatcgttaaagaatgcgtcaatcagtacatggaggaatgtaaaggcctgaacaagccgccaatgccggtgctgttgaccttcattagcgattatgcacacaagcatcaggcccaagacttcctgagcgctgcaaagatgaacttcatcgacctgaaaatcaagagcattaaagtggtgccgacggttcacggtagcagcccgtacacctggattagcaatctgagcaagaagaacaaggatggtaaaatgattcgtaccccgaacagcagcctgatcggctggatcattccgccggaggaaatccatgaccagaagttcgcaggccaaaacccgatcatttgggccgtgctgcgtgtttattgcaataacaaatgggagatgcaccatttcccgttttccgacagccgtttcttcaccgaagtgtacgcgtataagccgaatctgccgtacttgccgggtggcgagaaccgtagcaaacgctttggttatcgtcacagcaccaatctgtctaacgaaagccgccagatcctgctggacaaaagcaagtacgctaaagcaaacaagagcgttctgcgttgcatggagaatatgacccataacgttgtcttcgatccgaagaccagcctgaacatccgtattaaaaccgacaagaataacagcccggtgctggacgataaaggtcgtatcacctttgttatgcaaatcaaccaccgcattctggagaagtacaacaacaccaagatcgaaatcggcgatcgtattctggcgtatgaccagaatcaaagcgagaaccatacgtatgcgatcctgcagcgtaccgaggaaggtagccacgctcatcaattcaacggctggtacgtgcgcgtgctggaaaccggtaaagtgacgtccattgttcagggtctgagcggcccgatcgatcaactgaattatgacggcatgccggtgaccagccacaagtttaactgctggcaggcggatcgtagcgctttcgtttcccaatttgcgagcctgaaaattagcgaaaccgaaaccttcgacgaggcgtaccaggcgatcaacgcacaaggtgcctacacctggaacctgttttatttgcgtattctgcgcaaggcgctgcgtgtttgccatatggaaaatatcaaccagttccgtgaggaaatcctggcgattagcaaaaaccgcctgtccccgatgtctttgggtagcctgtcccaaaacagcctgaagatgatccgtgcgttcaaaagcatcattaactgttatatgagccgcatgtcctttgtggatgagctgcagaagaaagagggtgacctggagctgcacacgattatgcgtttgaccgataacaagctgaacgacaagcgtgttgagaaaatcaatcgcgcgagcagcttcctgaccaacaaggcacatagcatgggttgcaaaatgattgtgggcgaaagcgatctgccggttgcggactctaagaccagcaagaaacagaacgtggatcgtatggactggtgcgcgcgtgcgctgagccacaaggttgagtacgcgtgtaagctgatgggtctggcataccgtggcatcccggcctatatgagcagccaccaggacccgttggtccatctggtggaatccaaacgttctgtgctgcgtccgcgcttcgtggttgcggataagagcgacgttaaacaacaccacctggacaatctgcgtcgcatgctgaacagcaaaaccaaggtcggcaccgcggtgtactatcgtgaggctgtggagctgatgtgcgaggaactgggtatccacaagaccgatatggcgaagggtaaagtgagcctgagcgatttcgtcgacaagtttatcggcgaaaaagcgattttcccgcagcgtggtggccgcttttacatgagcaccaagcgtttgaccacgggtgcaaaactgatttgttattccggtagcgacgtttggctgagcgacgctgatgagatcgccgcgatcaacattggcatgttcgtcgtgtgcgatcaaaccggcgcgttcaagaaaaagaaaaaggaaaaactggacgatgaggaatgtgacatcctgccgttccgtccgatg

SEQ ID NO: 2 - DNA, 3294 bp, Unidentified
  source 1..3294: mol_type = unassigned DNA
atgtccatctctaacaataacattctgccctacaaccctaagctgctcccagacgatcgaaaacacaagatgctggtggatactttcaatcagctggacctgattcgtaataacctgcatgatatgatcatcgccctgtacggcgctctgaagtatgacaacatcaaacagttcgcctctaaagagaagccccacatttctgccgatgctctgtgcagtatcaattggtttcgactggtgaagaccaacgagcgtaaacctgcaatcgaatccaaccagatcatctctaagtttatccagtacagcggacacactcccgataaatatgctctgagccatattaccggtaaccacgagccttcccataagtggatcgactgtcgggaatacgcaatcaattatgccagaattatgcacctgagtttctcacagtttcaggacctggcaacagcctgcctgaattgtaagatcctgatcctgaacggaacactgactagctcctgggcatggggtgccaattcagctctgttcggcgggagcgataaggagaacttttccgtgaaagccaagatcctgaacagcttcattgagaacctgaaagacgaaatgaacaccacaaagtttcaggtggtcgaaaaagtctgccagcagatcggctctagtgacgccgctgatctgttcgacctgtacaggagcacagtgaaggacggaaatcgaggtccggccactggccgtaatcccaaagtcatgaacctgttttcccaggatggggagatttcaagcgagcagcgcgaagacttcatcgagtcatttcagaaagtgatgcaggagaagaacagcaagcagatcattccccacctggataagctgaagtatcacctggtcaagcagtcagggctgtacgacatctatagctgggcagccgccatcaagaatgcaaacagtaccatcgtggcctcaaattcctctaacctgaacaccatcctgaacaaaacagagaagcagcagactttcgaggaactgcggaaagacgaaaagatcgttgcctgtagcaagattctgctctccgtgaatgataccctccctgaggacctgcactacaacccaagcacatccaacctgggaaagaacctggatgtcttctttgacctgctgaacgagaactccgttcatacaattgaaaataaggaggagaagaacaagatcgtcaaagagtgcgtgaatcagtacatggaggaatgtaaaggcctgaacaagccccctatgcctgtgctgctgaccttcatcagcgattatgcccacaagcatcaggctcaggacttcctgagcgcagccaaaatgaactttatcgacctgaaaatcaagtccattaaggttgtgccgacagtgcacgggagttcaccctacacttggattagcaatctgtccaagaaaaacaaagatggcaagatgattagaacaccaaacagctccctgatcgggtggatcattccaccggaggaaatccatgaccagaagttcgcaggacagaatcctatcatttgggccgtgctgagggtctattgcaataacaaatgggagatgcaccatttcccattttctgatagtcgcttctttaccgaagtgtacgcctataagccaaatctgccgtacctgcccggaggcgagaaccgatccaagcggttcggctataggcactctacaaatctgtctaacgaaagtcgccagatcctgctcgacaagagtaagtacgctaaagcaaacaagtcagtgctgcgatgtatggagaatatgacccataacgtggtgttcgatccaaagaccagtctgaatatccggatcaagacagacaagaataactctcccgtgctggacgataaaggacgaatcacttttgtcatgcagatcaaccaccgtattctggagaagtacaacaacaccaagatcgaaatcggcgataggattctggcctacgaccagaatcagtctgagaaccatacttatgccatcctgcagcggaccgaggaaggaagccacgctcatcagttcaacggttggtacgtgcgggtgctggagaccggaaaggtgacctctattgtccagggcctcagtgggccgatcgatcagctgaattatgacggtatgccagtgacctcccacaagtttaactgctggcaggctgatcggtctgcattcgtcagtcagtttgcctcactgaaaattagcgagaccgaaacattcgacgaggcataccaggccatcaatgctcagggcgcatacacatggaacctgttctacctgcggattctgagaaaggctctgagagtgtgccacatggagaatatcaaccagttccgggaggaaatcctggcaattagcaagaatagactgtcacccatgagcctcgggtcactgagccagaactctctgaagatgatcagggccttcaaaagtatcattaattgttacatgtcccgcatgtcttttgtggatgagctgcagaagaaagagggagacctggaactccacaccattatgcggctgacagataacaagctgaacgacaagcgggtggagaaaatcaatagagcttctagttttctgacaaacaaggcacattccatgggctgcaaaatgatcgtgggggaatctgatctgccagtcgccgacagtaagacttcaaagaaacagaacgtcgataggatggactggtgcgcccgcgctctgagccacaaggttgagtacgcttgtaaactcatgggactggcttaccggggtatccctgcatatatgtcaagccaccaggatccactcgttcatctggtggaaagcaagcggagcgtgctgaggccacgtttcgtggtcgcagataagagcgacgtgaaacagcaccacctggacaatctgcggagaatgctgaactccaaaacaaaggttggcactgccgtgtactatagggaggctgtggaactcatgtgcgaggaactgggcatccacaagactgatatggccaaagggaaggtctccctgtctgatttcgttgacaagtttatcggggagaaagctattttccctcagaggggcgggcgcttttacatgtctaccaagcggctgactaccggcgcaaaactgatttgttatagtgggtcagacgtgtggctgagcgacgccgatgagatcgctgcaatcaacattggaatgttcgtggtgtgcgatcagaccggtgcctttaagaaaaagaaaaaggaaaagctcgacgatgaggaatgtgacatcctgcctttccggccaatg

SEQ ID NO: 3 - protein, 1098 aa, Unidentified
MSISNNNILPYNPKLLPDDRKHKMLVDTFNQLDLIRNNLHDMIIALYGALKYDNIKQFASKEKPHISADALCSINWFRLVKTNERKPAIESNQIISKFIQYSGHTPDKYALSHITGNHEPSHKWIDCREYAINYARIMHLSFSQFQDLATACLNCKILILNGTLTSSWAWGANSALFGGSDKENFSVKAKILNSFIENLKDEMNTTKFQVVEKVCQQIGSSDAADLFDLYRSTVKDGNRGPATGRNPKVMNLFSQDGEISSEQREDFIESFQKVMQEKNSKQIIPHLDKLKYHLVKQSGLYDIYSWAAAIKNANSTIVASNSSNLNTILNKTEKQQTFEELRKDEKIVACSKILLSVNDTLPEDLHYNPSTSNLGKNLDVFFDLLNENSVHTIENKEEKNKIVKECVNQYMEECKGLNKPPMPVLLTFISDYAHKHQAQDFLSAAKMNFIDLKIKSIKVVPTVHGSSPYTWISNLSKKNKDGKMIRTPNSSLIGWIIPPEEIHDQKFAGQNPIIWAVLRVYCNNKWEMHHFPFSDSRFFTEVYAYKPNLPYLPGGENRSKRFGYRHSTNLSNESRQILLDKSKYAKANKSVLRCMENMTHNVVFDPKTSLNIRIKTDKNNSPVLDDKGRITFVMQINHRILEKYNNTKIEIGDRILAYDQNQSENHTYAILQRTEEGSHAHQFNGWYVRVLETGKVTSIVQGLSGPIDQLNYDGMPVTSHKFNCWQADRSAFVSQFASLKISETETFDEAYQAINAQGAYTWNLFYLRILRKALRVCHMENINQFREEILAISKNRLSPMSLGSLSQNSLKMIRAFKSIINCYMSRMSFVDELQKKEGDLELHTIMRLTDNKLNDKRVEKINRASSFLTNKAHSMGCKMIVGESDLPVADSKTSKKQNVDRMDWCARALSHKVEYACKLMGLAYRGIPAYMSSHQDPLVHLVESKRSVLRPRFVVADKSDVKQHHLDNLRRMLNSKTKVGTAVYYREAVELMCEELGIHKTDMAKGKVSLSDFVDKFIGEKAIFPQRGGRFYMSTKRLTTGAKLICYSGSDVWLSDADEIAAINIGMFVVCDQTGAFKKKKKEKLDDEECDILPFRPM

SEQ ID NO: 4 - RNA, 36 nt, synthetic construct
  source 1..36: mol_type = other RNA
ctagcaatgacctaatagtgtgtccttagttgacat

SEQ ID NO: 5 - RNA, 36 nt, synthetic construct
  source 1..36: mol_type = other RNA
cctacaatacctaagaaatccgtcctaagttgacgg

SEQ ID NO: 6 - RNA, 22 nt, synthetic construct
  source 1..22: mol_type = other RNA
atagtgtgtccttagttgacat

SEQ ID NO: 7 - protein, 1098 aa, synthetic construct
MSISNNNILPYNPKLLPDDRKHKMLVDTFNQLDLIRNNLHDMIIALYGALKYDNIKQFASKEKPHISADALCSINWFRLVKTNERKPAIESNQIISKFIQYSGHTPDKYALSHITGNHEPSHKWIDCREYAINYARIMHLSFSQFQDLATACLNCKILILNGTLTSSWAWGANSALFGGSDKENFSVKAKILNSFIENLKDEMNTTKFQVVEKVCQQIGSSDAADLFDLYRSTVKDGNRGPATGRNPKVMNLFSQDGEISSEQREDFIESFQKVMQEKNSKQIIPHLDKLKYHLVKQSGLYDIYSWAAAIKNANSTIVASNSSNLNTILNKTEKQQTFEELRKDEKIVACSKILLSVNDTLPEDLHYNPSTSNLGKNLDVFFDLLNENSVHTIENKEEKNKIVKECVNQYMEECKGLNKPPMPVLLTFISDYAHKHQAQDFLSAAKMNFIDLKIKSIKVVPTVHGSSPYTWISNLSKKNKDGKMIRTPNSSLIGWIIPPEEIHDQKFAGQNPIIWAVLRVYCNNKWEMHHFPFSDSRFFTEVYAYKPNLPYLPGGENRSKRFGYRHSTNLSNESRQILLDKSRYAKANKSVLRCMENMTHNVVFDPKTSLNIRIKTDKNNSPVLDDKGRITFVMQINHRILEKYNNTKIEIGDRILAYDQNQSENHTYAILQRTEEGSHAHQFNGWYVRVLETGKVTSIVQGLSGPIDQLRYDGMPVTSHKFNRWQADRSAFVSQFASLKISETETFDEAYQAINAQGAYTWNLFYLRILRKALRVCHMENINQFREEILAISKNRLSPMSLGSLSQNSLKMIRAFKSIINCYMSRMSFVDELQKKEGDLELHTIMRLTDNKLNDKRVEKINRASSFLTNKAHSMGCKMIVGESDLPVADSKTSKKQNVDRMDWCARALSHKVEYACKLMGLAYRGIPAYMSSHQDPLVHLVESKRSVLRPRFVVADKSDVKQHHLDNLRRMLNSKTKVGTAVYYREAVELMCEELGIHKTDMAKGKVSLSDFVDKFIGEKAIFPQRGGRFYMSTKRLTTGAKLICYSGSDVWLSDADEIAAINIGMFVVCDQTGAFKKKKKEKLDDEECDILPFRPM

SEQ ID NO: 8 - RNA, 42 nt, synthetic construct
  source 1..42: mol_type = other RNA
atagtgtgtccttagttgacatgtgagaatggtgcgtcctag

SEQ ID NO: 9 - DNA, 20 bp, Homo sapiens
  source 1..20: mol_type = unassigned DNA
gtgagaatggtgcgtcctag

SEQ ID NO: 10 - RNA, 42 nt, synthetic construct
  source 1..42: mol_type = other RNA
atagtgtgtccttagttgacattattattcccatagggaagg

SEQ ID NO: 11 - DNA, 20 bp, Homo sapiens
  source 1..20: mol_type = unassigned DNA
tattattcccatagggaagg

SEQ ID NO: 12 - RNA, 42 nt, synthetic construct
  source 1..42: mol_type = other RNA
atagtgtgtccttagttgacataatcctccaccagtcatggt

SEQ ID NO: 13 - DNA, 20 bp, Homo sapiens
  source 1..20: mol_type = unassigned DNA
aatcctccaccagtcatggt